SEQUENCE LISTING (72 sequences)

SEQ ID NO: 1 - protein, 7 aa, Artificial Sequence
  Synthetic peptide
SSNYYWG

SEQ ID NO: 2 - protein, 15 aa, Artificial Sequence
  Synthetic peptide
GSIYYSGNTYYNPSL

SEQ ID NO: 3 - protein, 13 aa, Artificial Sequence
  Synthetic peptide
REWSPYESEGFDY

SEQ ID NO: 4 - protein, 14 aa, Artificial Sequence
  Synthetic peptide
TGSSSNIGAGYDVH

SEQ ID NO: 5 - protein, 7 aa, Artificial Sequence
  Synthetic peptide
GNNNRPS

SEQ ID NO: 6 - protein, 10 aa, Artificial Sequence
  Synthetic peptide
QSYDTGLSAW

SEQ ID NO: 7 - protein, 122 aa, Artificial Sequence
  Synthetic peptide
QLQLQESGPGLVKPSETLSLTCTVSGGSITSSNYYWGWIRQPPGKGLEWIGSIYYSGNTYYNPSLKSRVTISVDTSKNQFSLKLSSVTAADTAVYYCAREWSPYESEGFDYWGQGTLVTVSS

SEQ ID NO: 8 - protein, 111 aa, Artificial Sequence
  Synthetic peptide
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDTGLSAWVFGGGTKLTVL

SEQ ID NO: 9 - protein, 4 aa, Artificial Sequence
  Synthetic peptide
GGGS

SEQ ID NO: 10 - protein, 16 aa, Artificial Sequence
  Synthetic peptide
GGGSGGGSGGGSGGGS

SEQ ID NO: 11 - protein, 5 aa, Artificial Sequence
  Synthetic peptide
GGGGS

SEQ ID NO: 12 - protein, 10 aa, Artificial Sequence
  Synthetic peptide
GGGGSGGGGS

SEQ ID NO: 13 - protein, 15 aa, Artificial Sequence
  Synthetic peptide
GGGGSGGGGSGGGGS

SEQ ID NO: 14 - protein, 20 aa, Artificial Sequence
  Synthetic peptide
GGGGSGGGGSGGGGSGGGGS

SEQ ID NO: 15 - protein, 248 aa, Artificial Sequence
  Synthetic peptide
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDTGLSAWVFGGGTKLTVLGGGGSGGGGSGGGGSQLQLQESGPGLVKPSETLSLTCTVSGGSITSSNYYWGWIRQPPGKGLEWIGSIYYSGNTYYNPSLKSRVTISVDTSKNQFSLKLSSVTAADTAVYYCAREWSPYESEGFDYWGQGTLVTVSS

SEQ ID NO: 16 - DNA, 366 bp, Artificial Sequence
  Synthetic polynucleotide
cagctgcagctacaggagagcggccccggcctggtgaaaccttctgagaccctgagcctgacgtgcaccgtgagcggcggcagcatcaccagcagcaactactactggggctggatacgacagccccctggaaagggcctagagtggatcggcagcatctactacagcggcaacacctactacaaccccagcctgaagagcagagtcaccatcagcgtggacaccagcaagaaccagttcagcctgaagctgagcagcgtgaccgccgccgacacagctgtttactattgtgccagagagtggagcccctacgagagcgagggcttcgactactggggccaggggaccctggtgaccgtttcttct

SEQ ID NO: 17 - DNA, 333 bp, Artificial Sequence
  Synthetic polynucleotide
cagagcgtgctgacacagcctccatctgtttctggcgcccctggccagagagtgaccatctcttgtactggcagcagctccaacattggagccggctatgacgtgcactggtaccagcagctgcctggcacagcccctaaactgctgatctacggcaacaacaacagacccagcggcgtgcccgacagattttctggctccaagagcggcacaagcgccagcctggctattactggactgcaggccgaggacgaggccgactactactgtcagagctacgacaccggcctgagcgcctgggtttttggtggaggcacaaaactgacagtgctg

SEQ ID NO: 18 - DNA, 45 bp, Artificial Sequence
  Synthetic oligonucleotide
ggaggcggaggttctggcggcggaggaagtggtggcggaggctca

SEQ ID NO: 19 - DNA, 744 bp, Artificial Sequence
  Synthetic polynucleotide
cagagcgtgctgacacagcctccatctgtttctggcgcccctggccagagagtgaccatctcttgtactggcagcagctccaacattggagccggctatgacgtgcactggtaccagcagctgcctggcacagcccctaaactgctgatctacggcaacaacaacagacccagcggcgtgcccgacagattttctggctccaagagcggcacaagcgccagcctggctattactggactgcaggccgaggacgaggccgactactactgtcagagctacgacaccggcctgagcgcctgggtttttggtggaggcacaaaactgacagtgctgggaggcggaggttctggcggcggaggaagtggtggcggaggctcacagctgcagctacaggagagcggccccggcctggtgaaaccttctgagaccctgagcctgacgtgcaccgtgagcggcggcagcatcaccagcagcaactactactggggctggatacgacagccccctggaaagggcctagagtggatcggcagcatctactacagcggcaacacctactacaaccccagcctgaagagcagagtcaccatcagcgtggacaccagcaagaaccagttcagcctgaagctgagcagcgtgaccgccgccgacacagctgtttactattgtgccagagagtggagcccctacgagagcgagggcttcgactactggggccaggggaccctggtgaccgtttcttct

SEQ ID NO: 20 - protein, 45 aa, Homo sapiens
TTTPAPRPPTPAPTIASQPLSLRPEACRPAAGGAVHTRGLDFACD

SEQ ID NO: 21 - DNA, 135 bp, Homo sapiens
acaacaacacctgctcctcggcctcctacaccagctcctacaattgccagccagccactgtctctgaggcccgaagcttgcaggcctgctgctggcggagccgtgcatacaagaggactggatttcgcctgcgac

SEQ ID NO: 22 - protein, 24 aa, Homo sapiens
IYIWAPLAGTCGVLLLSLVITLYC

SEQ ID NO: 23 - DNA, 72 bp, Homo sapiens
atctacatctgggcacctctggctggaacctgtggcgtgctgctgctgagcctggtcatcaccctgtattgc

SEQ ID NO: 24 - protein, 27 aa, Homo sapiens
FWVLVVVGGVLACYSLLVTVAFIIFWV

SEQ ID NO: 25 - DNA, 81 bp, Homo sapiens
ttttgggtgctggtggtggttggtggagtcctggcttgctatagcttgctagtaacagtggcctttattattttctgggtg

SEQ ID NO: 26 - protein, 27 aa, Homo sapiens
IISFFLALTSTALLFLLFFLTLRFSVV

SEQ ID NO: 27 - DNA, 81 bp, Homo sapiens
atcatctccttctttcttgcgctgacctcgactgcgttgctcttcctgctgttcttcctcacgctccgtttctctgttgtt

SEQ ID NO: 28 - protein, 30 aa, Homo sapiens
FALPVGLIVGVTALGLLIIGVVNCVIMTQV

SEQ ID NO: 29 - DNA, 90 bp, Homo sapiens
ttcgctcttccagttggactgattgtgggtgtgacagccttgggtctactaataataggagtggtgaactgtgtcatcatgacccaggtg

SEQ ID NO: 30 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 336 bp, Homo sapiens
agagtgaagttcagcagatccgccgacgctcctgcctatcagcagggccaaaaccagctctacaacgagctgaacctggggagaagagaagagtacgacgtgctggacaagcggagaggcagagatcctgaaatgggcggcaagcccagacggaagaatcctcaagagggcctgtataatgagctacagaaagacaagatggcagaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 32 - protein, 41 aa, Homo sapiens
RSKRSRLLHSDYMNMTPRRPGPTRKHYQPYAPPRDFAAYRS

SEQ ID NO: 33 - DNA, 123 bp, Homo sapiens
cggagcaagagaagcagactgctgcacagcgactacatgaacatgacccctagacggcccggacctaccagaaagcactaccagccttacgctcctcctagagacttcgccgcctacagatcc

SEQ ID NO: 34 - protein, 42 aa, Homo sapiens
KRGRKKLLYIFKQPFMRPVQTTQEEDGCSCRFPEEEEGGCEL

SEQ ID NO: 35 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 36 - protein, 48 aa, Homo sapiens
QRRKYRSNKGESPVEPAEPCRYSCPREEEGSTIPIQEDYRKPEPACSP

SEQ ID NO: 37 - DNA, 144 bp, Homo sapiens
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 38 - protein, 174 aa, Homo sapiens
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 39 - DNA, 522 bp, Homo sapiens
aaaaagaagcccttgtgcctgcagagagaagccaaggtgcctcacttgcctgcagataaggccaggggtacacagggtcccgagcagcagcacctcctgatcacagcgccgagctccagcagcagctccctggagagctcggccagtgcgctagacagaagggcccccactcggaaccagccacaggcaccaggcgtggaggcgagtggggctggggaggcgagggcaagcaccgggagctcagattcttcccctggtggtcatggcacccaggtcaatgtcacctgtatcgtgaacgtctgtagcagctctgaccacagctcacagtgctcctcccaagcctcgtccacaatgggagacacagattccagcccctcggagtccccgaaggacgagcaagtacccttctccaaggaggaatgtgcctttcggtcacagctggagacgccagagaccctgctggggagcaccgaagagaagcccctgccccttggagtgcctgatgctgggatgaagcccagt

SEQ ID NO: 40 - protein, 21 aa, Homo sapiens
MALPVTALLLPLALLLHAARP

SEQ ID NO: 41 - DNA, 63 bp, Homo sapiens
atggctctgcctgtgacagctctgctgctgcctctggctctgcttcttcatgccgccagacca

SEQ ID NO: 42 - protein, 11 aa, Artificial Sequence
  Synthetic peptide
SYPYDVPDYAS

SEQ ID NO: 43 - DNA, 33 bp, Artificial Sequence
  Synthetic oligonucleotide
tcttacccctacgatgtgcctgattatgcatct

SEQ ID NO: 44 - protein, 470 aa, Artificial Sequence
  Synthetic peptide
QSVLTQPPSVSGAPGQRVTISCTGSSSNIGAGYDVHWYQQLPGTAPKLLIYGNNNRPSGVPDRFSGSKSGTSASLAITGLQAEDEADYYCQSYDTGLSAWVFGGGTKLTVLGGGGSGGGGSGGGGSQLQLQESGPGLVKPSETLSLTCTVSGGSITSSNYYWGWIRQPPGKGLEWIGSIYYSGNTYYNPSLKSRVTISVDTSKNQFSLKLSSVTAADTAVYYCAREWSPYESEGFDYWGQGTLVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 1410 bp, Artificial Sequence
  Synthetic polynucleotide
cagagcgtgctgacacagcctccatctgtttctggcgcccctggccagagagtgaccatctcttgtactggcagcagctccaacattggagccggctatgacgtgcactggtaccagcagctgcctggcacagcccctaaactgctgatctacggcaacaacaacagacccagcggcgtgcccgacagattttctggctccaagagcggcacaagcgccagcctggctattactggactgcaggccgaggacgaggccgactactactgtcagagctacgacaccggcctgagcgcctgggtttttggtggaggcacaaaactgacagtgctgggaggcggaggttctggcggcggaggaagtggtggcggaggctcacagctgcagctacaggagagcggccccggcctggtgaaaccttctgagaccctgagcctgacgtgcaccgtgagcggcggcagcatcaccagcagcaactactactggggctggatacgacagccccctggaaagggcctagagtggatcggcagcatctactacagcggcaacacctactacaaccccagcctgaagagcagagtcaccatcagcgtggacaccagcaagaaccagttcagcctgaagctgagcagcgtgaccgccgccgacacagctgtttactattgtgccagagagtggagcccctacgagagcgagggcttcgactactggggccaggggaccctggtgaccgtttcttctacaacaacacctgctcctcggcctcctacaccagctcctacaattgccagccagccactgtctctgaggcccgaagcttgcaggcctgctgctggcggagccgtgcatacaagaggactggatttcgcctgcgacatctacatctgggcacctctggctggaacctgtggcgtgctgctgctgagcctggtcatcaccctgtattgccggagcaagagaagcagactgctgcacagcgactacatgaacatgacccctagacggcccggacctaccagaaagcactaccagccttacgctcctcctagagacttcgccgcctacagatccagagtgaagttcagcagatccgccgacgctcctgcctatcagcagggccaaaaccagctctacaacgagctgaacctggggagaagagaagagtacgacgtgctggacaagcggagaggcagagatcctgaaatgggcggcaagcccagacggaagaatcctcaagagggcctgtataatgagctacagaaagacaagatggcagaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 46 - protein, 43 aa, Mus sp.
TTTKPVLRTPSPVHPTGTSQPQRPEDCRPRGSVKGTGLDFACD

SEQ ID NO: 47 - DNA, 129 bp, Mus sp.
actactaccaagccagtgctgcgaactccctcacctgtgcaccctaccgggacatctcagccccagagaccagaagattgtcggccccgtggctcagtgaaggggaccggattggacttcgcctgtgat

SEQ ID NO: 48 - protein, 36 aa, Mus sp.
IEFMYPPPYLDNERSNGTIIHIKEKHLCHTQSSPKL

SEQ ID NO: 49 - DNA, 108 bp, Mus sp.
atcgagttcatgtaccctccaccttacctggacaacgagagaagcaacggcaccatcatccacatcaaagagaagcacctctgtcacacccagagcagccccaagctg

SEQ ID NO: 50 - protein, 26 aa, Mus sp.
IYIWAPLAGICVALLLSLIITLICYT

SEQ ID NO: 51 - DNA, 78 bp, Mus sp.
atttacatctgggcacccttggccggaatctgcgtggcccttctgctgtccttgatcatcactctcatctgctacaca

SEQ ID NO: 52 - protein, 27 aa, Mus sp.
FWALVVVAGVLFCYGLLVTVALCVIWT

SEQ ID NO: 53 - DNA, 81 bp, Mus sp.
ttttgggctcttgtggtggtggctggcgtgctgttttgttacggcctgctggtcacagtggccctgtgtgtgatttggacc

SEQ ID NO: 54 - protein, 41 aa, Mus sp.
NSRRNRGGQSDYMNMTPRRPGLTRKPYQPYAPARDFAAYRP

SEQ ID NO: 55 - DNA, 123 bp, Mus sp.
aacagcagaagaaacagaggcggacagagcgactacatgaacatgacccctagacggcctggcctgacaagaaagccctaccagccttacgctcccgccagagatttcgccgcctacagacct

SEQ ID NO: 56 - protein, 113 aa, Mus sp.
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 57 - DNA, 342 bp, Mus sp.
agagccaagttcagcagatccgccgaaacagccgccaacttgcaagatcctaaccagctgtacaacgagctgaacctggggagaagagaagagtacgacgtgctggaaaagaagagggccagagatccagagatgggcggcaagcagcagagaagaagaaaccctcaagagggcgtgtacaacgctctgcaaaaagacaagatggccgaggcctacagcgagatcggaactaagggcgaacgcagaagaggcaagggccacgatggactgtaccagggcctgagcacagccaccaaggacacatacgatgccctgcacatgcagacactggcccctagatag

SEQ ID NO: 58 - protein, 21 aa, Homo sapiens
MDSYLLMWGLLTFIMVPGCQA

SEQ ID NO: 59 - DNA, 63 bp, Homo sapiens
atggattcatacctgctgatgtggggactgctcacgttcatcatggtgcctggctgccaggca

SEQ ID NO: 60 - protein, 20 aa, Homo sapiens
METPAQLLFLLLLWLPDTTG

SEQ ID NO: 61 - DNA, 60 bp, Homo sapiens
atggaaaccccagcgcagcttctcttcctcctgctactctggctcccagataccaccgga

SEQ ID NO: 62 - DNA, 1188 bp, Homo sapiens
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 63 - DNA, 516 bp, Homo sapiens
tccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 64 - protein, 765 aa, Artificial Sequence
  Synthetic peptide
MALPVTALLLPLALLLHAARPSYPYDVPDYASQSVLTQPPSVSGAPGQRVTISCTGSSSNIGAGYDVHWYQQLPGTAPKLLIYGNNNRPSGVPDRFSGSKSGTSASLAITGLQAEDEADYYCQSYDTGLSAWVFGGGTKLTVLGGGGSGGGGSGGGGSQLQLQESGPGLVKPSETLSLTCTVSGGSITSSNYYWGWIRQPPGKGLEWIGSIYYSGNTYYNPSLKSRVTISVDTSKNQFSLKLSSVTAADTAVYYCAREWSPYESEGFDYWGQGTLVTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 65 - DNA, 2298 bp, Artificial Sequence
  Synthetic polynucleotide
atggctctgcctgtgacagctctgctgctgcctctggctctgcttcttcatgccgccagaccatcttacccctacgatgtgcctgattatgcatctcagagcgtgctgacacagcctccatctgtttctggcgcccctggccagagagtgaccatctcttgtactggcagcagctccaacattggagccggctatgacgtgcactggtaccagcagctgcctggcacagcccctaaactgctgatctacggcaacaacaacagacccagcggcgtgcccgacagattttctggctccaagagcggcacaagcgccagcctggctattactggactgcaggccgaggacgaggccgactactactgtcagagctacgacaccggcctgagcgcctgggtttttggtggaggcacaaaactgacagtgctgggaggcggaggttctggcggcggaggaagtggtggcggaggctcacagctgcagctacaggagagcggccccggcctggtgaaaccttctgagaccctgagcctgacgtgcaccgtgagcggcggcagcatcaccagcagcaactactactggggctggatacgacagccccctggaaagggcctagagtggatcggcagcatctactacagcggcaacacctactacaaccccagcctgaagagcagagtcaccatcagcgtggacaccagcaagaaccagttcagcctgaagctgagcagcgtgaccgccgccgacacagctgtttactattgtgccagagagtggagcccctacgagagcgagggcttcgactactggggccaggggaccctggtgaccgtttcttctacaacaacacctgctcctcggcctcctacaccagctcctacaattgccagccagccactgtctctgaggcccgaagcttgcaggcctgctgctggcggagccgtgcatacaagaggactggatttcgcctgcgacatctacatctgggcacctctggctggaacctgtggcgtgctgctgctgagcctggtcatcaccctgtattgccggagcaagagaagcagactgctgcacagcgactacatgaacatgacccctagacggcccggacctaccagaaagcactaccagccttacgctcctcctagagacttcgccgcctacagatccagagtgaagttcagcagatccgccgacgctcctgcctatcagcagggccaaaaccagctctacaacgagctgaacctggggagaagagaagagtacgacgtgctggacaagcggagaggcagagatcctgaaatgggcggcaagcccagacggaagaatcctcaagagggcctgtataatgagctacagaaagacaagatggcagaggcctacagcgagatcggaatgaagggcgagcgcagaagaggcaagggacacgatggactgtaccagggcctgagcaccgccaccaaggatacctatgatgccctgcacatgcaggccctgcctccaagagctagcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 66 - DNA, 744 bp, Artificial Sequence
  Synthetic polynucleotide
caatctgtcctgacccagccaccctctgttagtggagcacctggacagagagtgaccatcagttgtactgggagcagctccaacattggagccggctatgacgtccactggtaccagcagcttccaggcactgctcccaaactcctgatctacggcaacaacaatcgaccaagtggggtcccagacaggttcagtggttccaaatctgggacatcagcatctctggccattacaggcctccaggctgaagatgaagcagactactactgccagtcatatgacacagggctctctgcatgggtgtttggtggagggacaaagctgactgtgctgggaggcggaggttctggcggcggaggaagtggtggcggaggctcacagctgcagttgcaggagtctgggcctggacttgtcaaaccctctgaaacactgtcactgacttgcacagtctctggaggaagcatcacctcttcaaactactactggggctggatcagacagccaccagggaagggcctggagtggattggaagcatctactacagcgggaacacttactacaatccctctctgaaatcaagggtcaccatcagtgtggacacctccaagaaccagttctcactcaagctgagctcagtgacagctgcagacactgctgtttattattgtgcaagagagtggagcccctacgagtctgaaggctttgactactggggccaaggcactcttgtgaccgtgtccagc

SEQ ID NO: 67 - DNA, 135 bp, Homo sapiens
accactactcctgctcctcggcctcctacaccagctcctactatagccagtcagccactgagcctgaggcctgaggcctgtagaccagcagctggcggggctgttcatacaagaggcctggattttgcctgtgac

SEQ ID NO: 68 - DNA, 72 bp, Artificial Sequence
  CD8 transmembrane domain coding sequence
atctacatatgggcacctcttgctggtacatgtggagtgctgctgctgagcttggtcatcaccctctactgc

SEQ ID NO: 69 - DNA, 336 bp, Homo sapiens
agagtgaagttctcaagaagtgccgacgccccagcctatcagcaaggccagaaccagctgtataatgaattgaacctgggaaggagggaggaatatgatgttctggacaagaggagaggcagggaccctgaaatgggcgggaaaccacgaagaaagaacccccaggaaggactttacaatgagctccagaaagacaagatggctgaagcctattctgagatcggcatgaagggagagcgcagaagaggcaaagggcacgacggcctgtatcaagggctctccacagctaccaaagacacctatgatgctctgcacatgcaggctctgcctccccgc

SEQ ID NO: 70 - DNA, 123 bp, Homo sapiens
cggagcaaaagaagcagactcctccattctgactacatgaatatgacacccaggagaccaggaccaacaaggaagcactaccaaccttatgctcctcctcgggattttgccgcctatagatcc

SEQ ID NO: 71 - DNA, 744 bp, Artificial Sequence
  Synthetic polynucleotide
cagagtgtgctcacacaacctccatctgtttctggtgctcctgggcagagagtcaccatctcttgtactgggtcgtcctccaatattggagctggctatgatgtccactggtatcaacagctgcctgggacagcccctaaactcctcatctatggcaacaataacaggcccagtggtgtgcctgacagattttctgggtccaagtcaggcacaagtgcctcattggctattactggactgcaggctgaggatgaggcagactactactgtcagtcatatgacactggcctaagtgcctgggtttttggtggaggcacaaaactgacagtcctgggaggtggaggttctggcggtggagggagtggtggcggaggctcacaacttcagctacaagagagtggccctgggcttgtgaaaccttctgagaccctctctctgacgtgcacagtgtctggaggaagcatcaccagtagtaactactactggggctggatacgacagccccctggaaaggggctagagtggattggcagcatctattattcaggcaacacgtattataatcccagcctcaagagcagagtcaccatctctgtggacacgtcaaagaaccagttctctctaaagctgtccagtgtgactgctgcagacacagctgtttactattgtgccagagaatggtctccttatgagagtgagggctttgactactggggacagggaaccctggtgacagtctcttct

SEQ ID NO: 72 - protein, 6 aa, Artificial Sequence
  Synthetic peptide
GGSGGS

(19) United States
(12) Patent Application Publication
(10) Pub. No.: US 2026/0027152 A1
(43) Pub. Date: Jan. 29, 2026

(54) Antigen Binders Specific For IL-23R And Uses Thereof

(71) Applicant: Sangamo Therapeutics, Inc., Richmond, CA (US)

(72) Inventors: Tobias ABEL, Le Cannet (FR); Maurus DE LA ROSA, Antibes (FR); David FENARD, Antibes (FR); Julie GERTNER-DARDENNE, Grasse (FR)

(73) Assignee: Sangamo Therapeutics, Inc., Richmond, CA (US)

(21) Appl. No.: 18/572,962

(22) PCT Filed: Jun. 24, 2022
(86) PCT No.: PCT/US2022/035028

§ 371 Date: Dec. 21, 2023

Related U.S. Application Data
(60) Provisional application No. 63/214,950, filed on Jun. 25, 2021.

(30) Foreign Application Priority Data
Jun. 25, 2021 (EP) 21181801.8

(51) Int. Cl. A61K 35/17; A61K 40/11; A61K 40/31; A61K 40/42; A61P 37/06; C07K 14/705; C07K 14/725; C07K 16/28; C12N 15/86

(52) U.S. Cl. CPC A61K 35/17; A61K 40/11; A61K 40/31; A61K 40/4217; A61P 37/06; C07K 14/7051; C07K 14/70517; C07K 14/70521; C07K 16/2866; C12N 15/86; A61K 2239/13; A61K 2239/17; A61K 2239/21; A61K 2239/22; C07K 2317/24; C07K 2317/565; C07K 2317/622; C07K 2317/92; C12N 2740/15043

(57) ABSTRACT

IL23R overexpression has been described as a common feature of pathogenic inflammatory cells involved in the onset and maintenance of autoimmune diseases and chronic inflammation. The present disclosure relates to a novel antigen binder directed to IL-23R, to compositions that comprise said antigen binder, and to the use of said antigen binder. The present disclosure further relates to fusion proteins comprising said antigen binder, such as, for example, chimeric antigen receptors.

### CROSS-REFERENCE TO RELATED APPLICATIONS

This application claims priority from U.S. Patent Application 63/214,950 and European Patent Application EP 21181801.8, both filed on Jun. 25, 2021. The disclosures of both priority applications are incorporated by reference herein in their entirety.

### SEQUENCE LISTING

The instant application contains a Sequence Listing that has been submitted electronically in ASCII format and is hereby incorporated by reference in its entirety. Said ASCII copy, created on Jun. 20, 2025, is named 5724-107US_ST25.txt and is 47,389 bytes in size.

### BACKGROUND OF THE INVENTION

Interleukin-23 (IL-23), a member of the IL-12 cytokine family, is composed of two subunits, p19 and p40. The receptor for IL-23 (IL-23R) consists of an IL-23Rα subunit in complex with an IL-12β1 subunit, which is a common subunit for the IL-12 receptor and interacts with tyrosine kinase 2 (Tyk2).

IL-23R is mainly expressed on immune cells, in particular T cells (e.g., Th17 and γδ T cells), macrophages, dendritic cells and NK cells (Duvallet et al., Ann Med. (2011) 43(7):503-11). The IL-23/IL-23R signaling pathway has been described as critical for promoting the proliferation and the differentiation of IL-17-secreting immune cells, in particular CD4⁺ Th17 cells and γδ T cells. IL23R overexpression has been described as a common feature of pathogenic inflammatory cells involved in the onset and maintenance of autoimmune diseases and chronic inflammation. Cell surface expression of IL-23R is induced by IL-23 exposure, and depends on inflammation levels.

### SUMMARY OF THE INVENTION

The inventors herein have developed a novel tool to activate immune cells at inflammation sites, based on the binding of IL-23R overexpressed at inflammation sites. More specifically, the inventors disclose herein a novel antibody capable of binding to IL-23R. In particular, the inventors disclose herein a novel antibody capable of binding to IL-23R with high affinity. Advantageously, IL-23R-binding antibodies of the invention have been found to be capable of binding to both a human and a mouse IL-23R. Inclusion of the antigen-binding portion of said antibody in chimeric antigen receptors (CAR) expressed on the cell surface of immune cells allows activation of these cells at inflammation sites upon binding to IL-23R. The inventors disclose a stable signaling of said antigen-binding portion and a low background activation. Without being bound by theory, the inventors hypothesize that the advantage of the present CAR may in part reside in its ability to cause less tonic signaling in the immune cells (e.g., T cells) expressing it, thereby improving the cells' therapeutic potential. These engineered immune cells would thus be a valuable therapeutic tool for treating autoimmune and/or inflammatory diseases or disorders.

In one aspect, the present disclosure provides an isolated anti-IL-23 receptor (IL-23R) antibody or antigen-binding fragment thereof, wherein the heavy chain variable region (VH) of the antibody or fragment comprises complementary-determining regions (HCDRs) 1-3 comprising SEQ ID NOs: 1-3, respectively; or any CDR having an amino acid sequence that shares at least about 90% of identity with one of SEQ ID NOs: 1-3; and the light chain variable region (VL) of the antibody or fragment comprises complementary-determining regions (LCDRs) 1-3 comprising SEQ ID NOs: 4-6, respectively; or any CDR having an amino acid sequence that shares at least about 90% of identity with one of SEQ ID NOs: 4-6. In one aspect, the present disclosure provides an isolated anti-IL-23 receptor (IL-23R) antibody or antigen-binding fragment thereof, wherein the heavy chain variable region (VH) of the antibody or fragment comprises complementary-determining regions (HCDRs) 1-3 having an amino acid sequence that share at least about 90% of identity with SEQ ID NOs: 1-3 respectively; and the light chain variable region (VL) of the antibody or fragment comprises complementary-determining regions (LCDRs) 1-3 having an amino acid sequence that share at least about 90% of identity with SEQ ID NOs: 4-6 respectively. In one aspect, the present disclosure provides an isolated anti-IL-23 receptor (IL-23R) antibody or antigen-binding fragment thereof, wherein, the heavy chain variable region (VH) of the antibody or fragment comprises complementary-determining regions (HCDRs) 1-3 comprising SEQ ID NOs: 1-3, respectively; and the light chain variable region (VL) of the antibody or fragment comprises complementary-determining regions (LCDRs) 1-3 comprising SEQ ID NOs: 4-6, respectively. In some embodiments, said antibody or antigen-binding fragment is capable of binding mouse and human IL-23R. In some embodiments, said antibody or antigen-binding fragment is capable of binding to a human IL-23R alpha subunit with an EC50 of less than 40 nM. In some embodiments, said antibody or antigen-binding fragment is capable of binding to a mouse IL-23R alpha subunit with an EC50 of less than 60 nM. In some embodiments, said VH comprises SEQ ID NO: 7 or an amino acid sequence at least about 90% identical thereto, and said VL comprises SEQ ID NO: 8 or any amino acid sequence at least about 90% of identical thereto. In some embodiments, said VH comprises SEQ ID NO: 7, and said VL comprises SEQ ID NO: 8. In certain embodiments, said antibody or antigen-binding fragment is an scFv comprising SEQ ID NO: 15 or any amino acid sequence at least about 95% identical thereto. In some embodiments, said antibody or antigen-binding fragment is an scFv comprising SEQ ID NO: 15.

The present disclosure also provides a chimeric antigen receptor (CAR) comprising an extracellular domain comprising an anti-IL-23R antibody or antigen-binding fragment thereof as described herein; a transmembrane domain; and a cytoplasmic domain comprising an intracellular signaling domain. In some embodiments, the CAR further comprises a leader sequence. In some embodiments, the extracellular domain comprises an scFv comprising SEQ ID NO: 15. In some embodiments, the intracellular signaling domain comprises a human CD28 costimulatory signaling domain (e.g., comprising SEQ ID NO: 32 or an amino acid sequence at least about 90% identical thereto), and/or a human CD3 zeta domain (e.g., comprising SEQ ID NO: 30 or an amino acid sequence at least about 90% identical thereto). In some embodiments, the transmembrane domain is derived from human CD8 (e.g., comprising SEQ ID NO: 22 or an amino acid sequence at least about 90% identical thereto). In certain embodiments, the CAR comprises said extracellular domain, said transmembrane domain, and/or said intracellular signaling domain. In some embodiments, the leader sequence comprises an amino acid sequence derived from a CD8 leader sequence, optionally comprising SEQ ID NO: 40 or an amino acid sequence at least about 95% (e.g., about 96%, 97%, 98%, or 99%) identical thereto. In some embodiments, the leader sequence comprises an amino acid sequence derived from a CD25 leader sequence, optionally comprising SEQ ID NO: 58 or an amino acid sequence at least about 95% (e.g., about 96%, 97%, 98%, or 99%) identical thereto. In particular embodiments, the CAR comprises an anti-IL-23R scFv (e.g., comprising SEQ ID NO: 15), a hinge domain derived from human CD8 (e.g., comprising SEQ ID NO: 20), a transmembrane domain derived from human CD8 (e.g., comprising SEQ ID NO: 22), an intracellular signaling domain comprising a human CD28 costimulatory signaling domain (e.g., comprising SEQ ID NO: 32), and a human CD3 zeta domain (e.g., comprising SEQ ID NO: 30). The CAR may also comprise a tag and/or a leader sequence. In particular embodiments, the CAR comprises an anti-IL-23R scFv (e.g., comprising SEQ ID NO: 15), a hinge domain derived from human CD8 (e.g., comprising SEQ ID NO: 20), a transmembrane domain derived from human CD8 (e.g., comprising SEQ ID NO: 22), an intracellular signaling domain comprising a human CD28 costimulatory signaling domain (e.g., comprising SEQ ID NO: 32), and a human CD3 zeta domain (e.g., comprising SEQ ID NO: 30), and a leader sequence derived from CD8 (e.g., comprising SEQ ID NO: 40). In particular embodiments, the CAR comprises an anti-IL-23R scFv (e.g., comprising SEQ ID NO: 15), a hinge domain derived from human CD8 (e.g., comprising SEQ ID NO: 20), a transmembrane domain derived from human CD8 (e.g., comprising SEQ ID NO: 22), an intracellular signaling domain comprising a human CD28 costimulatory signaling domain (e.g., comprising SEQ ID NO: 32), and a human CD3 zeta domain (e.g., comprising SEQ ID NO: 30), and a leader sequence derived from CD25 (e.g., comprising SEQ ID NO: 58).

In one aspect, the present disclosure provides a nucleic acid molecule encoding an antibody or antigen-binding fragment as described herein, or a CAR as described herein. The present disclosure also provides a vector (e.g., an expression vector) comprising said nucleic acid molecule. Further, the present disclosure provides a cell comprising said nucleic acid molecule or vector as described herein. Further, the present disclosure provides an immune cell comprising said nucleic acid molecule or vector as described herein.

The present disclosure also provides a composition comprising a cell as described herein. The present disclosure also provides a composition comprising an immune cell as described herein. Also provided is the cell or population of cells as described herein, for use as a medicament. Also provided is the immune cell or a population of immune cells as described herein, for use as a medicament. In some embodiments, the cell or population of immune cells is for use in treating a disease or disorder mediated by IL-23R-expressing cells in a subject in need thereof. In some embodiments, the immune cell or population of immune cells is for use in treating a disease or disorder mediated by IL-23R-expressing cells in a subject in need thereof. The present disclosure also provides a method for treating a disorder or disease in a subject in need thereof, wherein the method comprises administering to said patient a cell as described herein, or a population of cells as described herein. The present disclosure also provides a method for treating a disorder or disease in a subject in need thereof, wherein the method comprises administering to said patient an immune cell as described herein, or a population of immune cells as described herein. The disease or disorder may be a disease or disorder mediated by IL-23R-expressing cells in the subject in need thereof. The disease or disorder may be an autoimmune or inflammatory disease or disorder. In certain embodiments, said disease or disorder is an autoimmune or inflammatory disease or disorder. The disease or disorder may be selected from, for example, inflammatory bowel diseases (e.g., Crohn's disease or ulcerative colitis), lupus (e.g., systemic lupus erythematosus), arthritis (e.g., rheumatoid arthritis or juvenile idiopathic arthritis), Sjogren's syndrome, systemic sclerosis, multiple sclerosis, ankylosing spondylitis, type 1 diabetes, autoimmune thyroid disorders, myasthenia gravis, psoriasis, psoriatic arthritis, skin diseases and uveitis. In particular embodiments, the disease is Crohn's disease.

### BRIEF DESCRIPTION OF THE DRAWINGS

FIG. 1 represents a schematic view of an anti-IL-23R chimeric antigen receptor (CAR) construct (CAR #2) of the present disclosure. The anti-IL23R CAR comprises an scFv directed against the human/mouse (hm) IL-23R (αIL-23R; IL23RLamS4-G3), a hinge domain (CD8 linker), a transmembrane domain derived from human CD8 (CD8 TM), an intracellular signaling domain derived from human CD28 (CD28) and CD3 zeta (CD3ζ or CD3Z). The CAR construct further comprises a GFP coding sequence and a HA-tag coding sequence. This construct is compared to the CAR #1 construct, derived from the scFv 14-11-D07 targeting the IL-23R as well.

FIGS. 2A and 2B are graphs monitoring the regulatory T cell (Treg) phenotype of non-transduced cells, and cells transduced with CAR #2 or CAR #1 at the end of the first cycle of expansion. Treg cells were labeled with antibodies directed against human CD4, CD25, CD127, and CTLA-4. For detection of FOXP3 and Helios transcription factors, an intra-nuclear labeling was performed (FIG. 2A). Helios and FOXP3 were also evaluated after 11 days of CAR engagement (FIG. 2B). Error bars represent mean±SEM from three independent experiments including five Treg donors in total.

FIG. 3 is a graph showing Treg activation status (measured by CD69 expression, gated on GFP expression) either in the absence of activation (noAct) or following 24 h stimulation through the CAR (via addition of IL-23R coated beads; IL-23R) or through the TCR (via beads coated with anti-CD3 and anti-CD28; 3/28) from three independent experiments including five Treg donors in total.

FIG. 4 is a combination of graphs showing that Treg cells expressing CAR #2 exhibit efficient CAR-mediated suppressive activity. Contact-dependent suppression mediated by IL-23R-CAR in the absence of any activation (dotted curve) or after IL23R-induced CAR activation (black curve) or after TCR-induced activation (grey curve) was evaluated by measuring the proliferation of conventional T cells (Tconv) using flow cytometry. Error bars represent mean±SEM from three independent experiments including four Treg donors in total.

FIG. 5 represents a schematic view of a mouse version of the anti-IL-23R chimeric antigen receptor (CAR) construct (mCAR #2b) of the present disclosure. This mouse version comprises an scFv directed against human/mouse IL-23R (αIL-23R; IL23RLamS4-G3, which is human/mouse cross-reactive), a CD8 transmembrane domain derived from the mouse hinge (mCD8 TM), an intracellular domain derived from mouse CD28 (mCD28), and mouse CD3 zeta (mCD3Z). This construct is compared to mouse CAR #2a (mCAR #2a), which corresponds to mCAR #2b but with a mouse CD28 transmembrane domain (mCD28 TM); and mouse CAR #1 (mCAR #1), derived from a published scFv (14-11-D07) targeting IL-23R.

FIG. 6 depicts a schematic showing the design for the short model of dextran sodium sulfate (DSS)-induced inflammatory bowel disease (IBD) (Panel A); and a set of graphs showing the measurement of CTLA-4 positive Treg cells in mesenteric lymph node (left), colon (middle), and spleen (right) (Panel B).

FIG. 7 depicts a schematic showing the design for an efficacy model of DSS-induced IBD (Panel A); and a graph showing the measurement of the disease activity index of three groups of mice receiving control treatment (saline), non-transduced Treg cells (“Poly Treg”), or CAR-Treg cells (“CAR #2b Treg”) (Panel B).

FIG. 8 depicts a graph showing consistent transduction efficiency (around 40-50%) for CAR #2, CAR #3, CAR #4, CAR #5, CAR #6, and CAR #7. Results are the mean±SEM from 9 different Treg donors.

FIG. 9 depicts graphs showing consistent viability and fold expansion of the CAR-Treg for CAR #2, CAR #3, CAR #4, CAR #5, CAR #6, and CAR #7. Results are the mean±SEM from 9 different Treg donors.

FIG. 10 depicts graphs showing activation of the CAR-Treg for CAR #2, CAR #3, CAR #4, CAR #5, CAR #6, and CAR #7. CAR #3 showed the lowest background of activation and the highest signal versus noise after CAR activation. Activation of CAR Treg was assessed by flow cytometry by measuring the CD69 expression at cell surface of CAR Treg 24 h after inducing activation either through the TCR using anti-CD3/anti-CD28 coated beads (in grey) or through the CAR using either IL-23R coated beads or Jurkat expressing low level of IL-23R at cell surface (Jurkat 572) or Jurkat expressing high level of IL-23R at cell surface (Jurkat 573). Results are the mean±SEM from 6 Treg donors. Two-way Anova and Sidak's multiple comparisons test. *=0.05 **=0.01 ***=0.001 and ****=0.0001.

FIG. 11 depicts graphs showing CAR-mediated suppressive activity for CAR #2, CAR #3, CAR #4, CAR #5, CAR #6, and CAR #7. Suppression mediated by CAR Treg was assessed by flow cytometry by measuring the inhibition of Tconv proliferation after 3 Days of coculture. CAR Treg are activated 24 h before the coculture either through the TCR using anti-CD3/anti-CD28 coated beads (black circle) or through the CAR using either IL-23R coated beads (black square) or the 2 Jurkat cell lines expressing high (Jurkat 573, grey square) or low (Jurkat 572, white square) level of IL-23R at cell surface. Results are the mean±SEM from 4 independent experiments including 6 Treg donors. Two-way Anova and Sidak's multiple comparisons test. *=0.05 **=0.01.

FIG. 12 shows the area under the curve (AUC) for CAR-mediated suppressive activity of CAR #2 and CAR #3. The AUC calculation highlights that only the CAR #3 shown the same CAR-mediated suppression when Treg are engaged with Jurkat expressing high level of IL-23R at cell surface (Jurkat 573) whereas lower CAR-mediated suppression when CAR Treg was engaged with Jurkat expressing low level (Jurkat 572). This exemplary result supports a reduced off target effect with CAR #3.

FIG. 13 depicts a graph showing the phenotypic stability of the Treg phenotype. Main Treg Markers (CD4, CD25, CD127, FoxP3, Helios and CTLA-4) were measured by flow cytometry. Results are the mean±SEM from 9 different Treg donors.

FIG. 14 depicts affinity (EC50) results of CAR #1 and CAR #2 binding to mouse and human IL23R.

FIG. 15 shows 4 different constructs (CAR #8, CAR #9, CAR #10, and CAR #11) generated with different expression cassettes (PGK or EF1a promoters, +/−WPRE Mut6).

FIG. 16 depicts graphs showing the stability of scFv signaling with various CAR constructs of the present disclosure.

### DETAILED DESCRIPTION OF THE INVENTION

### Definitions

In the present disclosure, the following terms have the following meanings:

“About” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

“Affinity” is used to define the strength of an antibody-antigen complex. Affinity measures the strength of interaction between an epitope and an antigen binding site on an antibody. It may be expressed by an affinity constant Kₐ or by a dissociation constant K_{D}. An antibody is said to specifically bind to an antigen when the K_{D} is ≤1 μM, preferably ≤100 nM or ≤10 nM. K_{D} can be measured, e.g., by surface plasmon resonance (SPR) (BIAcore™) or Bio-Layer Interferometry, for example, using the IBIS MX96 SPR system from IBIS Technologies, the ProteOn™ XPR36 SPR system from Bio-Rad, or the Octet™ system from ForteBio.

“Antibody” or “immunoglobulin” as used herein, refers to a tetramer comprising two heavy chains and two light chains interconnected by disulfide bonds. Each light chain is composed of a light chain variable domain or region (VL) and a light chain constant region (CL) and can be a kappa (κ) light chain or a lambda (λ) light chain. Each heavy chain is comprised of a heavy chain variable domain or region (VH) and a heavy chain constant region (CH). Based on the amino acid sequence of the CH, antibodies can be assigned to different isotypes: IgA, IgD, IgE, IgG, or IgM. The IgG and IgA isotypes are further divided into subclasses: IgG1, IgG2, IgG3, IgG4, IgA1, and IgA2. The pairing of a VH and a VL forms a single antigen-binding site. In one embodiment, the anti-IL-23R antibody of the present disclosure is an IgG antibody.

“Antigen-binding fragment”, as used herein, refers to a part or region, or a derivative of an antibody that comprises fewer amino acid residues than the whole antibody and yet remains capable of binding to the antigen (e.g., IL-23R) of the whole antibody. Antigen-binding fragments encompasses, without any limitation, single chain antibodies, Fv (e.g., scFv), Fab, Fab′, Fab′-SH, F(ab)′₂, Fd, defucosylated antibodies, diabodies, triabodies and tetrabodies.

“Chimeric antigen receptor” or “CAR” refers to a protein, such as a fusion protein, which when expressed in an immune cell, provides the cell with specificity for a target ligand and with intracellular signal generation. In some embodiments, the CAR comprises a set of polypeptides that include a dimerization switch that, upon the presence of a dimerization molecule, can couple the polypeptides to one another, e.g., can couple a ligand-binding domain to an intracellular signaling domain. In one embodiment, the CAR comprises an optional leader sequence at the N-terminus, wherein the leader sequence is cleaved during cellular processing and localization of the chimeric antigen receptor to the cellular membrane.

Complementarity-determining region” or “CDR” means the non-contiguous antigen combining sites found within the heavy chain variable region (VH) and the light chain variable region (VL). The precise amino acid sequence boundaries of a given CDR can be determined using any of a number of well-known schemes, including those described by Kabat et al., “Sequences of Proteins of Immunological Interest,” 5th Ed. (1991) Public Health Service, National Institutes of Health, Bethesda, MD (“Kabat” numbering scheme), Al-Lazikani et al., JMB (1997) 273:927-948 (“Chothia” numbering scheme), or a combination thereof. More recently, a universal numbering system has been developed and widely adopted, ImMunoGeneTics (IMGT) Information System® (Lefranc et al., Nucleic Acids Res. (1999) 27:209-212). In one embodiment, the CDR boundaries herein are defined in accordance with Kabat et al. (1991).

“Costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. A costimulatory signaling domain can be the intracellular portion of a costimulatory molecule. A costimulatory molecule can be represented in the following protein families: TNF receptor proteins, immunoglobulin-like proteins, cytokine receptors, integrins, signaling lymphocytic activation molecules (SLAM proteins), and activating NK cell receptors.

“Epitope” refers to a specific arrangement of amino acids located on a protein or proteins to which an antibody or antigen-binding fragment thereof binds. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

“Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Fv” is the minimum antibody fragment that contains a complete antigen-recognition and -binding site. This fragment consists of a dimer of one heavy- and one light-chain variable region domain in tight, non-covalent association. From the folding of these two domains emanate six hypervariable loops (three loops each from the H and L chain) that contribute to the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Identity” or “identical,” when used herein to describe the relationship between two or more amino acid sequences, or between two or more nucleic acid sequences, refers to the degree of sequence relatedness between the compared sequences. “Identity” measures the percentage of identical matches between the smaller of two or more sequences with gap alignments (if any) addressed by a particular mathematical model or computer program (i.e., “algorithms”). Identity of related amino acid sequences or nucleic acid sequences can be readily calculated by known methods. Such methods include, but are not limited to, those described in Lesk A. M. (1988). Computational molecular biology: Sources and methods for sequence analysis. New York, NY: Oxford University Press; Smith D. W. (1993). Biocomputing: Informatics and genome projects. San Diego, CA: Academic Press; Griffin A. M. & Griffin H. G. (1994). Computer analysis of sequence data, Part 1. Totowa, NJ: Humana Press; von Heijne G. (1987). Sequence analysis in molecular biology: treasure trove or trivial pursuit. San Diego, CA: Academic press; Gribskov M. R. & Devereux J. (1991). Sequence analysis primer. New York, NY: Stockton Press; Carrillo et al., SIAM J Appl Math. (1988) 48(5):1073-82. Preferred methods for determining identity are designed to give the largest match between the sequences tested. Methods of determining identity are described in publicly available computer programs. Preferred computer program methods for determining identity between two sequences include the GCG program package, including GAP (Genetics Computer Group, University of Wisconsin, Madison, WI; Devereux et al., Nucleic Acids Res. (1984) 12(1 Pt 1):387-95), BLASTP, BLASTN, and FASTA (Altschul et al., J Mol Biol. (1990) 215(3):403-10). The BLASTX program is publicly available from the National Center for Biotechnology Information (NCBI) and other sources (BLAST Manual, Altschul et al. NCB/NLM/NIH Bethesda, Md. 20894). The well-known Smith Waterman algorithm may also be used to determine identity.

“Intracellular signaling domain” as used herein, refers to an intracellular portion of a molecule. The intracellular signaling domain generates a signal that promotes an immune effector function of the chimeric receptor containing cell. Examples of immune effector function in a chimeric receptor-T cell may include cytolytic activity, suppressive activity, regulatory activity and helper activity, including the secretion of cytokines.

“Subject” is intended to include living organisms in which an immune response can be elicited (e.g., mammals, human). In one embodiment, a subject may be a “patient”, i.e., a warm-blooded animal, more preferably a human, who/which is awaiting the receipt of, or is receiving medical care or was/is/will be the object of a medical procedure, or is monitored for the development of the targeted disease or condition, such as, for example, an inflammatory or autoimmune condition. In one embodiment, the subject is an adult (for example a subject above the age of 18). In another embodiment, the subject is a child (for example a subject below the age of 18). In one embodiment, the subject is a male. In another embodiment, the subject is a female. In one embodiment, the subject is affected, preferably is diagnosed, with an autoimmune and/or inflammatory disease or disorder. In one embodiment, the subject is at risk of developing an autoimmune and/or inflammatory disease or disorder. Examples of risks factor include, but are not limited to, genetic predisposition, or familial history of an autoimmune and/or inflammatory disease or disorder.

“Single-chain Fv”, also abbreviated as “sFv” or “scFv”, refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. In one embodiment, the present antigen-binding fragment is a single chain Fv (scFv).

“Therapeutically effective amount” refers to the level or amount of an antibody as described herein that is aimed at, without causing significant negative or adverse side effects to the target, (1) delaying or preventing the onset of a disease, disorder, or condition; (2) slowing down or stopping the progression, aggravation, or deterioration of one or more symptoms of the disease, disorder, or condition; (3) bringing about ameliorations of the symptoms of the disease, disorder, or condition; (4) reducing the severity or incidence of the disease, disorder, or condition; or (5) curing the disease, disorder, or condition. A therapeutically effective amount may be administered prior to the onset of the disease, disorder, or condition, for a prophylactic or preventive action. Alternatively or additionally, the therapeutically effective amount may be administered after initiation of the disease, disorder, or condition, for a therapeutic action.

“Treating” or “treatment” or “alleviation” refers to both therapeutic treatment and prophylactic or preventative measures, wherein the objective is to prevent or slow down (lessen) the targeted pathologic condition or disorder. Those in need of treatment include those already with the disorder as well as those prone to have the disorder or those in whom the disorder is to be prevented. In one embodiment, a subject is successfully “treated” for a disease or disorder if, after receiving a therapeutic amount of an antibody or of a cell according to the present disclosure, the subject shows at least one of the following: reduction in the number or percentage of pathogenic cells; relief to some extent of one or more of the symptoms associated with the disease or disorder to be treated; reduced morbidity and mortality; and improvement in quality-of-life issues. The above parameters for assessing successful treatment and improvement in the disease are readily measurable by routine procedures familiar to a physician.

“Zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain, or functional derivatives thereof, that are sufficient to functionally transmit an initial signal necessary for T cell activation. In one embodiment, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

### I. Antibodies and Antigen-Binding Fragments

The present disclosure first relates to an isolated monoclonal antibody or antigen-binding fragment thereof, wherein said antibody or antigen-binding fragment thereof binds to at least one IL-23R (e.g., the IL-23R alpha subunit).

An “isolated antibody”, as used herein, is intended to refer to an antibody that is substantially free of other antibodies having different antigenic specificities (e.g., an isolated antibody that specifically binds IL-23R is substantially free of antibodies that specifically bind antigens other than IL-23R). An isolated antibody that specifically binds IL-23R may, however, have cross-reactivity to other antigens, such as IL-23R molecules from other species. Moreover, an isolated antibody may be substantially free of other cellular material and/or chemicals, in particular those that would interfere with therapeutic uses of the antibody, including without limitation, enzymes, hormones, and other proteinaceous or non-proteinaceous components. The isolated antibody herein may be an IgG antibody, such as an IgG1, IgG2, or IgG4 antibody.

### I.1 Antigen Specificity and Affinity

In one embodiment, the present antibody or antigen-binding fragment thereof recognizes and is capable to bind to an IL-23R expressed on the cell surface.

In another embodiment, the present antibody or antigen-binding fragment thereof recognizes and is capable of binding to a soluble IL-23R (i.e., not membrane bound).

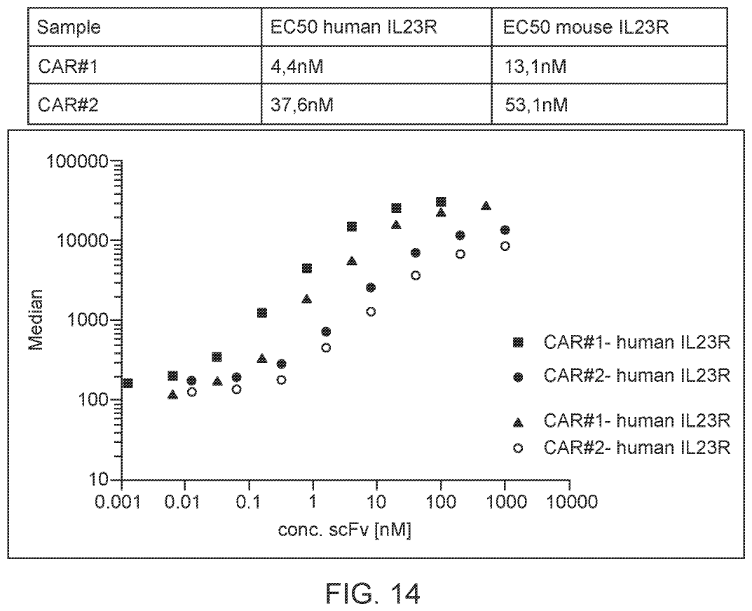

Advantageously, the present IL-23R-binding antibody or antigen-binding fragment has been found to be capable of binding to both a human and a mouse IL-23R, in particular with high affinity (FIG. 14). This cross-reactivity is beneficial for extrapolating results from preclinical studies in mice to human clinical studies for the drug approval process.

In an embodiment, the present antibody or antigen-binding fragment, e.g. a scFv according to the present invention, is able to bind both mouse and human IL-23R (FIG. 14). Thus, cross-reactivity of the scFv with mouse and human IL-23R is an advantageous feature.

In one embodiment, the present antibody or antigen-binding fragment thereof recognizes and binds to a human IL-23R alpha subunit. Preferably, the present antibody or antigen-binding fragment thereof binds to a human IL-23R alpha subunit with high affinity (FIG. 14). Human IL-23R alpha subunit is a protein encoded by a 2.8 kb long mRNA comprising 11 exons (Genbank accession number: NM_144701).

In one embodiment, the present antibody or antigen-binding fragment thereof recognizes and is capable of binding to an IL-23R variant, such as a variant of a human IL-23R.

In one embodiment, a variant of IL-23R refers to a modified IL-23R wherein 1, 2, 3, 4, 5, 6, 7, 8, 9, 10, or more amino acids are deleted, added or substituted as compared to the original (wildtype) IL-23R.

Splice variants of human IL-23R have been previously identified (Kan et al, Genes and Immunity (2008) 9(7):631-639). In particular, 24 different isoforms of IL-23R have been described: isoform_v1 (encoded by a mRNA having the Genbank accession number AM990313), isoform_v2 (encoded by a mRNA having the Genbank accession number AM990314), isoform_v3 (encoded by a mRNA having the Genbank accession number AM990315), isoform_v4 (encoded by a mRNA having the Genbank accession number AM990316), isoform_v5 (encoded by a mRNA having the Genbank accession number AM990317), isoform_v6 (encoded by a mRNA having the Genbank accession number AM990318), isoform_v7 (encoded by a mRNA having the Genbank accession number AM990319), isoform_v8 (encoded by a mRNA having the Genbank accession number AM990320), isoform_v9 (encoded by a mRNA having the Genbank accession number AM990321), isoform_v10 (encoded by a mRNA having the Genbank accession number AM990322), isoform_v11 (encoded by a mRNA having the Genbank accession number AM990323), isoform_v12 (encoded by a mRNA having the Genbank accession number AM990324), isoform_v13 (encoded by a mRNA having the Genbank accession number AM990325), isoform_v14 (encoded by a mRNA having the Genbank accession number AM990326), isoform_v15 (encoded by a mRNA having the Genbank accession number AM990327), isoform_v16 (encoded by a mRNA having the Genbank accession number AM990328), isoform_v17 (encoded by a mRNA having the Genbank accession number AM990329), isoform_v18 (encoded by a mRNA having the Genbank accession number AM990330), isoform_v19 (encoded by a mRNA having the Genbank accession number AM990331), isoform_v20 (encoded by a mRNA having the Genbank accession number AM990332), isoform_v21 (encoded by a mRNA having the Genbank accession number AM990333), isoform_v22 (encoded by a mRNA having the Genbank accession number AM990334), isoform_v23 (encoded by a mRNA having the Genbank accession number AM990335) and isoform_v24 (encoded by a mRNA having the Genbank accession number AM990336).

Therefore, in one embodiment, the present antibody or antigen-binding fragment thereof recognizes and is capable of binding to a splice variant of human IL-23R selected from the group comprising isoform_v1, isoform_v2, isoform_v3, isoform_v4, isoform_v5, isoform_v6, isoform_v7, isoform_v8, isoform_v9, isoform_v10, isoform_v11, isoform_v12, isoform_v13, isoform_v14, isoform_v15, isoform_v16, isoform_v17, isoform_v18, isoform_v19, isoform_v20, isoform_v21, isoform_v22, isoform_v23, and isoform_v24.

Moreover, single nucleotide polymorphisms in the alpha subunit of human IL-23R have been previously described (Kan et al, supra; and Sivanesan et al., J Biol Chem. (2016) 291(16):8673-85).

In one embodiment, the present antibody or antigen-binding fragment thereof recognizes and is capable of binding to a human IL-23R variant comprising a single nucleotide polymorphism (SNP) in the alpha subunit, wherein said SNP is selected from the group comprising R381Q, G149R, V362I, and combinations thereof.

In one embodiment, the present antibody or antigen-binding fragment thereof recognizes and binds to a mouse IL-23R alpha subunit. Preferably, the present antibody or antigen-binding fragment thereof binds to a mouse IL-23R alpha subunit with high affinity (FIG. 14). Mouse IL-23R alpha subunit is a protein encoded by a 2.5 kb long mRNA comprising 11 exons (Genbank accession number: NM_144548).

Splice variants of mouse IL-23R have been previously identified. In particular, 7 different isoforms of IL-23R have been described: Isoform 1 (Uniprot Q5VWK5-1; NM_144701.3), Isoform 2 (Uniprot Q5VWK5-2; XP_005270573), Isoform 3 (Uniprot Q5VWK5-3), Isoform 4, (Uniprot Q5VWK5-4), and Isoform 5 (Uniprot Q5VWK5-5) (Zhang, et al., Immunogenetics 57:934-943(2006)), as well as Isoform 6, (Uniprot Q5VWK5-6; XP_005270574) and Isoform 7 (Uniprot Q5VWK5-7) (Genome Res. 14:2121-2127(2004)). In some embodiments, the anti-IL23R antibody or antigen-binding fragments of the present disclosure thereof binds to one or more of isoforms 1-7. In some embodiments, the anti-IL23R antibody or antigen-binding fragments of the present disclosure binds to at least one of isoform 1 and isoform 3. In some embodiments, the anti-IL23R antibody or antigen-binding fragments of the present disclosure binds to at least one of isoform 4 and isoform 6.

### I.2.1 CDR Sequences

In one embodiment, the CDR are determined in accordance to the Kabat CDR definition system.

In one embodiment, the heavy chain of the present antibody or antigen-binding fragment comprises at least one, preferably at least two, more preferably all three, of the following heavy chain CDRs (HCDRs):

HCDR1:
(SEQ ID NO: 1)
SSNYYWG
HCDR2:
(SEQ ID NO: 2)
GSIYYSGNTYYNPSL
HCDR3:
(SEQ ID NO: 3)
REWSPYESEGFDY

In one particular embodiment, the present antibody or antigen-binding fragment comprises all of SEQ ID NOs: 1-3. In one embodiment, any of HCDR1, HCDR2 and/or HCDR3 may comprise 1, 2, 3, or more amino acid modifications (e.g., substitutions) as compared to SEQ ID NOs: 1-3, respectively. In one embodiment, any of HCDR1, HCDR2 and/or HCDR3 has an amino acid sequence that shares at least 90%, 95%, 96%, 97%, 98%, 99% or more of identity with SEQ ID NOs: 1-3, respectively.

In one embodiment, the light chain of the present antibody or antigen-binding fragment comprises at least one, preferably at least two, more preferably all three, of the following light chain CDRs (LCDRs):

LCDR1:
(SEQ ID NO: 4)
TGSSSNIGAGYDVH
LCDR2:
(SEQ ID NO: 5)
GNNNRPS
LCDR3:
(SEQ ID NO: 6)
QSYDTGLSAW

In one particular embodiment, the present antibody or antigen-binding fragment comprises all of SEQ ID NOs: 4-6. In one embodiment, any of LCDR1, LCDR2 and/or LCDR3 may comprise 1, 2, 3, 4, 5, or more amino acid modifications (e.g., substitutions) as compared to SEQ ID NOs: 4-6, respectively. In one embodiment, any of LCDR1, LCDR2 and/or LCDR3 has an amino acid sequence that shares at least 90%, 95%, 96%, 97%, 98%, 99% or more of identity with SEQ ID NOs: 4-6, respectively.

In one embodiment of the present antibody or antigen-binding fragment, at least one, preferably at least two, more preferably all three, of its HCDRs 1-3 comprise SEQ ID NOs: 1-3, respectively; and at least one, preferably at least two, more preferably all three, of its LCDRs 1-3 comprise SEQ ID NOs: 4-6, respectively.

In one embodiment, the present antibody or antigen-binding fragment comprises HCDRs 1-3 and LCDRs 1-3 having the sequences of SEQ ID NOs: 1-6, respectively.

In one embodiment of the present antibody or antibody-binding fragment, any of HCDR1, HCDR2 and/or HCDR3 has an amino acid sequence that shares at least 90%, 95%, 96%, 97%, 98%, 99% or more of identity with SEQ ID NOs: 1-3, respectively; and any of LCDR1, LCDR2 and/or LCDR3 has an amino acid sequence that shares at least 90%, 95%, 96%, 97%, 98%, 99% or more of identity with SEQ ID NOs: 4-6, respectively.

### I.2.2 VH and VL Sequences

In one embodiment, the present antibody or antigen-binding fragment has a VH amino acid sequence comprising or consisting of SEQ ID NO: 7.

In one embodiment, the VH amino acid sequence comprises or consists of SEQ ID NO: 7 having 1, 2, 3, 4, 5, 6, 7, 8, 9, 10, 11, 12, 13, 14, 15, 16, 17, 18, 19, 20, or more amino acid modifications (e.g., substitutions).

In one embodiment, the VH amino acid sequence comprises the HCDRs (e.g., SEQ ID NOs: 1-3) described above and shares at least 70%, 75%, 80%, 85%, 90%, 95%, 96%, 97%, 98%, 99% or more identity with SEQ ID NO: 7.

In one embodiment, the present antibody or antigen-binding fragment has a VL amino acid sequence comprising or consisting of SEQ ID NO: 8.

In one embodiment, the VL amino acid sequence comprises or consists of SEQ ID NO: 8 having 1, 2, 3, 4, 5, 6, 7, 8, 9, 10, 11, 12, 13, 14, 15, 16, 17, 18, 19, 20, or more amino acid modifications (e.g., substitutions).

In one embodiment, the VL amino acid sequence comprises the LCDRs (e.g., SEQ ID NOs: 4-6) described above and shares at least 70%, 75%, 80%, 85%, 90%, 95%, 96%, 97%, 98%, 99% or more identity with SEQ ID NO: 8.

In one embodiment, the VH comprises or consists of SEQ ID NO: 7 and/or the VL comprises or consists of SEQ ID NO: 8 with 1, 2, 3, 4, 5, 6, 7, 8, 9, 10, 11, 12, 13, 14, 15, 16, 17, 18, 19, 20, or more amino acid modifications (e.g., substitutions).

In one embodiment, the VH and the VL comprise the CDRs (e.g., SEQ ID NOs: 1-6) as described above and share at least 70%, 75%, 80%, 85%, 90%, 95%, 96%, 97%, 98%, 99% or more identity with SEQ ID NOs: 7 and 8, respectively.

In one embodiment, the amino acid modification may be an insertion, a deletion, or a substitution. In one embodiment, the amino acid modification does not significantly affect the binding characteristics of the antibody or antigen-binding fragment thereof containing the modification. Specified variable region and CDR sequences may comprise 1, 2, 3, 4, 5, 6, 7, 8, 9, 10, 11, 12, 13, 14, 15, 16, 17, 18, 19, 20, or more amino acid insertions, deletions, and/or substitutions.

In one embodiment, the amino acid modification is a substitution made preferably with a conservative amino acid. A conservative amino acid is an amino acid having a side chain with similar physicochemical properties to those of the original amino acid. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine), and aromatic side chains (e.g., tyrosine, phenylalanine, tryptophan, histidine). Thus, one or more amino acid residues within the CDRs and/or variable regions of the present antibody or antigen-binding fragment can be replaced with other amino acid residues from the same side chain family, and the modified antibody or antigen-binding fragment can be tested for retained function (e.g., binding to IL-23R) using the assays described herein. In another embodiment, a string of amino acids within the CDRs and/or variable regions of the present antibody or antigen-binding fragment can be replaced with a structurally similar string that differs in order and/or composition of side chain family members.

In one embodiment, the present antibody or antigen-binding fragment comprises:
a VH comprising at least one (preferably three) HCDR as defined herein, and comprising or consisting of SEQ ID NO: 7 or an amino acid sequence with at least 70%, 75%, 80%, 85%, 90%, 95%, 96%, 97%, 98%, 99% or more identity thereto; and
a VL comprising at least one (preferably three) LCDR as defined herein, and comprising or consisting of SEQ ID NO: 8, or an amino acid sequence with at least 70%, 75%, 80%, 85%, 90%, 95%, 96%, 97%, 98%, 99% or more identity thereto.

In one embodiment, the antibody or antigen-binding fragment thereof comprises a VH comprising or consisting of SEQ ID NO: 7; and a VL comprising or consisting of SEQ ID NO: 8.

### I.2.3 Linkers

In one embodiment, the present antigen-binding fragment comprises a linker that links its VH and VL. In one embodiment, the antigen-binding fragment comprises, from N-terminus to C-terminus, the VL, a linker, and the VH. In another embodiment, the antigen-binding fragment comprises, from N-terminus to C-terminus, the VH, a linker, and the VL.

In one embodiment, the linker is a peptide linker, having a length ranging from, e.g., 2 to 20 or 2 to 15 amino acids.

For example, a glycine-serine doublet provides a particularly suitable linker (GS linker). In one embodiment, the linker is a GS linker. Examples of GS linkers include, but are not limited to, GS linkers, G₂S linkers (e.g., GGS and (GGS)₂), G₃S linkers, and G₄S linkers.

G₃S linkers comprise the amino acid sequence (Gly-Gly-Gly-Ser)ₙ or (GGGS)ₙ (SEQ ID NO: 9), where n is a positive integer equal to or greater than 1 (such as, example, n=1, n=2, n=3, n=4, n=5, n=6, n=7, n=8, n=9, or n=10). Examples of G₃S linkers include, but are not limited to, GGGSGGGSGGGSGGGS (SEQ ID NO: 10).

Examples of G₄S linkers include, but are not limited to, (Gly₄-Ser) corresponding to GGGGS (SEQ ID NO: 11); (Gly₄-Ser)₂ corresponding to GGGGSGGGGS (SEQ ID NO: 12); (Gly₄-Ser)₃ corresponding to GGGGSGGGGSGGGGS (SEQ ID NO: 13); and (Gly₄-Ser)₄ corresponding to GGGGSGGGGSGGGGSGGGGS (SEQ ID NO: 14). In one embodiment, the linker is a (G₄S)₃ linker (SEQ ID NO: 13).

### I2.4 Types of Antibodies and Fragments

In one embodiment, the antibody or antigen-binding fragment thereof according to the present disclosure is a humanized antibody.

In one embodiment, the antibody or antigen-binding fragment thereof according to the present disclosure is an antigen-binding fragment of an antibody, such as, for example, a single chain antibody, a Fv, a Fab, a Fab′, a Fab′-SH, a F(ab)′₂, a Fd, a defucosylated antibody, a diabody, a triabody or a tetrabody.

In one embodiment, the antibody or antigen-binding fragment thereof according to the present disclosure binds to a human IL-23R alpha subunit with high affinity. In an embodiment, the antibody or antigen-binding fragment thereof according to the present disclosure comprises CDRs as defined herein and binds to a human IL-23R alpha subunit with high affinity.

In one embodiment, the antibody or antigen-binding fragment thereof according to the present disclosure binds to a mouse IL-23R alpha subunit with high affinity. In one embodiment, the antibody or antigen-binding fragment thereof according to the present disclosure comprises CDRs as defined herein and binds to a mouse IL-23R alpha subunit with high affinity.

In one embodiment, the antibody or antigen-binding fragment thereof according to the present disclosure is capable of binding to a human IL-23R alpha subunit with an EC50 of less than 200 nM, 100 nM, 90 nM, 80 nM, 70 nM, 60 nM, 50 nM, 40 nM or 30 nM, in particular of less than 100 nM, in particular less than 40 nM. In one embodiment, the antibody or antigen-binding fragment thereof according to the present disclosure is capable of binding to a mouse IL-23R alpha subunit with an EC50 of less than 200 nM, 100 nM, 90 nM, 80 nM, 70 nM, 60 nM, 50 nM, 40 nM or 30 nM, in particular of less than 100 nM, in particular less than 60 nM.

In one embodiment, the antibody or antigen-binding fragment thereof according to the present disclosure is capable of cross-reacting with both human and mouse IL-23R. In an embodiment, the antibody or antigen-binding fragment thereof according to the present disclosure comprises CDRs as defined herein and is capable of cross-reacting with both human and mouse IL-23R.

I.2.5 scFv Full Sequence

In one embodiment, the present antigen-binding fragment is an scFv comprising CDRs as defined herein and comprises or consists of SEQ ID NO: 15 or an amino acid sequence with at least 70%, 75%, 80%, 85%, 90%, 95%, 96%, 97%, 98%, 99%, or more of identity thereto.

In one embodiment, the scFv binds to a human IL-23R alpha subunit with high affinity. In an embodiment, the scFv comprises CDRs as defined herein and binds to a human IL-23R alpha subunit with high affinity. In an embodiment, the scFv comprises or consists of SEQ ID NO: 15 or an amino acid sequence with at least 70%, 75%, 80%, 85%, 90%, 95%, 96%, 97%, 98%, 99%, or more of identity thereto and binds to a human IL-23R alpha subunit with high affinity.

In one embodiment, the scFv binds to a mouse IL-23R alpha subunit with high affinity. In an embodiment, the scFv comprises CDRs as defined herein and binds to a mouse IL-23R alpha subunit with high affinity. In an embodiment, the scFv comprises or consists of SEQ ID NO: 15 or an amino acid sequence with at least 70%, 75%, 80%, 85%, 90%, 95%, 96%, 97%, 98%, 99%, or more of identity thereto and binds to a mouse IL-23R alpha subunit with high affinity.

In one embodiment, the scFv according to the present disclosure is capable of binding to a human IL-23R alpha subunit with an EC50 of less than 200 nM, 100 nM, 90 nM, 80 nM, 70 nM, 60 nM, 50 nM, 40 nM or 30 nM, in particular of less than 100 nM, in particular less than 40 nM. In one embodiment, the scFv according to the present disclosure is capable of binding to a mouse IL-23R alpha subunit with an EC50 of less than 200 nM, 100 nM, 90 nM, 80 nM, 70 nM, 60 nM, 50 nM, 40 nM or 30 nM, in particular of less than 100 nM, in particular less than 60 nM.

In one embodiment, the scFv is capable of cross-reacting with both human and mouse IL-23R. In an embodiment, the scFv comprises CDRs as defined herein and is capable of cross-reacting with both human and mouse IL-23R. In an embodiment, the scFv comprises or consists of SEQ ID NO: 15 or an amino acid sequence with at least 70%, 75%, 80%, 85%, 90%, 95%, 96%, 97%, 98%, 99%, or more of identity thereto and binds to both human and mouse IL-23R.

### I.3 Nucleic Acid

Another objective of the present disclosure is an isolated nucleic acid encoding the present isolated anti-IL-23R antibody or antigen-binding fragment thereof.

An “isolated nucleic acid”, as used herein, is intended to refer to a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. This refers to the nucleic acid as originally isolated and does not exclude genes or sequences later added to the isolated nucleic acid by the hand of man.

In one embodiment, the nucleic acid encodes at least a VH or a VL of the present antibody or antigen-binding fragment. In one embodiment, the nucleic acid encodes variable and constant regions of the heavy or light chain of the present antibody or antigen-binding fragment. In one embodiment, the nucleic acid encodes both the heavy and light chains of the antibody or antigen-binding fragment.

In one embodiment, the nucleic acid herein comprises or consists of a nucleotide sequence encoding the VH of the present antibody or antigen-binding fragment, wherein said nucleotide sequence is SEQ ID NO: 16 or a nucleotide sequence with at least 70%, 75%, 80%, 85%, 90%, 95%, 96%, 97%, 98%, 99%, or more identity thereto.

In one embodiment, the nucleic acid herein comprises or consists of a sequence encoding the VL of the present antibody or antigen-binding fragment, wherein said nucleotide sequence is SEQ ID NO: 17 or a nucleotide sequence with at least 70%, 75%, 80%, 85%, 90%, 95%, 96%, 97%, 98%, 99%, or more identity thereto.

In one embodiment, the nucleic acid herein comprises nucleotide sequences encoding the VH and VL of the present antibody or antigen-binding fragment. In a further embodiment, the nucleic acid herein comprises SEQ ID NOs: 16 and 17.

In one embodiment, the nucleic acid herein further comprises a linker nucleotide sequence between the VL and VH coding sequences. In a further embodiment, the linker nucleotide sequence comprises or consists of SEQ ID NO: 18.

In one embodiment, the nucleic acid herein comprises or consists of SEQ ID NO: 19 or a nucleotide sequence with at least 70%, 75%, 80%, 85%, 90%, 95%, 96%, 97%, 98%, 99%, or more identity thereto.

### I.4. Production of Antibodies and Antigen-Binding Fragments

The present disclosure also provides a vector for expression the present antibody or antigen-binding fragment thereof and a method of using the vector for producing the antibody or antigen-binding fragment.

In general, a suitable vector contains an origin of replication functional in at least one host organism, a promoter sequence, convenient restriction endonuclease sites, one or more selectable markers, and optionally an enhancer.

Examples of promoters and enhancers used in an expression vector for mammalian cells include, but are not limited to, early promoter and enhancer of SV40, LTR promoter, and enhancer of Moloney mouse leukemia virus, and the promoter and enhancer of immunoglobulin H chain. See also Section II below for additional examples of transcriptional regulatory sequences.

Another objective of the present disclosure is a method of producing and purifying the isolated antibody or antigen-binding fragment thereof as described herein. In one embodiment, the method comprises:
introducing in vitro or ex vivo an expression vector comprising an expression cassette for the antibody or antigen-binding fragment into a competent host cell (e.g., mammalian cells such as CHO cells and NS0 cells);
culturing in vitro or ex vivo the transformed host cells under conditions suitable for expression of the antibody or antigen-binding fragment thereof;
optionally selecting the cells which express and/or secrete said antibody or antigen-binding fragment; and
recovering the expressed antibody or antigen-binding fragment from the cell culture, and
optionally purifying the recovered antibody or antigen-binding fragment.

Methods to purify a protein, in particular an antibody or antigen-binding fragment, are well-known in the art and include, without limitation, protein A-Sepharose, gel electrophoresis, and chromatography (e.g., affinity chromatography such as affinity chromatography on protein L agarose).

### II. Fusion Proteins

The present disclosure further relates to a fusion protein comprising the antibody or an antigen-binding fragment thereof of the present disclosure. In one embodiment, said fusion protein is a chimeric antigen receptor (CAR).

### II.1 CAR

An objective of the present disclosure is a CAR specific for at least one IL-23R. The CAR may comprise (i) an extracellular binding domain comprising an anti-IL-23R antigen-binding fragment described herein, (ii) optionally an extracellular hinge domain, (iii) a transmembrane domain, (iv) an intracellular signaling domain, and (v) optionally a tag and/or a leader sequence. In one embodiment, the CAR comprises one or more polypeptides, such as, for example, two polypeptides.

### II.1.1. Extracellular Binding Domain

In one embodiment, the extracellular binding domain of the CAR comprises or consists of an antigen-binding fragment of the present disclosure. In one embodiment, the antigen-binding fragment comprises at least one of HCDR1-3 having SEQ ID NOs: 1-3, respectively; and/or at least one of LCDR1-3 having SEQ ID NOs: 4-6, respectively. In a further embodiment, the antigen-binding fragment in the extracellular binding domain of the CAR comprises HCDR1-3 and LCDR1-3 having the sequences of SEQ ID NOs: 1-6, respectively. In one embodiment, the extracellular binding domain of the CAR comprises an antigen-binding fragment comprising a VH having the sequence of SEQ ID NO: 7, or a sequence having at least about 70%, preferably at least about 75%, 80%, 85%, 90%, 95%, or more identity to SEQ ID NO: 7; and a VL having the sequence of SEQ ID NO: 8, or a sequence having at least about 70%, preferably at least about 75%, 80%, 85%, 90%, 95%, or more identity to SEQ ID NO: 8.

In one embodiment, the extracellular binding domain of the CAR comprises or consists of an anti-IL-23R scFv with a peptide linker between the VH and VL, wherein the peptide linker comprises SEQ ID NO: 13 or a sequence having at least about 90%, 95%, or more identity thereto.

In one embodiment, the extracellular binding domain of the CAR comprises or consists of an anti-IL-23R scFv comprising SEQ ID NO: 15 or a or sequence having at least about 90%, 95%, or more identity thereto.

### II.1.2. Hinge Domain

In one embodiment, the extracellular IL-23R-binding domain is connected to a trans-membrane domain by a hinge domain.

In one embodiment, the hinge domain comprises or consists of about 2 to about 100 amino acids, e.g., from about 2 to about 75 amino acids.

In one embodiment, the hinge domain is a peptide linker having a length ranging from, e.g., 2 to 20 or 2 to 15 amino acids, as described herein.

In one embodiment, the hinge domain comprises or consists of an amino acid sequence derived from a CD8 hinge (e.g., SEQ ID NO: 20) or an amino acid sequence with at least about 95% (e.g., about 96%, 97%, 98%, or 99%) identity to SEQ ID NO: 20. In one embodiment, the hinge domain is a CD8 hinge encoded by SEQ ID NO: 21 or a nucleotide sequence with at least about 95% (e.g., about 96%, 97%, 98%, or 99%) identity to SEQ ID NO: 21.

### I.1.3. Transmembrane Domain

Examples of transmembrane domains that may be used in the present CAR include, but are not limited to, transmembrane domains of an alpha or beta chain of a T cell receptor (TCR); or of CD28, CD3 gamma, CD3 delta, CD3 epsilon, CD3 zeta, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, PD1, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C.

In one embodiment, the transmembrane domain comprises or consists of an amino acid sequence derived from a CD8 transmembrane domain (e.g., SEQ ID NO: 22) or an amino acid sequence with at least about 95% (e.g., about 96%, 97%, 98% or 99%) identity to SEQ ID NO: 22. In one embodiment, the transmembrane domain is a CD8 transmembrane domain encoded by SEQ ID NO: 23 or a nucleotide sequence with at least about 95% (e.g., about 96%, 97%, 98% or 99%) identity to SEQ ID NO: 23.

In another embodiment, the transmembrane domain comprises or consists of an amino acid sequence derived from a CD28 transmembrane domain (e.g., SEQ ID NO: 24) or an amino acid sequence with at least about 95% (e.g., about 96%, 97%, 98% or 99%) identity to SEQ ID NO: 24. In one embodiment, the transmembrane domain is a CD28 transmembrane domain encoded by SEQ ID NO: 25 or a nucleotide sequence with at least about 95% (e.g., about 96%, 97%, 98% or 99%) identity to SEQ ID NO: 25.

In another embodiment, the transmembrane domain comprises or consists of an amino acid sequence derived from a 4-1BB (CD137) transmembrane domain (e.g., SEQ ID NO: 26) or an amino acid sequence with at least about 95% (e.g., about 96%, 97%, 98% or 99%) identity to SEQ ID NO: 26. In one embodiment, the transmembrane domain is a 4-1BB transmembrane domain encoded by SEQ ID NO: 27 or a nucleotide sequence with at least about 95 (e.g., 96%, 97%, 98% or 99%) identity to SEQ ID NO: 27.

In another embodiment, the transmembrane domain comprises or consists of an amino acid sequence derived from a TNFR2 transmembrane domain (e.g., SEQ ID NO: 28) or an amino acid sequence with at least about 95% (e.g., about 96%, 97%, 98% or 99%) identity to SEQ ID NO: 28. In one embodiment, the transmembrane domain is a TNFR2 transmembrane domain encoded by SEQ ID NO: 29 or a nucleotide sequence with at least about 95% (e.g., about 96%, 97%, 98% or 99%) identity to SEQ ID NO: 29.

In one embodiment, the transmembrane domain may be completely artificial and may comprise, for example, predominantly hydrophobic amino acids such as valine and leucine.

### I.1.4. Intracellular Signaling Domain

In one embodiment, the intracellular signaling domain of the present CAR may comprise the entire intracellular portion, or the entire native intracellular signaling domain, of the molecule from which it is derived, or a functional fragment or derivative thereof.

In one embodiment, the intracellular signaling domain comprises a T cell primary signaling domain (or a sequence derived therefrom) and optionally one or more intracellular domains of a T cell costimulatory molecule (or sequence(s) derived therefrom).

In one embodiment, the intracellular signaling domain comprises or consists of a primary signaling domain.

In one embodiment, the intracellular signaling domain comprises or consists of one or more intracellular domains of a T cell costimulatory molecule. In one embodiment, the intracellular signaling domain consists of one or more intracellular domains of a T cell costimulatory molecule.

In another embodiment, the intracellular signaling domain comprises or consists of at least one costimulatory domain and a primary signaling domain.

In another embodiment, the intracellular signaling domain comprises or consists of at least two costimulatory domains and a primary signaling domain.

In one embodiment, the T cell primary signaling domain comprises or consists of a functional signaling domain of CD3 zeta.

In one embodiment, the T cell primary signaling domain comprises or consists of the amino acid sequence of the CD3 zeta intracellular domain of SEQ ID NO: 30, or an amino acid sequence with at least about 95% (e.g., 96%, 97%, 98% or 99%) identity to SEQ ID NO: 30. In one embodiment, the CD3 zeta primary signaling domain comprises or consists of an amino acid sequence having at least one, two, or three modifications—but not more than 20, 10 or 5 modifications—of SEQ ID NO: 30.

In one embodiment, the CD3 zeta primary signaling domain is encoded by SEQ ID NO: 31 or a nucleotide sequence with at least about 95 (e.g., about 96%, 97%, 98% or 99%) identity to SEQ ID NO: 31.

T cell primary signaling domains that act in a stimulatory manner may comprise signaling motifs known as immunoreceptor tyrosine-based activation motifs (ITAMS). In one embodiment, the T cell primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four, or more ITAM motifs.

In one embodiment, the intracellular signaling domain of the present CAR comprises a T cell primary signaling domain (e.g., a CD3 zeta signaling domain) combined with one or more costimulatory signaling domains.

The costimulatory signaling domains may be derived from the intracellular domains of T cell costimulatory molecules or other cell surface molecules expressed on immune cells. Examples of costimulatory signal domains may be those derived from the intracellular domains of CD28, CD27, 4-1BB (CD137), an MHC class I molecule, BTLA, a Toll ligand receptor, OX40, CD30, CD40, PD-1, ICOS (CD278), lymphocyte function-associated antigen-1 (LFA-1), CD2, CD7, LIGHT, NKG2C, B7-H3, a ligand that specifically binds with CD83, CDS, ICAM-1, GITR, ARHR, BAFFR, HVEM (LIGHTR), SLAMF7, NKp80 (KLRF1), NKp44, NKp30, NKp46, CD160 (BY55), CD19, CD19a, CD4, CD8alpha, CD8beta, IL2ra, IL6Ra, IL2Rbeta, IL2R gamma, IL7R alpha, IL-13RA1/RA2, IL-33R(IL1RL1), IL-IORA/RB, IL-4R, IL-5R (CSF2RB), IL-21R, ITGA4, VLA1, CD49a, ITGA4, IA4, CD49D, ITGA6, VLA-6, CD49f, ITGAD, CD11d, ITGAE, CD103, ITGAL, CD11a/CD18, ITGAM, CD11b, ITGAX, CD11c, ITGB1, CD29, ITGB2, CD18, ITGB7, NKG2D, NKG2C, CTLA-4 (CD152), CD95, TNFR1 (CD120a/TNFRSF1A), TNFR2 (CD120b/TNFRSF1B), TGFbR1/2/3, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, common gamma chain, a ligand that specifically binds with CD83, NKp44, NKp30, NKp46, NKG2D, and any combination thereof.

In one embodiment of the present disclosure, the present CAR comprises at least one intracellular domain of a T cell costimulatory molecule selected from the group comprising CD28, TNFR2, 4-1BB, ICOS, CD27, OX40, CTLA4, and PD-1.

In one embodiment, the T cell costimulatory signaling domain comprises or consists of an amino acid sequence derived from a CD28 intracellular domain (e.g., SEQ ID NO: 32) or an amino acid sequence with at least about 95% (e.g., about 96%, 97%, 98% or 99%) identity to SEQ ID NO: 32. In one embodiment, the T cell costimulatory signaling domain comprises or consists of an amino acid sequence having at least one, two or three modifications—but not more than 20, 10 or 5 modifications—of an amino acid sequence of SEQ ID NO: 32. In one embodiment, the T cell costimulatory signaling domain is encoded by SEQ ID NO: 33 or a nucleotide sequence with at least about 95% (e.g., about 96%, 97%, 98% or 99%) identity to SEQ ID NO: 33.

In one embodiment, the T cell costimulatory signaling domain comprises or consists of an amino acid sequence derived from a 4-1BB intracellular domain (e.g., SEQ ID NO: 34) or an amino acid sequence with at least about 95% (e.g., about 96%, 97%, 98% or 99%) identity to SEQ ID NO: 34. In one embodiment, the T cell costimulatory signaling domain comprises or consists of an amino acid sequence having at least one, two or three modifications—but not more than 20, 10 or 5 modifications—of an amino acid sequence of SEQ ID NO: 34. In one embodiment, the T cell costimulatory signaling domain is encoded by SEQ ID NO: 35 or a nucleotide sequence with at least about 95% (e.g., about 96%, 97%, 98% or 99%) identity to SEQ ID NO: 35.

In one embodiment, the T cell costimulatory signaling domain comprises or consists of an amino acid sequence derived from a CD27 intracellular domain (e.g., SEQ ID NO: 36) or an amino acid sequence with at least about 95% (e.g., about 96%, 97%, 98% or 99%) identity to SEQ ID NO: 36. In one embodiment, the T cell costimulatory signaling domain comprises or consists of an amino acid sequence having at least one, two or three modifications—but not more than 20, 10 or 5 modifications—of an amino acid sequence of SEQ ID NO: 36. In one embodiment, the T cell costimulatory signaling domain is encoded by SEQ ID NO: 37 or a nucleotide sequence with at least about 95% (e.g., about 96%, 97%, 98% or 99%) identity to SEQ ID NO: 37.

In one embodiment, the T cell costimulatory signaling domain comprises or consists of an amino acid sequence derived from a TNFR2 intracellular domain (e.g., SEQ ID NO: 38) or an amino acid sequence with at least about 95% (e.g., about 96%, 97%, 98% or 99%) identity to SEQ ID NO: 38. In one embodiment, the T cell costimulatory signaling domain comprises or consists of an amino acid sequence having at least one, two or three modifications—but not more than 20, 10 or 5 modifications—of an amino acid sequence of SEQ ID NO: 38. In one embodiment, the T cell costimulatory signaling domain is encoded by SEQ ID NO: 39 or a nucleotide sequence with at least about 95% (e.g., about 96%, 97%, 98% or 99%) identity to SEQ ID NO: 39.

In one embodiment, the intracellular signaling domain of the present CAR comprises:
the amino acid sequence of a CD28 intracellular domain of SEQ ID NO: 32 or an amino acid sequence with at least about 95% (e.g., about 96%, 97%, 98% or 99%) identity to SEQ ID NO: 32; and
the amino acid sequence of a CD3 zeta intracellular domain of SEQ ID NO: 30 or an amino acid sequence with at least about 95% (e.g., about 96%, 97%, 98% or 99%) identity to SEQ ID NO: 30.

In one embodiment, the intracellular signaling domain of the present CAR comprises at least two different domains (e.g., a primary signaling domain and at least one intracellular domain of a T cell costimulatory molecule) that may be linked to each other in a random order or in a specified order.

Optionally, a peptide linker may be used to connect distinct signaling domains. In one embodiment, a glycine-serine doublet (GS) is used as a suitable linker. In one embodiment, a single amino acid, e.g., an alanine (A) or a glycine (G), is used as a linker. Other examples of peptide linkers are described in Section I above.

In one embodiment, the intracellular signaling domain of the present CAR comprises two or more (e.g., 2, 3, 4, 5, or more) costimulatory signaling domains. In one embodiment, the two or more costimulatory signaling domains are separated by a peptide linker such as those described herein.

In one embodiment, the intracellular signaling domain of the present CAR comprises the primary signaling domain of CD3 zeta (e.g., SEQ ID NO: 30) and the co-stimulatory signaling domain of CD28 (e.g., SEQ ID NO: 32).

### I.1.5. Leader Sequence

In one embodiment, the CAR of the present disclosure further comprises a leader sequence located N-terminal to the IL-23R-specific extracellular binding domain. A leader sequence may allow cell surface expression of the CAR protein after the protein is secreted from the Golgi complex.

Suitable leader sequence of the present CAR includes a CD8 leader sequence, a CD25 leader sequence, or a Igk leader sequence.

A non-limiting example of leader sequence is a leader sequence of CD8 that may comprise or consists of SEQ ID NO: 40. In one embodiment, the leader sequence comprises or consists of an amino acid sequence derived from a CD8 leader sequence (e.g., SEQ ID NO: 40) or an amino acid sequence with at least about 95% (e.g., about 96%, 97%, 98%, or 99%) identity to SEQ ID NO: 40. Preferably, the leader sequence consists of SEQ ID NO: 40.

In one embodiment, the nucleotide sequence encoding the leader sequence comprises or consists of a nucleotide sequence coding for a CD8 leader sequence (e.g., SEQ ID NO: 41) or a nucleotide sequence with at least about 95% (e.g., about 96%, 97%, 98% or 99%) identity to SEQ ID NO: 41. Preferably, the leader sequence is encoded by a sequence consisting of SEQ ID NO: 41.

A further non-limiting example of leader sequence is a leader sequence of CD25 that may comprise or consists of SEQ ID NO: 58. In one embodiment, the leader sequence comprises or consists of an amino acid sequence derived from a CD25 leader sequence (e.g., SEQ ID NO: 58) or an amino acid sequence with at least about 95% (e.g., about 96%, 97%, 98%, or 99%) identity to SEQ ID NO: 58. Preferably, the leader sequence consists of SEQ ID NO: 58.

In one embodiment, the nucleotide sequence encoding the leader sequence comprises or consists of a nucleotide sequence coding for a CD25 leader sequence (e.g., SEQ ID NO: 59) or a nucleotide sequence with at least about 95% (e.g., about 96%, 97%, 98% or 99%) identity to SEQ ID NO: 59. Preferably, the leader sequence is encoded by a sequence consisting of SEQ ID NO: 59.

### II.1.6. Tag

In one embodiment, the CAR further comprises a tag for, e.g., quality control, enrichment, and tracking in vivo. Said a tag may be localized at the N-terminus or the C-terminus of the CAR, or internally within the CAR polypeptide. Examples of tags include, but are not limited to, Hemagglutinin Tag, Poly Arginine Tag, Poly Histidine Tag, Myc Tag, Strep Tag, S-Tag, HAT Tag, 3× Flag Tag, Calmodulin-binding peptide Tag, SBP Tag, Chitin binding domain Tag, GST Tag, Maltose-Binding protein Tag, Fluorescent Protein Tag, T7 Tag, V5 Tag, and Xpress Tag.

In one embodiment, the CAR of the present disclosure comprises a HA tag (SEQ ID NO: 42). In one embodiment the tag is encoded by SEQ ID NO: 43 or a nucleotide sequence with at least about 95% (e.g., about 96%, 97%, 98% or 99%) identity to SEQ ID NO: 43.

### II.1.7. Exemplary CAR

According to one embodiment, the CAR of the present disclosure comprises an IL-23R-binding domain (e.g., a domain comprising or consisting of SEQ ID NO: 15), optionally an extracellular hinge domain, a transmembrane domain, a single intracellular domain of a T cell costimulatory molecule and a T cell primary signaling domain. Preferably, the CAR of the present disclosure further comprises a leader sequence.

In one embodiment, the CAR of the present disclosure comprises an IL-23R-binding domain (e.g., SEQ ID NO: 15); a transmembrane domain of CD8 (e.g., SEQ ID NO: 22); an intracellular domain of CD28 (e.g., SEQ ID NO: 32); and a CD3 zeta primary signaling domain (e.g., SEQ ID NO: 30). In one embodiment, the CAR of the present disclosure further comprises a leader sequence.

In one embodiment, the CAR of the present disclosure comprises an IL-23R-binding domain (e.g., SEQ ID NO: 15); a hinge domain of CD8 (e.g., SEQ ID NO: 20); a transmembrane domain of CD8 (e.g., SEQ ID NO: 22); an intracellular domain of CD28 (e.g., SEQ ID NO: 32); and a CD3 zeta primary signaling domain (e.g., SEQ ID NO: 30).

In one embodiment, the CAR of the present disclosure comprises an IL-23R-binding domain (e.g., SEQ ID NO: 15); a hinge domain of CD8 (e.g., SEQ ID NO: 20); a transmembrane domain of CD8 (e.g., SEQ ID NO: 22); an intracellular domain of CD28 (e.g., SEQ ID NO: 32); a CD3 zeta primary signaling domain (e.g., SEQ ID NO: 30), and a leader sequence. In one embodiment the leader sequence comprises or consists of SEQ ID NO: 40, SEQ ID NO: 58, SEQ ID NO: 60 or SEQ ID NO: 62. Preferably, the leader sequence comprises or consists of SEQ ID NO: 40 or SEQ ID NO: 58. More preferably, the leader sequence comprises or consists of SEQ ID NO: 58.

In one embodiment, the CAR of the present disclosure comprises an anti-IL-23R scFv (e.g., an scFv comprising or consisting of SEQ ID NO: 15), a hinge region of CD8, a transmembrane domain of human CD8, an intracellular domain of human CD28 and an intracellular domain of human CD3ζ. In one embodiment, the CAR of the present disclosure further comprises a leader sequence.

In one embodiment, said CAR comprises or consists of SEQ ID NO: 44 or an amino acid sequence with at least about 95% (e.g., about 96%, 97%, 98% or 99%) identity to SEQ ID NO: 44.

The CAR of the present disclosure is a new and cross-reactive CAR, which is able to bind mouse and human IL-23R (Example 3 and FIG. 14). In these exemplary results, a CAR of the present disclosure exhibit a similar binding profile between the human and the mouse homologue of IL23R. In these exemplary results, CAR #2 of the present disclosure demonstrates about a 1.4-fold difference in binding affinity between human IL23R and mouse IL23R, whereas CAR #1 demonstrates about a 3-fold difference in binding affinity between human IL23R and mouse IL23R. These exemplary results show that CAR #2 of the present disclosure binds to human and mouse IL23R with a more similar affinity as compared to CAR #1. This similarity indicates that the use of CAR #2 in a mouse preclinical model may be more representative of its interaction with human IL23R.

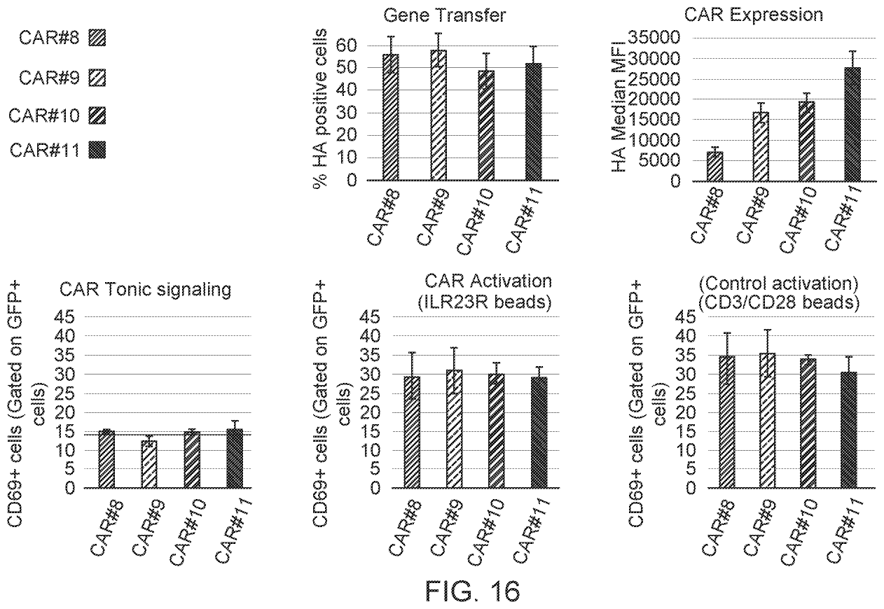

Advantageously, the CAR of the present disclosure showed stable signaling of the antigen-binding portion and a low background activation (Example 4 and FIG. 16).

### II.1.8. Mouse CARs

The present disclosure also provides mouse CARs that comprise an IL-23R-binding domain (e.g., a domain comprising or consisting of SEQ ID NO: 15), optionally an extracellular hinge domain, a transmembrane domain, a single intracellular domain of a T cell costimulatory molecule and a T cell primary signaling domain.

In one embodiment, the mouse CAR of the present disclosure comprises an IL-23R-binding domain (e.g., SEQ ID NO: 15); a transmembrane domain of mouse CD8 (e.g., SEQ ID NO: 50) or mouse CD28 (SEQ ID NO: 52); an intracellular domain of mouse CD28 (e.g., SEQ ID NO: 54); and a mouse CD3 zeta primary signaling domain (e.g., SEQ ID NO: 56). In certain embodiments, the mouse CAR may also comprise a hinge domain of mouse CD8 (e.g., SEQ ID NO: 46) or mouse CD28 (e.g., SEQ ID NO: 48). A mouse CAR with any combination of the above domains is contemplated.

### II.2. Nucleic Acid Encoding a CAR

The present disclosure also relates to a nucleic acid sequence encoding a CAR as described herein. An example of such a nucleic acid sequence is SEQ ID NO: 45 or a degenerate or codon-optimized version thereof.

### II.3. Vector for Expressing a CAR

Another objective of the present disclosure is an expression vector comprising a nucleic acid encoding the CAR herein.

In one embodiment, the nucleic acid encoding the CAR is a DNA. In one embodiment, the nucleic acid encoding the CAR is an RNA. Examples of vectors that may be used in the present disclosure include, but are not limited to, a DNA vector, an RNA vector, a plasmid, an episome, a viral vector (e.g., an animal virus).

In one embodiment, the expression vector may comprise regulatory elements, such as a promoter, an enhancer, and a transcription terminator, to cause or direct expression of the transgene (e.g., CAR) thereon in host cells. The vector may also comprise one or more selectable markers.

Examples of promoters and enhancers used in the expression vector for animal cell include, but are not limited to, early promoter and enhancer of SV40, LTR promoter and enhancer of Moloney mouse leukemia virus, promoter, and enhancer of immunoglobulin H chain and the like. Other examples of suitable constitutive promoters include, but are not limited to, the immediate early cytomegalovirus (CMV) promoter sequence, elongation factor 1α (EF-1α) promoter, phosphoglycerate kinase (PGK) promoter, FOXP3 derived promoter, simian virus 40 (SV40) early promoter, mouse mammary tumor virus (MMTV) promoter, human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter.

Examples of suitable inducible promoters include, but are not limited to, a metallothionine promoter, a glucocorticoid promoter, a progesterone promoter, a cumate promoter and a tetracycline promoter.

Examples of suitable bi-directional promoters include, but are not limited to, the promoters described by Luigi Naldini U.S. Pat. No. 8,501,464, incorporated herein by reference, disclosing a bi-directional promoter comprising i) a first minimal promoter sequence derived from cytomegalovirus (CMV) or mouse mammary tumor virus (MMTV) genomes and ii) a full efficient promoter sequence derived from an animal gene.

Examples of suitable vectors include, but are not limited to, pAGE107, pAGE103, pHSG274, pKCR, pSG1 beta d2-4, and the like.

Examples of plasmids include, but are not limited to, replicating plasmids comprising an origin of replication, or integrative plasmids, such as pUC, pcDNA, pBR, and the like.

A number of viral-based systems have been developed for gene transfer into mammalian cells. Examples of viral vectors include, but are not limited to adenoviral vectors, retroviral vectors, lentiviral vectors, herpes virus vectors and adeno-associated viral (AAV) vectors.

Retroviruses may provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art.

In some embodiments, adenovirus vectors are used. A number of adenovirus vectors are known in the art.

In one embodiment, lentivirus vectors are used.

In one embodiment, AAV vectors are used. As used herein, the term “AAV” covers all serotypes and variants, both naturally occurring and engineered forms. For example, the term encompasses AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8), and AAV type 9 (AAV-9). In one embodiment, the vector is an AAV6 vector. In one embodiment, the AAV is a pseudotype AAV, such as an AAV having an AAV6 capsid and a recombinant genome derived from another AAV serotype (e.g., having ITRs from AAV2).

The recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, 293T cells etc. Detailed protocols for producing such replication-defective recombinant viruses may be found in the art. Insect cells may also be used to produce recombinant viruses such as recombinant AAV.

### I.4. Cell Expressing a CAR

The present disclosure further relates to an immune cell and to an immune cell population engineered to express on the cell surface a CAR as described herein.

In one embodiment, the immune cell is a T cell, such as, a regulatory T cell (Treg), a CD8⁺ T cell, a CD4⁺ T cell, or a NK T cell.

In one embodiment, the immune cell is a tumor-infiltrating lymphocyte (TIL).

The present disclosure also relates to an isolated and/or substantially purified immune cell population, preferably a T cell population, comprising or consisting of immune cells engineered to express on the cell surface a CAR as described herein.

In one embodiment, the immune cells, preferably the T cells, are suppressive for cells expressing on their surface the IL-23R recognized by the CAR.

In one embodiment, the immune cells, preferably the T cells, are cytotoxic for cells expressing on their surface the IL-23R recognized by the CAR.

In one embodiment, the immune cell population, preferably T cell population, comprises or consists of Treg cells, CD8⁺ T cells, CD4⁺ T cells, and/or NK T cells.

In one embodiment, the T cells of the present disclosure are Treg cells.

In one embodiment, the Treg cells in a cell population of the present disclosure all express a CAR described herein and may thus be defined as CAR-monospecific (i.e., all the Treg cells recognize the same antigen (IL-23R)). In one embodiment, the Treg cell population is TCR-monospecific (i.e., all the Treg cells recognize the same antigen with their TCR). In another embodiment, the Treg cell population is TCR-polyspecific (i.e., the Treg cells may recognize different antigens with their TCRs).

In one embodiment, the CAR of the present disclosure, when expressed by a T (e.g., Treg) cell, confers to the T cell the ability to bind to cells expressing IL-23R on their cell surface and to be activated by binding to the IL-23R.

Examples of cells expressing IL-23R include, but are not limited to, Th17 cells, up T cells, neutrophils, γδ T cells, NK cells, NK T cells, dendritic cells, and macrophages.

The immune cell population of the present disclosure (e.g., the T (e.g., Treg) cell population of the present disclosure) may thus be defined as a redirected immune cell population. As used herein, the term “redirected” refers to an immune cell carrying a CAR as described herein, which confers to the immune cell the ability to bind to and be activated by a ligand that is different from the one the immune cell is or would have been specific or be activated by.

In one embodiment, Treg cells of the present disclosure are not cytotoxic. In another embodiment, Treg cells of the present disclosure are cytotoxic.

In one embodiment, Treg cells of the present disclosure may be selected from the group comprising CD4⁺CD25⁺FOXP3⁺ Treg cells, Tr1 cells, TGF-β-secreting Th3 cells, regulatory NK T cells, regulatory γδ T cells, regulatory CD8⁺ T cells, and double negative regulatory T cells.

In one embodiment, the immune cell is a CD4⁺ Treg cell. In one embodiment, the Treg is a thymus-derived Treg or an adaptive or induced Treg. In one embodiment, the Treg cell is a CD4⁺ FOXP3⁺ Treg cell, or a CD4⁺FOXP3⁻ regulatory T cell (Tr1 cell).

In one embodiment, the immune cell is a CD8⁺ Treg cell. In one embodiment, the CD8⁺ Treg cell is selected from the group consisting of a CD8+CD28⁻ Treg cell, a CD8⁺CD103⁺ Treg cell, a CD8⁺FOXP3⁺ Treg cell, a CD8⁺CD122⁺ Treg cell, and any combination thereof. In one embodiment, the regulatory cell is an INFγ⁺IL10⁺IL34⁺CD8⁺CD45RC^{low} Treg cell.

In one embodiment, the immune cells of the present disclosure are human Treg cells.

In one embodiment, the immune cells (e.g., the T or Treg cells) are derived from stem cells, such as induced pluripotent stem cells (iPSC).

As used herein, the term “induced pluripotent stem cells” or “iPSC” refers to pluripotent stem cells derived from non-pluripotent cells (e.g., adult somatic cells) by de-differentiation or reprogramming. In particular, iPSCs may be obtained by introducing a specific set of pluripotency-associated genes (reprogramming factors) into a cell. Reprogramming factors may be, for example, the transcription factors Oct4 (Pou5f1), Sox2, c-Myc, and Klf4.

In one embodiment, the Treg cell has the following phenotype: CD4⁺CD25+, such as CD4⁺CD25⁺CD127⁻ and CD4⁺CD25⁺CD127⁻CD45RA⁺. In one embodiment, the Treg cell has the following phenotype: CD4⁺CD25⁺, such as CD4⁺CD25⁺CD127^{low} and CD4⁺CD25⁺CD127^{low}CD45RA⁺. In one embodiment, the Treg cell has the following phenotype: CD4⁺CD25⁺, such as CD4⁺CD25⁺CD127^{low/-} and CD4⁺CD25⁺CD127^{low/-}CD45RA⁺. In one embodiment, the Treg cell has the following phenotype: FOXP3⁺CD4⁺CD25⁺, such as FOXP3⁺CD4⁺CD25⁺CD127⁻ and FOXP3⁺CD4⁺CD25⁺CD127⁻ CD45RA⁺. In one embodiment, the Treg cell has the following phenotype: FOXP3⁺CD4⁺CD25⁺, such as FOXP3⁺CD4⁺CD25⁺CD127^{low} and FOXP3⁺CD4⁺CD25⁺CD127^{low}CD45RA⁺. In one embodiment, the Treg cell has the following phenotype: FOXP3⁺CD4⁺CD25⁺, such as FOXP3⁺CD4⁺CD25⁺CD127^{low/-} and FOXP3⁺CD4⁺CD25⁺CD127^{low/-}CD45RA⁺.

In one embodiment, the Treg cell has the following phenotype: CD4⁺CD25^{high} such as CD4⁺CD25^{high}CD127⁻ and CD4⁺CD25^{high}CD127⁻CD45RA⁺. In one embodiment, the Treg cell has the following phenotype: CD4⁺CD25^{high} such as CD4⁺CD25^{high}CD127^{low} and CD4⁺CD25^{high}CD127^{low}CD45RA⁺. In one embodiment, the Treg cell has the following phenotype: CD4⁺CD25^{high}, such as CD4⁺CD25highCD127^{low/-} and CD4⁺CD25highCD127^{low/-}CD45RA⁺. In one embodiment, the Treg cell has the following phenotype: FOXP3⁺CD4⁺CD25^{high} such as FOXP3⁺CD4⁺CD25^{high}CD127⁻ and FOXP3⁺CD4⁺CD25^{high}CD127⁻CD45RA⁺. In one embodiment, the Treg cell has the following phenotype: FOXP3⁺CD4⁺CD25^{high} such as FOXP3⁺CD4⁺CD25^{high}CD127^{low} and FOXP3⁺CD4⁺CD25^{high}CD127^{low}CD45RA⁺. In one embodiment, the Treg cell has the following phenotype: FOXP3⁺CD4⁺CD25^{high} such as FOXP3⁺CD4⁺CD25highCD127^{low/-} and FOXP3⁺CD4⁺CD25highCD127^{low/-}CD45RA⁺.

In one embodiment, the Treg cell has a stable FOXP3 expression which is associated with a selective demethylation and/or hypomethylation at a conserved region, the Treg-specific demethylated region (TSDR), within intron 1 of FOXP3 locus.

The expression level of molecules may be determined by flow cytometry, immunofluorescence, or image analysis. To detect intracellular proteins, cells may be fixed and permeabilized prior to flow cytometry analysis.

In one embodiment, the expression level of a molecule in a cell population is indicated by the percentage of cells of the cell population expressing the molecule (i.e., cells “+” for the molecule). The percentage of cells expressing the molecule may be measured by FACS. The expression level of the cell marker of interest may be determined by comparing the Median Fluorescence Intensity (MFI) of the cells from the cell population stained with fluorescently labeled antibody specific for this marker to the fluorescence intensity (FI) of the cells from the same cell population stained with fluorescently labeled antibody with an irrelevant specificity but with the same isotype, the same fluorescent probe and originated from the same specie (referred as isotype control). The cells from the population stained with fluorescently labeled antibody specific for this marker and that show equivalent MFI or a lower MFI than the cells stained with the isotype controls are not expressing this marker and then are designated (−) or negative. The cells from the population stained with fluorescently labeled antibody specific for this marker and that show a MFI value superior to the cells stained with the isotype controls are expressing this marker and then are designated (+) or positive.

The terms “expressing” (i.e., “positive” or “+”) and “not expressing” (i.e., “negative” or “−”) refer to the expression level of the cell marker of interest, in that the expression level of the cell marker corresponding to “+” is high or intermediate, also referred as “+/−,” and the expression level of the cell marker corresponding to “−” is null. The term “low” or “lo” or “lo/−” or “low/−” refers to the expression level of the cell marker of interest, in that the expression level of the cell marker is low by comparison with the expression level of that cell marker in the population of cells being analyzed as a whole. More particularly, the term “lo” refers to a distinct population of cells that express the cell marker at a lower level than one or more other distinct population of cells. The term “high” or “hi” or “bright” refers to the expression level of the cell marker of interest, in that the expression level of the cell marker is high by comparison with the expression level of that cell marker in the population of cells being analyzed as a whole. Generally, cells in the top 2, 3, 4, or 5% of staining intensity are designated “hi,” with those falling in the top half of the population categorized as being “+.” Those cells falling below 50%, of fluorescence intensity are designated as “lo” cells and below 5% as “−” cells.

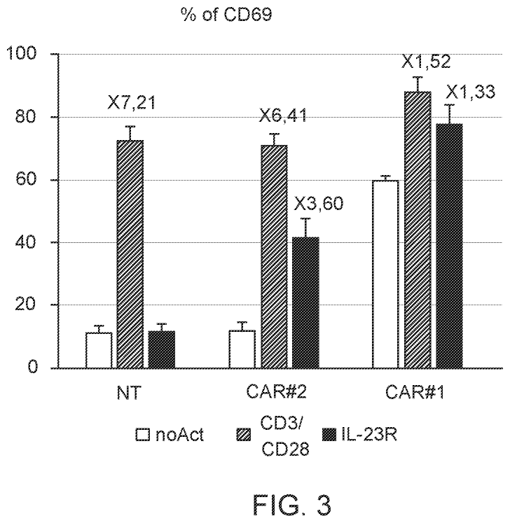

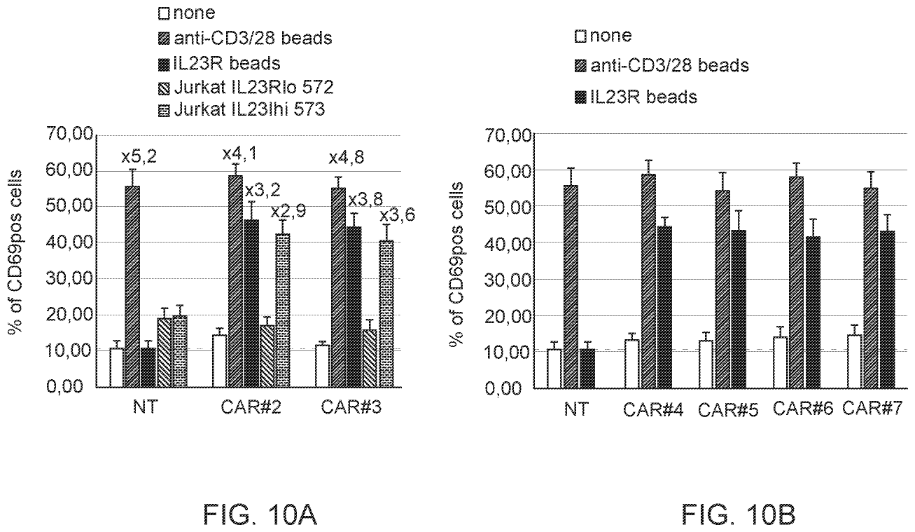

Advantageously, in addition to demonstrating good activation (high signal to background ratio) and good suppressive activity, the immune cells expressing the CAR of the of the present disclosure have been found to have a low tonic signaling (FIG. 3 and FIG. 10).

The term “tonic signaling” as used herein refers to an antigen-independent background of activation. Methods for measuring tonic signaling are well known to the person skilled in the art, and include, without limitation, measuring metabolic activity of the CAR-expressing cells, measuring one or more indicators of cell activation in the absence of stimulation by an antigen recognized by the receptor, measuring one or more phenotypical changes related to cell aging or cell senescence, determining cell cycle progression in the absence of antigenic stimulation; and measuring the size of cells expressing the receptor compared to the size of unmodified cells.

The monitoring of CD69 spontaneous expression by CAR Treg cells as compared to untransduced Treg cells allows determination of tonic signaling intensity.

As demonstrated herein, engineered T cells and engineered Treg cells expressing said CAR constructs of the invention present a low tonic signaling and following CAR engagement, the engineered Treg cells showed highly efficient suppressive activity on T effector cell proliferation, thereby demonstrating the advantage of these Treg cells for cell therapy.

In one embodiment, the CAR of the present disclosure, when expressed by a Treg cell, allows for a reduction of the activation background of said Treg cells as compared to other CAR constructs directed to IL-23R.

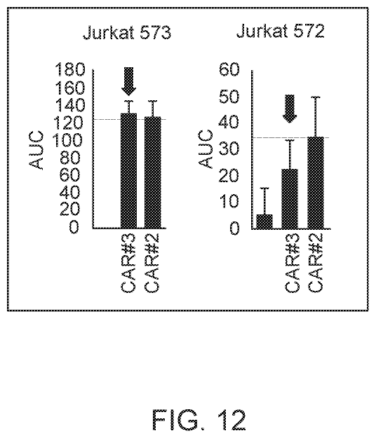

In addition, the immune cells expressing the CAR of the of the present disclosure have been found to have a stronger suppressive activity when the CAR expressing cells are engaged with high levels of expression of IL-23R compared to when engaged with low levels of expression of IL-23R (FIG. 12). Thus, it is expected that the CAR of the present disclosure will reduce off target activity.

In one embodiment, the CAR of the present disclosure, when expressed by a Treg cell, allows for a reduction of off target activity of said Treg cells as compared to other CAR constructs directed to IL-23R.

### III. Composition, Pharmaceutical Composition, Medicament

Another objective of the present disclosure is a composition comprising, consisting essentially of, or consisting of at least one antibody binding to IL-23R as described herein or at least one antigen-binding fragment of said antibody, or at least one nucleic acid or vector encoding an antibody or an antigen-binding fragment of said antibody according to the present disclosure.

Another objective of the present disclosure is a composition comprising, consisting essentially of, or consisting of at least one immune cell or at least one immune cell population comprising the CAR according to the present disclosure.

In one embodiment, said composition is a pharmaceutical composition and further comprises at least one pharmaceutically acceptable excipient.

Consequently, another objective of the present disclosure is a pharmaceutical composition comprising, consisting essentially of, or consisting of at least one immune cell or at least one immune cell population comprising the CAR according to the present disclosure, and at least one pharmaceutically acceptable excipient.

As used herein, “consisting essentially of,” with reference to a composition, means that the at least one antibody or antigen-binding fragment thereof, nucleic acid or expression vector, or the at least one immune cell or immune cell population is the only one therapeutic agent or agent with a biologic activity within said composition.

The term “pharmaceutically acceptable excipient” refers to solvents, dispersion media, coatings, antibacterial and antifungal agents, buffering agents, isotonic agents, stabilizing agents, preservatives, absorption-delaying agents, and the like. Said excipient does not produce an adverse, allergic, or other untoward reaction when administered to a subject, such as a human.

Examples of pharmaceutically acceptable excipients that may be used in the compositions of the present disclosure include, but are not limited to, ion exchangers, alumina, aluminum stearate, lecithin, serum proteins (e.g., human serum albumin), buffer agents (e.g., phosphates), glycine, sorbic acid, potassium sorbate, partial glyceride mixtures of saturated vegetable fatty acids, water, salts or electrolytes (e.g. sodium chloride, protamine sulfate, disodium hydrogen phosphate, potassium hydrogen phosphate, and zinc salts), and polyethylene glycol.

In one embodiment, the pharmaceutical compositions according to the present disclosure comprise vehicles which are pharmaceutically suitable for injection. These may be, for example, isotonic, sterile saline solutions (comprising, e.g., monosodium or disodium phosphate; sodium, potassium, calcium, or magnesium chloride; or mixtures of such salts); or dry (e.g., freeze-dried) compositions which, upon addition of a suitable carrier such as sterilized water or physiological saline, permit the constitution of injectable solutions.

Another objective of the present disclosure is a medicament comprising, consisting essentially of, or consisting of at least one antibody binding to IL-23R as described herein or at least one antigen-binding fragment of said antibody, or at least one nucleic acid or vector encoding an antibody or an antigen-binding fragment of said antibody according to the present disclosure.

Another objective of the present disclosure is a medicament comprising, consisting essentially of, or consisting of a population of immune cells expressing a CAR of present disclosure.

### IV. Administration Route

In one embodiment, the composition, pharmaceutical composition, or medicament according to the present disclosure is administered parenterally, by inhalation spray, rectally, nasally, or via an implanted reservoir.

In one embodiment, the composition, pharmaceutical composition, or medicament is administered by injection, including, without limitation, subcutaneous, intravenous, intramuscular, intra-articular, intra-synovial, intra-sternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

### V. Dosage

In one embodiment, the isolated antibody or antigen-binding fragment thereof, the nucleic acid, expression vector, immune cell or immune cell population, composition, pharmaceutical composition, or medicament according to the present disclosure is to be administered to the subject in need thereof in a therapeutically effective amount.

It will be however understood that the therapeutically effective amount and dosing frequency will be decided by the attending physician within the scope of sound medical judgment. The specific therapeutically effective dose level for any particular patient will depend upon a variety of factors including the disease being treated and the severity of the disease; activity of the isolated antibody or antigen-binding fragment thereof, nucleic acid, expression vector, or immune cell employed; the age, body weight, general health, sex and diet of the subject; the time of administration, route of administration, and rate of excretion of the specific therapeutic agent employed; the duration of the treatment; drugs used in combination or coincidental with the specific therapeutic agent employed; and like factors well known in the medical arts. For example, it is well within the skill of the art to start doses of the compound at levels lower than those required to achieve the desired therapeutic effect and to gradually increase the dosage until the desired effect is achieved. The total dose required for each treatment may be administered by multiple doses or in a single dose.

In one embodiment, the subject (e.g., human) receives a single administration of the immune cell or immune cell population of the present disclosure.

In one embodiment, the subject (e.g., human) receives at least two administrations of the immune cell or immune cell population of the present disclosure.

In one embodiment, the immune cell population is administered once a week, once a month, or once a year to the subject.

In one embodiment, the number of immune cells administered to the subject ranges from about 10² to about 10⁹, from about 10³ to about 10⁸, from about 10⁴ to about 10⁷, or from about 10⁵ to about 10⁶.

In one embodiment, the immune cells are administered to the subject in need thereof in combination with at least one other active agent. In one embodiment, said active agent is an agent that may be used for treating an IL-23R-related disease or disorder. Examples of other active agents include, but are not limited to, glucocorticoids (including, without limitation, dexamethasone, prednisone, prednisolone, methylprednisolone, betamethasone, bedomethasone, tixocortol, triamcinolone, hydrocortisone, budesonide or fludrocortisone), antibodies or antagonists of human cytokines or growth factors (such as, for example, anti-TNF such as infliximab, adalimumab, certolizumab, etanercept; anti-IL1, anti-IL-6, anti-IL-12, anti-IL-17 and anti-IL-23 (e.g. brazikumab, guselkumab, mirikizumab, risankizumab), anti-IL-12/IL-23 (e.g., ustekinumab); or IL-1 receptor antagonist analogs (e.g., anakinra), antibodies to cell surface molecules (e.g., anti-α4 integrin (e.g., natalizumab) anti-integrin β7 (e;g., etrolizumab), anti α4-β7 integrin (e.g., vedolizumab), anti-CD2, anti-CD3 (e.g., visiluzumab), anti-CCR9, anti-LFA1 or anti-ICAM1); JAK inhibitors (e.g., filgotinib, upadacitinib), S1PR modulators (e.g., etrasimod, ozanimod), 5 aminosalicyclic acid and analogs thereof (such as, for example, mesalazine, sulfazaline, olsalazine, or balsalazide); probiotics (such as, for example, Saccharomyces boulardii), antibiotics (such as, for example, metronidazole ampicillin, ciprofloxacin, RHB-104), immunomodulators (such as, for example, tacrolimus, cyclosporine, methotrexate, thalidomide, leflunomide, and analogs of purines such as Azathioprine and 6-mercaptopurine), and stem cell therapy (e.g. darvadstrocel).

In one embodiment, the administration of the immune cell or population of the present disclosure allows reducing the amount of said at least one other active agent received by the subject.

According to one embodiment, the at least one immune cell population is administered before, at the same time or after the administration of the at least one other active agent.

### VI. Therapeutic Use

The present disclosure further relates to at least one isolated antibody binding to IL-23R as described herein or to at least one antigen-binding fragment of said antibody, for use as a medicament or for use in treating diseases, disorders, or symptoms in a subject in need thereof.

The present disclosure relates to at least one nucleic acid or vector as described herein, for use as a medicament or for use in treating diseases, disorders, or symptoms in a subject in need thereof.

The present disclosure further relates to a method for treating diseases, disorders, or symptoms in a subject in need thereof, comprising administering to the subject an isolated antibody or antigen-binding fragment thereof, a nucleic acid or a vector, or a composition, a pharmaceutical composition, or a medicament as described herein.

In one embodiment, the isolated antibody or antigen-binding fragment thereof, or the nucleic acid or the vector according to the present disclosure may be used for treating a disease or disorder mediated by IL-23R-expressing cells (also referred to herein as an IL-23R-related disease or disorder) in a subject in need thereof.

Another objective of the present disclosure is a cell therapy method for treating in a subject in need thereof a disease or disorder mediated by IL-23R-expressing cells, wherein said method comprises administering to the subject the immune cells described herein, e.g., the Treg cells described herein.

In one embodiment, the immune cells to be administered are autologous cells; in other words, the cell therapy is an autologous cell therapy. As used herein, the term “autologous” refers to any material derived from the same individual to whom it is later to be re-introduced.

In one embodiment, the cell therapy is a heterologous cell therapy. As used herein, the term “heterologous” refers to any material that is not derived from the subject to be treated but from an external source, e.g., induced pluripotent stem cells (iPSCs) or cells of cadaveric origin.

In one embodiment, the cell therapy is xenogeneic. As used herein, the term “xenogeneic” refers to any material derived from a subject of a different species as the subject to whom the material is introduced.

In another embodiment, the immune cells to be administered are allogenic cells; in other words, the cell therapy is an allogenic cell therapy. As used herein, the term “allogeneic” refers to any material derived from a different subject of the same species as the subject to whom the material is introduced. Two or more subjects are said to be allogeneic to one another when the genes at one or more loci are not identical. In a further embodiment, the immune cells are derived from a healthy human donor.

In some embodiments, the genetically modified immune cell of the invention may be an allogeneic immune cell. In such instances, the cell may be engineered to reduce host rejection to the cell (graft rejection) and/or the cell's potential attack on the host (graft-versus-host disease). By way of example, the cell may be engineered to have a null genotype for one or more of the following: (i) T cell receptor (TCR alpha chain or beta chain); (ii) a polymorphic major histocompatibility complex (MHC) class I or II molecule (e.g., HLA-A, HLA-B, or HLA-C; HLA-DP, HLA-DM, HLA-DOA, HLA-DOB, HLA-DQ, or HLA-DR; or 02-microglobulin (B2M)); (iii) a transporter associated with antigen processing (e.g., TAP-1 or TAP-2); (iv) Class II MHC transactivator (CIITA); (v) a minor histocompatibility antigen (MiHA; e.g., HA-1/A2, HA-2, HA-3, HA-8, HB-1H, or HB-1Y); and (vi) any combination thereof. The allogeneic engineered cells may also express an invariant HLA or CD47 to protect the engineered Treg cells from host rejection. These further genetic modifications may be performed by the gene editing techniques known in the art.

The further-edited allogeneic cells are particularly useful because they can be used in multiple patients without compatibility issues. The allogeneic cells thus can be called “universal” and can be used “off the shelf.”. The use of “universal” cells greatly improves the efficiency and reduces the costs of adopted cell therapy.

In certain embodiments, the allogeneic immune cell can be engineered such that it does not express any functional TCR on its surface, engineered such that it does not express one or more subunits that comprise a functional TCR or engineered such that it produces very little functional TCR on its surface. For example, an immune cell as described herein can be engineered such that cell surface expression of TCR molecules is downregulated. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

In certain embodiments, the allogeneic immune cell can be engineered such that it does not express a functional HLA on their surface. For example, an immune cell as described herein can be engineered such that cell surface expression of HLA, e.g., HLA class 1 and/or HLA class II and/or non-classical HLA molecules is downregulated.

In certain embodiments, the T cell can lack a functional TCR and a functional HLA such as HLA class I and/or HLA class II.

Modified immune cells that lack expression of a functional TCR and/or HLA can be obtained by any suitable means, including a knock out or knock down of one or more subunit of TCR and/or HLA. For example, the Treg cell can include a knock down of TCR and/or HLA using siRNA, shRNA, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain).

In some embodiments, the nucleic acid encoding a CAR as described herein is inserted at a specific locus in the genome of an immune cell, such as, for example, at the locus of a gene to be deleted. In some embodiments, the nucleic acid encoding a CAR as described herein is inserted within a TCR and/or HLA locus, thereby resulting in the inhibition of TCR and/or HLA expression.

Another objective of the present disclosure is a method for treating an IL-23R-related disease or disorder in a subject in need thereof, wherein said method comprises administering to the subject at least one CAR as described herein, or at least one nucleic acid or vector encoding a CAR as described herein. In one embodiment, the method is a gene therapy method.

### VII. IL-23R Related Diseases

In one embodiment, the IL-23R-related disease or disorder is a proinflammatory cell mediated disease or disorder, a Th17-mediated disease or disorder or a γδ T-mediated disease or disorder.

In one embodiment, the IL-23R-expressing cell-mediated disease is an autoimmune disease or disorder and/or an inflammatory disease or disorder.

Examples of IL-23R related diseases include, but are not limited to, autoimmune diseases or disorders, inflammatory diseases or disorders, allergic diseases or disorders, and cancer.

In one embodiment, said IL-23R-expressing cell-mediated disease or disorder is selected from inflammatory bowel disease (e.g., Crohn's disease and ulcerative colitis), lupus (e.g., systemic lupus erythematosus), arthritis (e.g., rheumatoid arthritis and juvenile idiopathic arthritis), Sjögren syndrome, systemic sclerosis, ankylosing spondylitis, type 1 diabetes, autoimmune thyroid disorders, multiple sclerosis, myasthenia gravis, psoriatic arthritis, skin diseases (e.g., psoriasis and atopic dermatitis), or uveitis.

In one embodiment, IL-23R-expressing cell-mediated disease or disorder is Crohn's disease.

### VIII. Article of Manufacture

Another object of the present disclosure is an article of manufacture containing materials useful for the treatment of a disease or disorder mediated by IL-23R-expressing cells.

The article of manufacture may comprise a container and a label or package insert on or associated with the container. Suitable containers include, for example, bags, bottles, vials, syringes, pouch, etc. The containers may be formed from a variety of materials such as glass or plastic

The article of manufacture, label or package insert may further comprise instructional material for administering the Treg cell population of the present disclosure to the patient.

The present disclosure provides a kit comprising at least one immune cell population of the present disclosure. By “kit” is intended to mean any article of manufacture (e.g., a package or a container) comprising at least one Treg cell population of the present disclosure. The kit may also contain instructions for use.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, cell biology, molecular described herein are those well-known and commonly used in the art. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this disclosure may be better understood, the following examples are set forth. These examples are for purposes of illustration only and are not to be construed as limiting the scope of the present disclosure in any manner.

### EXAMPLES

The present disclosure is further illustrated by the following examples.

### Example 1: Identification of an IL-23R-CAR Lead Candidate

### Materials and Methods

### PBMC Isolation

The blood of healthy donors was collected by the Etablissement Français du Sang (EFS). The day after blood collection, peripheral blood mononuclear cells (PBMC) were isolated from buffy coats by Ficoll gradient centrifugation, which enabled removal of unwanted fractions of blood product such as granulocytes, platelets and remaining red blood cell contaminants. Then, cell populations of interest were isolated as follow:

FOXP3 Treg and CD4⁺CD25⁻ Conventional T Cell Isolation

CD4⁺CD25⁺CD127^{low} Tregs were isolated using the Human CD4⁺CD127^{low}CD25⁺ Regulatory T Cell Isolation Kit (#18063; StemCell) following manufacturer's instructions. Briefly, CD25⁺ cells were first isolated from 400-500×10⁶ PBMC by column-free, immunomagnetic positive selection using EasySep™ Releasable RapidSpheres™. Then, bound magnetic particles were removed from the EasySep™-isolated CD25⁺ cells, and unwanted non-Tregs were targeted for depletion. The final isolated fraction contained highly purified CD4⁺CD127^{low}CD25⁺ cells that expressed high levels of FOXP3 and were immediately used for downstream applications. CD4⁺CD25⁻ conventional T cells were isolated by choosing the optional protocol for the isolation of CD4⁺CD25⁻ responder T cells from the kit #18063 (StemCell); for use in functional studies in parallel to Treg.

### Activation and Culture of Isolated Tregs

Isolated Treg cells were activated and cultured for 9 days. Briefly, at day 0, Treg cells (0.5×10⁶) were cultured into 24-well plate (Costar) with Xvivol5 serum-free medium containing human transferrin (OZYME) and supplemented with 1000 U/ml IL-2 (Euromedex) plus 100 nM rapamycin (Sigma-Aldrich). Then, CD3/CD28 activation was performed with Dynabeads® from Life Technology (0.5×10⁶ beads per well). At day 2, 4 and 7 cells were fed with fresh culture medium supplemented with 1000 U/ml IL-2. Finally, at day 9, cells were recovered, counted and reactivated.

### Lentiviral Vector Production and Titration

CAR-expressing lentiviral vectors (LVs) were produced using the classical 4-plasmid lentiviral system. Briefly, HEK293T cells (Lenti-X, Ozyme) were transfected with the CAR-expressing transfer vector, the plasmid expressing HIV-1 Gag/pol (pMDLg/pRRE), HIV-1 Rev (pRSV.Rev) and the VSV-G glycoprotein (pMD2.G) (Didier Trono, EPFL, Switzerland). 24-hours post-transfection, viral supernatants were harvested, concentrated by centrifugation, aliquoted and frozen at −80° C. for long term storage. The infectious titers expressed in transducing units per milliliter (TU/ml) were obtained after transduction of the Jurkat T cell line with a serial dilution of viral supernatants and transduction efficiency evaluated after 4 days by monitoring GFP expression.

### Transduction Protocol

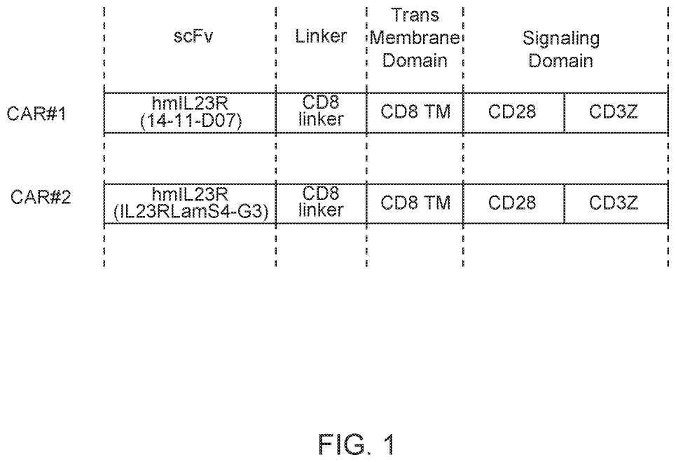

Tregs were transduced 2 days after their activation with a chimeric receptor (see schematic view of the CAR constructs in FIG. 1). Briefly, transduction was carried out by loading between 2 and 5×10⁶ Transducing Unit (TU) per ml to each well. After 6 hours at 37° C., viral particles were removed by washout. The plates were then incubated at 37° C. with 5% CO₂. Five days after the transduction, the transduction efficiency was analyzed: (i) the gene-transfer efficacy was measured by the analysis of the percentage of GFP positive cells in flow cytometry and (ii) the percentage of transduced cells which expressed the CAR at cell surface was measured by analyzing the hemagglutinin A (HA) tag expression or protein-L staining in flow cytometry.

### CAR Constructs Used for Transduction

IL-23R CARs composed of the CD8 transmembrane (TM) and intracellular domain of CD28 in tandem with the intracellular domain of CD3 zeta and associated with scFv directed against IL-23R were designed. The constructs used in this study are listed and described in FIG. 1.

### Phenotype Analysis of Transduced Tregs

At day 9 of the culture, Treg phenotype was analyzed by flow cytometry using the markers listed in Table 1.

TABLE 1
Materials and reagents used for Treg immunophenotyping

| Reagents | Manufacturer | Reference |
|---|---|---|
| CD4 VioGreen | Miltenyi | 130-096-900 |
| Helios eF450 (HamIgG) | eBioscience | 48-9883-42 |
| CD25 PE | Miltenyi | 130-109-020 |
| CD152 (CTLA-4) PECy7 (mIgG2a) | Biolegend | 369614 |
| FOXP3 AF647 (mIgG1) | BD | 560045 |
| CD127-APC-Vio770 | Miltenyi | 130-109-438 |

### Activation Assay of CARs

Activation assay was performed at day 9 of the culture. Briefly, 0.05×10⁶ Treg were seeded in 96-well U bottom plates alone or in the presence of anti-CD3/anti-CD28 coated beads (in a 1:1 Treg to beads ratio), or in the presence of IL-23R coated beads (in a 1:1 Treg to beads ratio) in a 200 μL final volume. After 24 h at 37° C., 5% CO2, cells were stained for CD4 and CD69 and then analyzed using flow cytometry. Compared to untransduced Treg cells, the monitoring of the CD69 spontaneous expression in CAR Treg cells, allows us to determine the tonic signaling intensity.

### Suppression Assay of T Cell Proliferation

The suppressive assays were performed at day 9 of the culture. Briefly, Treg were recovered, counted and activated either through the TCR using anti-CD3/anti-CD28 coated beads (in a 1 to 1 Treg to beads ratio), or through the CAR IL-23R coated beads (in a 1 to 1 Treg to beads ratio) or kept without activation to evaluate their spontaneous suppressive activity. In parallel, allogeneic Tconv were thawed, stained with Dye 450 and activated with anti-CD3/anti-CD28 coated beads (in a 3 to 1 Tconv to beads ratio). The day after, beads were removed from Tconv before their coculture with un-activated or activated Treg (non-transduced or transduced). At day 3, cells were harvested, and the proliferation of Tconv was assessed by flow cytometry through the determination of dye 450 dilution. The percentage of inhibition of Tconv proliferation was calculated as followed:

$$100 - \frac{\% of Tconv proliferation in presence of CAR - Treg \times 100}{\% of Tconv proliferation in absence of CAR - Treg}$$

### Animals

All procedures described in this study have been reviewed and approved by the local ethics committee (CIEPAL). The experiments were performed on 8-week-old female LY5.1 mice. Mice were hosted in groups of six individuals, with each mouse uniquely identified. Animals were housed in ventilated cages (type II (16×19×35 cm, floor area=500 cm²)) under the following controlled conditions: (i) room temperature (22±2° C.), (ii) hygrometry (55±10%), (iii) photoperiod (12:12-hour light-dark cycle 7 am:7 pm), and (iv) water and food (Ref. 2018, Harlan France) available ad libitum. Mice were allowed to acclimate to the environment for five days prior to the beginning of the experiment.

### DSS Induced Acute Colitis

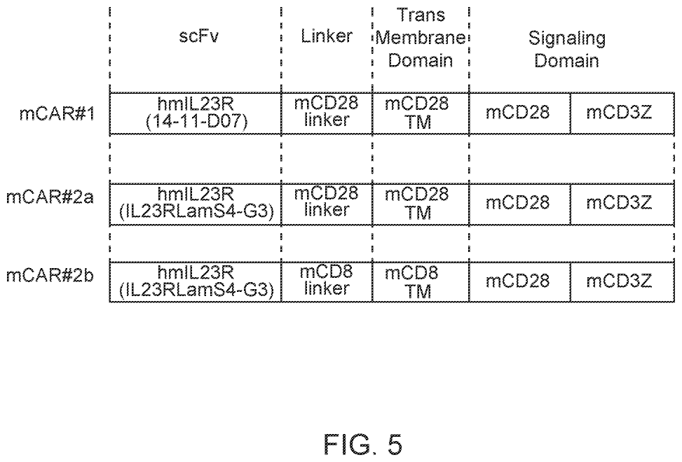

DSS (Dextran Sulfate Sodium, 40 kDa, MP Biomedicals) was administered to 8-week-old female LY5.1 mice during days 0 to 5 (D0-D5). DSS (two different concentrations) was added in drinking water (ad libitum) freshly renewed every two days. Fresh water without DSS was provided during the recovery period (D5 to D15). At day 5, three million CAR positive Tregs isolated from the spleens of 10-week-old C57BL/6J mice were injected by caudal IV. The CARs used to transduce the mouse Tregs are described in FIG. 5. Mice were scored daily based on four parameters: body weight loss, stool consistency, blood presence, and time to collect a stool, as noted below in Table 2.

TABLE 2
Parameters used for disease activity index

| | | |
|---|---|---|
| Body weight loss | <5% | 0 |
| | 5-10% | 1 |
| | 10-20% | 2 |
| | >20% | 3 |
| Stool consistency | Normal stool | 0 |
| | Slightly soft stool | 1 |
| | Soft stool by formed | 2 |
| | Unformed stool | 3 |
| | Watery stool | 4 |
| Blood presence | No | 0 |
| | Weak appearance of blood | 1 |
| | Visual blood in stool 50% | 2 |
| | Visual blood in stool 75% | 3 |
| | Visual blood in stool 100% | 4 |

| | | | |
|---|---|---|---|
| Time to collect a stool | <5 | min | 0 |
| | 5-10 | min | 1 |
| | 10-15 | min | 2 |
| | 15-20 | min | 3 |
| | >20 | min | 4 |

Stools were collected for inflammatory marker analysis. At day 15, the mice were euthanized by cervical dislocation and organs were collected for ex vivo analysis.

### Ex Vivo Analysis

### Tissue Digestion

Colon was washed in PBS to remove fecal content, then incubated in PBS 5 mM EDTA for 30 min at 37° C. The colon was digested in RPMI media with 2 mg/mL collagenase D and 20 μg/mL DNAse for 30 min at 37° C. The colon was then mashed through a cell strainer to obtain a single cell suspension. Cells were then washed and resuspended in 3 mL PBS-2% FCS, and 200 μL were plated for antibody staining.
Spleen and mesenteric lymph node were mashed through a cell strainer to obtain single cell suspensions. Red blood cells were lysed with 1 mL red blood cell lysis buffer for 2 min. Cells were then washed and resuspended in 5 mL and 1 mL of PBS-2% FCS, respectively, and 200 μL were plated for antibody staining.

### ELISA for Lipocalin Detection in Mice Feces

Feces were collected and frozen at −20° C. every day. The fecal samples were resuspended in 1 mL of PBS Tween 0.1% and centrifuged 20 min at 4000 rpm. Supernatants were harvested and plated at 1/1000 for lipocalin detection using DuoSet ELISA Mouse Lipocalin-2/NGAL (DY1857-05) according to the manufacturer's instructions.

### Multicolor Flow Cytometric Analysis

Cell subsets in organs were stained for flow cytometric analysis as follows:

TABLE 3
Materials and reagents used for flow cytometric analysis of organ cell subsets

| | | Concentration | Working | |
|---|---|---|---|---|
| | Cell Surface Antibody | Stock | Concentration | Ref. |
| Panel 1 | Dump Channel PE (CD3, | 0.2 mg/mL | 1/500 | B220: 103208 (bioleg) |
| myeloid | CD19, NK1.1) | 0.2 mg/mL | | Nk1.1: 108708 (bioleg) |
| | | 0.2 mg/mL | | CD4: 553048 (BD) |
| | | 0.2 mg/mL | | CD8: 553032 (BD) |
| | IL23R BV421 | 0.2 mg/mL | 1/100 | 150907 (bioleg) |
| | LY6G APC | 0.2 mg/mL | 1/400 | 127614 (bioleg) |
| | CD11c FITC | 0.5 mg/mL | 1/100 | 117306 (bioleg) |
| | LY6C BV510 | | 1/200 | 128033 (bioleg) |
| | CD11b PE Efluor 610 | 0.2 mg/mL | 1/100 | 61-0112-82 (Thermo) |
| | CD45 Alexa Fluor 700 | 0.5 mg/mL | 1/200 | 103128 (bioleg) |

| | | Intracellular | | | |
|---|---|---|---|---|---|
| | Cell Surface Antibody | Antibody | Dilution | Ref. | |
| Other | CD45.2 APC | | 1/200 | 558702 | (BD) |
| Panel | TCR abeta BV510 | | 1/100 | 563221 | (BD) |
| | B220 PE | | 1/200 | 103208 | (bioleg) |
| | CD4 BV605 | | 1/200 | 116027 | (bioleg) |
| | CTLA4 PE CF594 | CTLA4 PE | 1/200 | 562742 | (BD) |
| | | CF594 | | | |
| | IL23R BV421 | | 1/100 | 150907 | (Bioleg) |
| | NGFR AF488 | | 1/100 | 53940042 | (Thermo) |
| | | Ki67 PECy7 | 1/200 | | |
| | CD45.1 APC Cy7 | | 1/200 | 110716 | (bioleg) |

### Results

### Transduction Efficiency and CAR Expression at the Cell Surface

Transduction efficiency was assessed by the percentage of GFP positive cells and CAR expression was monitored using recombinant protein-L, an immunoglobulin kappa light chain-binding protein or an antibody directed against HA Tag. Results for the percentage of transduction efficiency and the percentage of transduced cells which expressed the CAR at their cell surface are provided in Table 4. Both constructs conferred more than 95% expression of the CAR at the cell surface. Furthermore, the mean fluorescence intensity (MFI) representing the number of CARs per cell is comparable.

TABLE 4
Transduction efficiency and CAR expression

| CAR constructs | CAR#1 | CAR#2 |
|---|---|---|
| Donors | n = 5 | |
| TU/ml for transduction | 5 × 10⁶ | |
| % of Transduction efficacy | 47.8 ± 3.6 | 67.27 ± 1.7 |
| % of CAR expression at cell surface | 95.8 ± 0.63 | 97.3 ± 0.3 |
| MFI of the CAR | 29.08 ± 4.1 | 29.8 ± 3.4 |

### Stable Treg Phenotype is Observed in the Presence of the New Anti-IL23R CAR #2

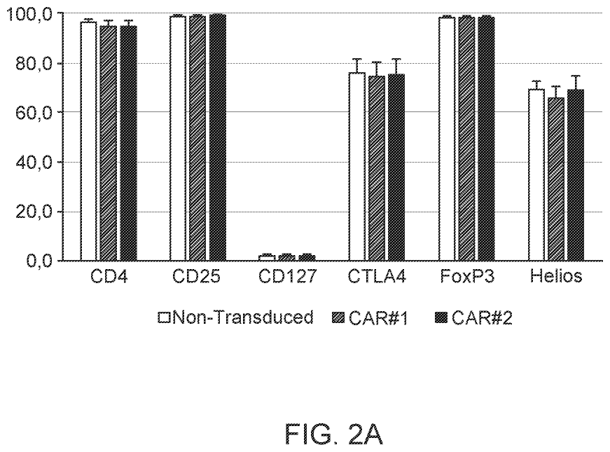

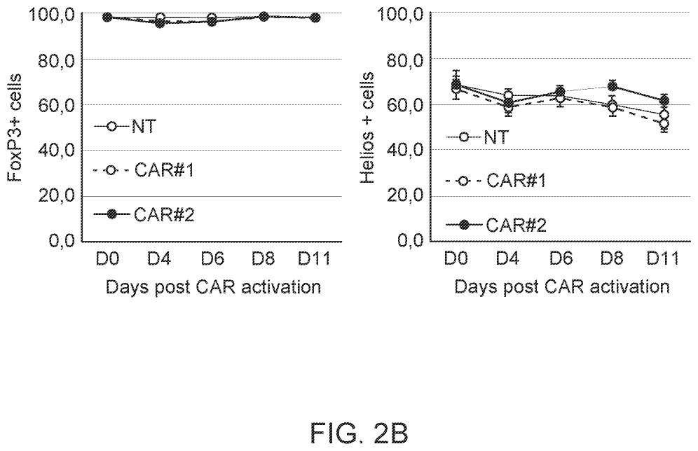

A major challenge with engineered T cells in general is to ensure the maintenance of the desired phenotype especially since it has been shown that high expression of CARs has been linked to undesired antigen-independent CAR activation (Frigault et al., Cancer Immunol Research (2015) 3(4):356-67). To evaluate whether the Treg phenotype is altered during expansion and CAR engagement, a panel of markers related to Treg identity was analyzed. The maintenance of Helios and FOXP3 expression and other markers associated to Treg phenotype was evaluated on FOXP3⁺ Tregs. IL-23R CAR-Tregs maintained high expression of FOXP3 and Helios after expansion, at Day 9 (FIG. 2A), and remained stable until 11 days after CAR engagement using IL-23R coated beads (FIG. 2B).

New scFv-Derived CARs Maintain CAR-Specific Activation

The CAR #2 construct allowed for a reduction of the activation background of Treg cells as compared to the CAR construct control (CAR #1). As shown in FIG. 3, the percentage of CD69⁺IL-23R CAR-Treg cells in the absence of activation was much lower with CAR #2, as compared to CAR #1, which led to 60% CD69⁺ cells. In addition, IL-23R CAR-Treg cell activation using anti-CD3/anti-CD28 coated beads induced a 6.41-fold increase in CD69⁺ cells with CAR #2 and only 1.52-fold increase with CAR #1.

Finally, IL-23R CAR-Treg cell activation using IL-23R coated beads was increased by 3.6-fold with CAR #2, while it was increased by only 1.33-fold with CAR #1. These results show the superior CAR-mediated activation of Treg cells expressing the CAR construct comprising the scFv of the present disclosure.

### CAR #2 Exhibits Efficient CAR-Mediated Suppressive Activity

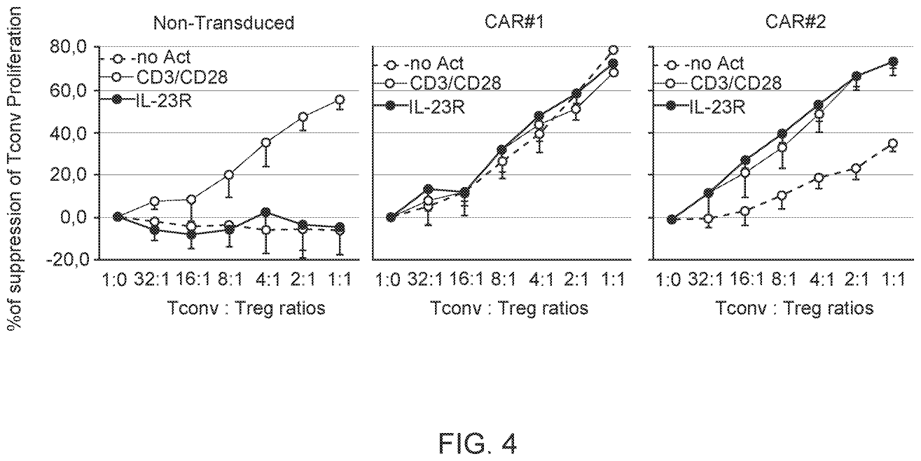

For CAR #2, we observed a CAR-specific triggering of the suppressive activity compared to the IL-23R CAR construct harboring the control scFv (CAR #1) (FIG. 4). Indeed, as shown in FIG. 4, the spontaneous suppressive activity of CAR #1-expressing Treg cells (No Act condition) was too strong to highlight a specific TCR or CAR-mediated suppressive activity. By contrast, stimulation of CAR #2-expressing Treg cells allowed a potent suppressive activity either after TCR engagement or after CAR #2 activation using IL-23R ligand. These results highlight the superiority of the scFv of the present disclosure.

Tregs with New scFv-Derived CARs are Activated In Vivo and Reduce the Disease Activity Index in a DSS-Induced Mouse Model of IBD

Mouse Treg cells were transduced with the different constructs described in FIG. 5. Two versions of mouse CAR #2CD28 (mCAR #2) comprising the new scFv (which is cross-reactive with human and mouse) have been built: (1) mCAR #2a, which comprises the same TM domain as mCAR #1 (mouse CD28 TM); and (2) mCAR #2b, which comprises the TM domain of mouse CD8 (more comparable to the human construct CAR #2).

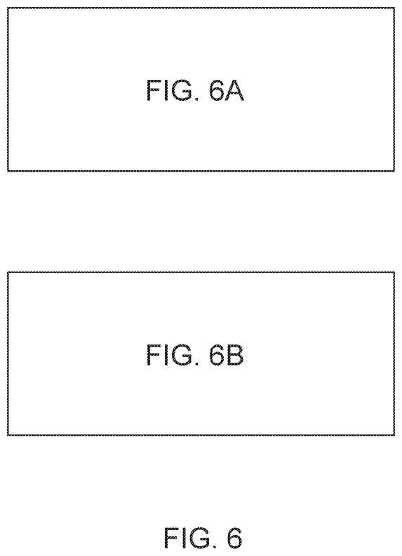

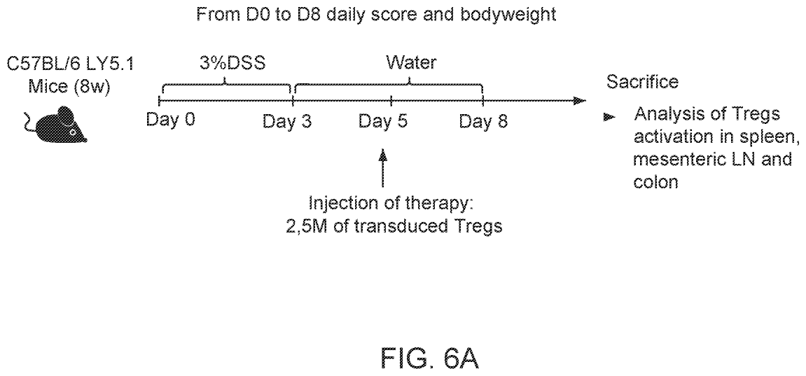

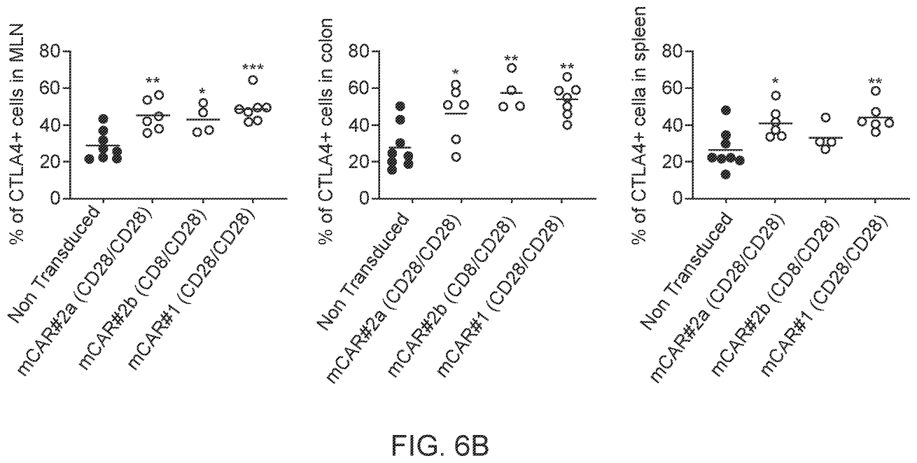

Non transduced (NT) or IL-23R-CAR mouse Tregs, transduced with mouse CAR constructs either derived from the control scFv (mCAR #1) or the scFv of the present disclosure (mCAR #2a and 2b), were injected into a short dextran sodium sulfate (DSS) induced mouse model of inflammatory bowel disease (IBD) (FIG. 6A). This model was developed to measure the activation in situ of the injected cells, and is a target engagement model rather than an efficacy model. As shown in FIG. 6B, IL-23R CAR Tregs comprising the scFv of the present disclosure (mCAR #2) are found in vivo in colon, mesenteric lymph nodes and spleen. These Tregs are significantly more activated than non-transduced cells at sites of inflammation (in colon and mesenteric lymph nodes), as measured by the expression of CTLA-4 on Tregs using flow cytometry. Interestingly, mCAR #2b (comprising a CD8 TM associated with a CD28 intracellular signaling domain) showed significant activation only in inflamed organs (colon and mesenteric lymph nodes) and not in a non-inflamed organ (spleen).

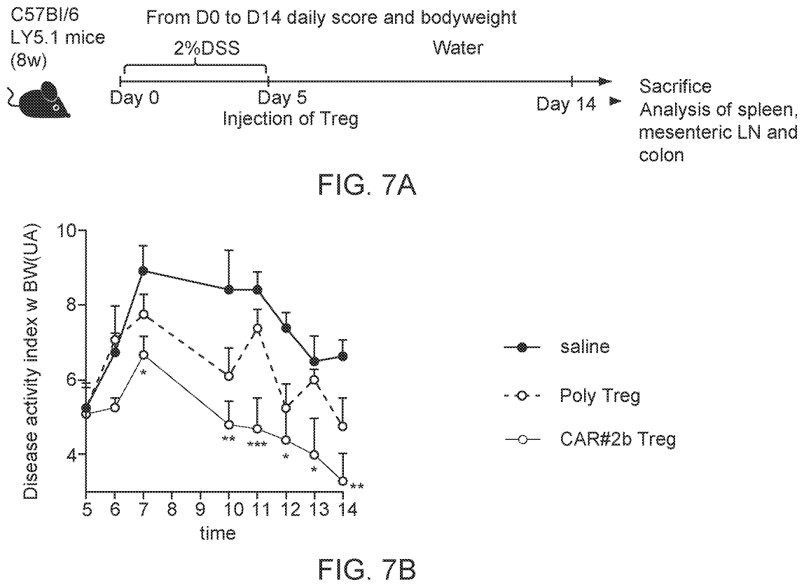

Upon testing of the Tregs in an efficacy model of DSS-induced IBD (FIG. 7A), mCAR #2 was shown to induce a significant reduction in the disease activity index (FIG. 7B).

### Discussion

CAR #2, an IL-23R-CAR lead candidate (LamS4G3) was identified. CAR #2 is a 2nd generation CAR, composed of an anti-IL-23R-scFv identified through a large library screening which obtained more than 100 potential hits, fused to CD8TM/CD28/CD3z. This CAR showed high specificity to IL-23R with an undetectable tonic signaling, and a specific CAR-dependent suppressive activity in vitro.

### Example 2: In Vitro Selection in Human Treg of Optimized IL-23R CAR Construct

Starting from CAR #2 identified in Example 1, several optimizations were done by varying the promotor, leader sequence, hinge-length and codon optimization. 6 different optimized constructs were selected out of a 32-constructs-matrix and produced in clinical-ready backbone (without HA-tag and GFP).

In this Example, those different constructs were compared based on several parameters, including viability, fold expansion, Treg phenotype stability, CAR-mediated activation, and CAR-mediated suppression.

### Materials and Methods

### Generation of IL-23R Expressing Jurkat Cells Lines

Jurkat IL23R-cell lines were generated by overexpressing IL23R in Jurkat cells via a puromycin selectable, lentiviral vector. Lentiviral vectors were produced and transduced in Jurkat cells.

Generation of Artificial APC Presenting rhuIL-23R

Dynabeads (M-270 Epoxy; ThermoFisher Scientific) were coupled with Recombinant Human IL-23R Fc Chimera Protein (R&D, catalog number 1400-IR-050) with the Dynabeads Antibody Coupling Kit (Life technology, Thermo fisher, catalog number 14311D) following the manufacturer's instructions.

### PBMC and Regulatory T Cells Isolation

Tregs and Tconv were freshly isolated from buffy coats obtained from heathy volunteer bloods from EFS (Marseille). Briefly, the day after the blood donation, peripheral blood mononuclear cells (PBMC) were isolated from buffy coats by Ficoll gradient centrifugation. CD4+/CD25+/CD127low nTreg cells were isolated following the procedure: CD25+ cells are isolated by column-free immunomagnetic positive selection using EasySep™ Releasable RapidSpheres™. Then, bound magnetic particles were removed from the EasySep™-isolated CD25+ cells, and unwanted non-Tregs were targeted for depletion. The final isolated fraction contained highly purified CD4+CD127lowCD25+ cells that express high levels of FOXP3.

### Transduction

Constructs used in this study are described in Table 5.

TABLE 5
Anti-IL-23R chimeric antigen receptor (CAR) constructs

| | | | Co-signaling | | Promoter/Post- | |
|---|---|---|---|---|---|---|
| | scFv | | and signaling | Leader | transcriptional | |
| Name | name | TM | domain | peptide | regulator | Linker |
| CAR#2 | hmIL23R | CD8 | CD28 + | CD8 | PGK/WPRE- | STD |
| CAR#3 | LamS4-G3 | | CD3 zeta | CD25 | PGK/WPRE | STD |
| CAR#4 | | | | CD8 | PGK/WPRE | STD |
| CAR#5 | | | | Igk | PGK/WPRE | STD |
| CAR#6 | | | | CD8 | PGK/— | STD |
| CAR#7 | | | | CD8 | PGK/WPRE | STD |
| CAR#8 | | | | CD8 | PGK/— | STD |
| CAR#9 | | | | CD8 | PGK/WPRE | STD |
| CAR#10 | | | | CD8 | EF1a/— | STD |
| CAR#11 | | | | CD8 | EF1a/WPRE | STD |

The transduction was done in 24-well plate. Briefly, at day 2 after isolation and activation, 2/3 of media was removed from each well and 2-5×10E6 TU/ml of each construct was added per well. After 6 hours at 37° C., 2/3 of fresh medium were added per well and each well were homogenized, recovered in tube and centrifugated. Supernatant were removed, and each pellet were resuspended in fresh medium supplemented with 1000 U/ml of L-2 before to be seeded in a new plate. 4-5 days after the transduction; the efficacy was measured by the analysis of the percentage of IgG* positive cells in flow cytometry.

At day 9 of the culture, Treg phenotype was analyzed following the procedure STF-TRB-01-E009 V02. Markers used for this analysis are listed in the updated Table 6.

TABLE 6
Markers used for Treg immunophenotyping

| Panel | Designation | Manufacturer | Reference |
|---|---|---|---|
| Treg status | CD4 VioGreen | Miltenyi | 130-096-900 |
| | Helios eF450 | eBioscience | 48-9883-42 |
| | CD25PE | Miltenyi | 130-109-020 |
| | CTLA-4 - PECy7 | Biolegend | 369614 |
| | FoxP3 AF647 | BD | 560045 |
| | CD127-APC-Vio770 | Miltenyi | 130-109-438 |

### Activation Assay

At day 10 of the culture, activation assay was performed. Briefly 0.05×10\hat{ }6 CAR-Treg were seeded in PL96 U bottom alone or in presence of anti-CD28/anti-CD3 coated beads (1 to 1 Treg to beads ratio), or in presence of IL-23R coated beads (1 to 1 Treg to beads ratio) or in presence of Jurkat expression low (Jurkat 572) or high level of IL-23R (Jurkat 573) at cell surface in 200 μl final volume. After 24 h at 37° C., 5% CO2, cells were stained for CD4 and CD69 and then analyzed in flow cytometry.

### Suppression Assay

The suppressive assays were performed at day 10, CAR-Treg were harvested counted, and activated either through the TCR using anti-CD28/anti-CD3 coated beads (in a 2 to 1 Treg to beads ratio), or through the CAR using either IL-23R coated beads (in a 1 to 1 Treg to beads ratio) or Jurkat cells lines expressing low (Jurkat 572) or High (Jurkat 573) level of IL-23R at cells surface, or kept without activation to evaluate their basal in term of functional activity. In parallel, allogeneic Tconv were thawed, stained with Dye 450 and activated with anti-CD28/anti-CD3 coated beads (in a 3 to 1 Tconv to beads ratio). The day after beads were removed from Tconv before their coculture with un-activated or activated Treg. After 3 days of coculture the proliferation of Tconv was analyzed in flow cytometry.

### Results

### Comparable Transduction Efficiency

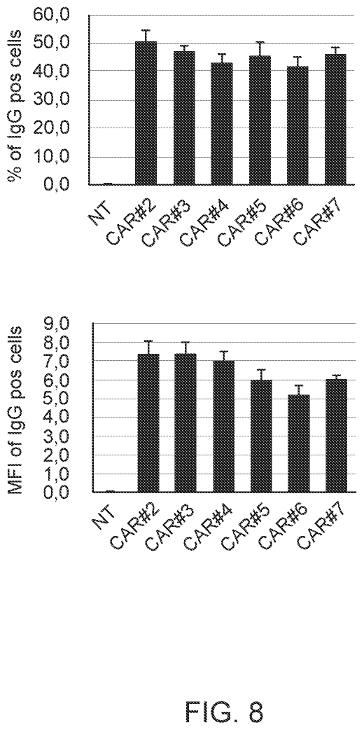

Selected CARs covering CAR-expression levels from Low to High, have no detectable background/tonic signaling, and are covering different biological elements (Signal peptide/Codon/linker/WPRE). The constructs used in that study were generated and produced in clinical-ready backbone (without HA-tag and GFP). The transduction efficiency was assessed in flow cytometry measuring the percentage of cells positive for IgG using an anti-IgG antibody and the level of CAR expression at cell surface was evaluated by the mean fluorescence intensity (MFI) of the IgG staining (FIG. 8). The different constructs were transduced between 40-50%.

The Different Optimized Constructs Highlight the Same Viability and Fold Expansion after the First Cycle in Culture

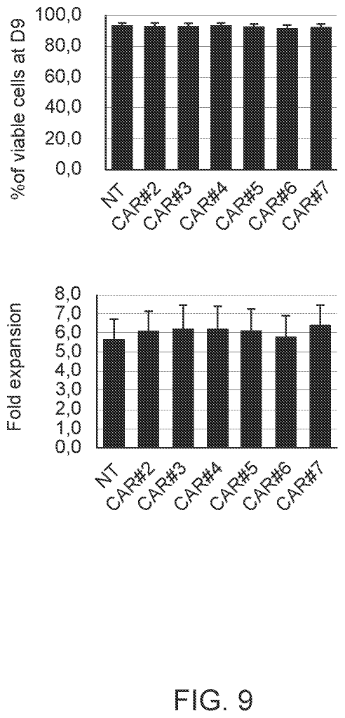

The impact of the different changes on the behavior of the CAR-Treg in culture was also assessed. Whatever the changes, Treg transduced with the different constructs showed the same viability level (FIG. 9, on the left) and fold of expansion (FIG. 9, on the right) at day 9.

CAR #3 showed the lowest background of activation and the highest signal versus noise after CAR activation.

The impact of the different optimization on the activation of the CAR-Treg (FIG. 10) was evaluated. The profile of the CAR-mediated activation for CAR #3, CAR #4, CAR #5, CAR #6, and CAR #7 were close to the one of the non-optimized construct (CAR #2). However, the background of activation of CAR #3 was slightly lower than that of CAR #2 highlighting the best signal versus noise.

### Significant CAR Mediated Suppression

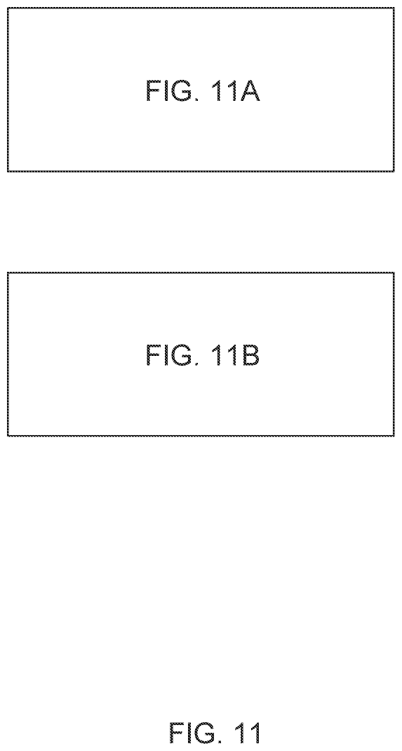

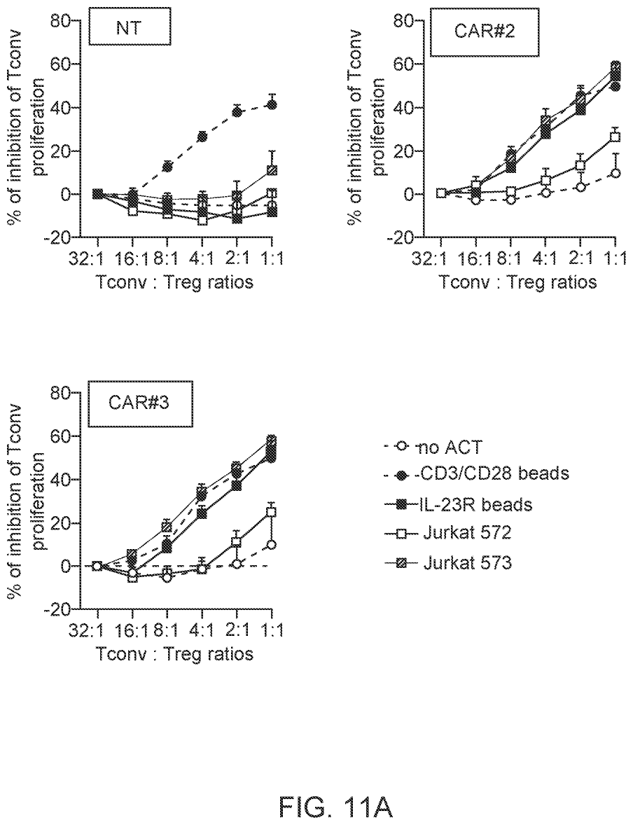

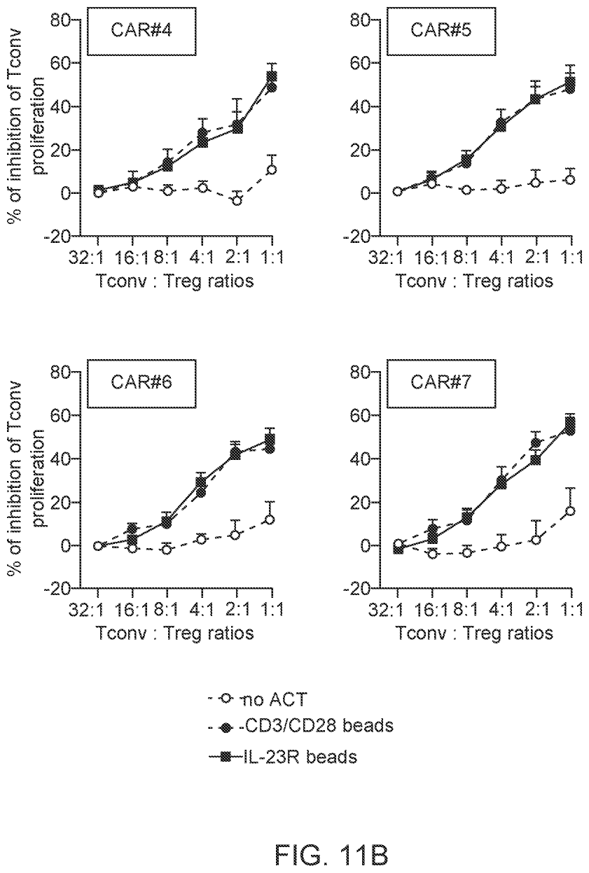

The CAR-mediated suppressive activity of the CAR constructs was measured (FIG. 11). The CAR engagement was induced using either artificial APC (IL-23R coated beads; bIL-23R), or low expressing (Jurkat 572) or high expressing (Jurkat 573) Jurkat cells lines. As positive control we used polyclonal activation (anti-CD3/anti CD28 coated beads; beads CD3/28). The spontaneous suppression is measured without any activation (none). Like for the activation data, the profiles for CAR #3, CAR #4, CAR #5, CAR #6, and CAR #7 are close to the one of the non-optimized construct CAR #2. All constructs are able to mediate a robust CAR mediated suppressive activity when the CAR is engaged using artificial APC (FIGS. 11A and 11B) or high expressing IL-23R Jurkat cell line (FIG. 11A). All constructs highlighted a very low spontaneous suppressive activity.

The Area Under the Curve was calculated for CAR #2 and CAR #3 (FIG. 12). The AUC calculation highlights that CAR #3 showed the same CAR mediated suppression as CAR #2 when Treg are engaged with Jurkat expressing high level of IL-23R at cell surface (Jurkat 573). However, CAR #3 showed lower CAR mediated suppression than CAR #2 when CAR Treg were engaged with Jurkat expressing low level (Jurkat 572). This profile should reduce off target effect.

### Treg Stability—all Constructs Showed a Good Stability of the Treg Phenotype

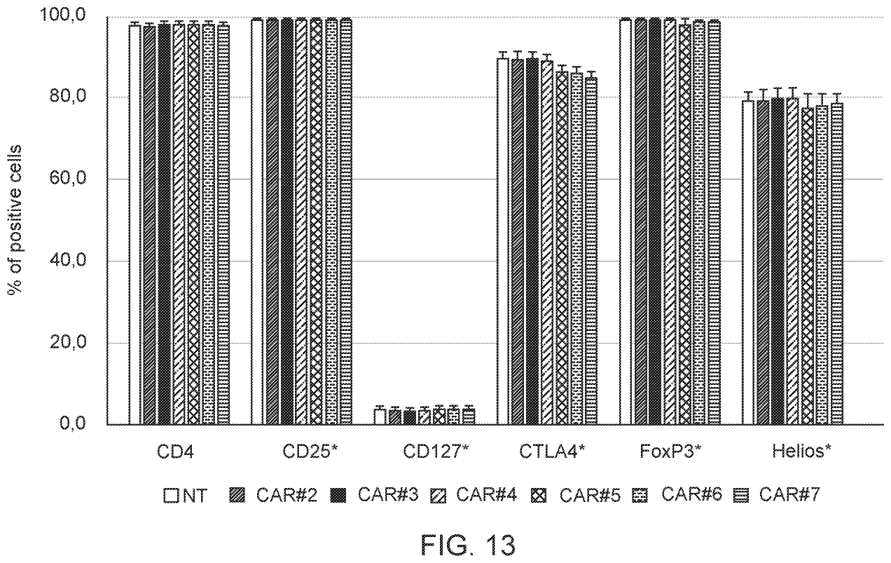

Plasticity is a well described feature of Tregs. The main markers of Treg identity are CD4, CD25, CTLA-4, FoxP3 and Helios. To measure the phenotypic stability of CAR Tregs, the expression of these markers was analyzed by flow cytometry at the end of the first cycle of expansion (Day 9) (FIG. 13). No modification of the main Treg markers was observed following the different CAR used.

### Discussion

The differences between the different constructs are very slight highlighting that the standard CAR #2 is robust enough to assume the different changes.

However, the CAR #3 construct stood out based on the following parameters:
Lower tonic signaling than CAR #2 leading to a higher signal versus noise (FIG. 10)
Same CAR mediated suppression as CAR #2 when Treg are engaged with Jurkat expressing high level of IL-23R at cell surface whereas lower CAR mediated suppression than CAR #2 when CAR Treg were engaged with Jurkat expressing low level of IL-23R (FIG. 12). This should reduce off target.

### Example 3: IL23R-CAR: ScFv (LamS4G3) Affinity Characterization

### Materials and Methods

The indicated scFVs were produced in mammalian 293tcells, purified and biotinylated. Biotinylated scFvs were then incubated at 25° C. with mouse or human IL23Ra-coated beads for 30 min in PBS. After cold washing, bound IL23R bound scFV was detected by incubation with APC-labelled Streptavidin and analysis of the samples on a Flow-cytometer. An EC50 was calculated based on the median fluorescence intensity from each condition. Calculation, curve fitting and EC50 calculation was performed via Graphpad Prism.

### Results

Binding experiments revealed a high affinity binding to human IL23R, with an EC50 of 37.6 nM, and an almost similar affinity to mouse IL23Ra, with an EC 50 of 53.1 nM. In contrast the reference scFv, being also cross-reactive resulted in a higher affinity binding of 4.4 Nm to human Il23R and a more pronounced difference of 13.1 nM to mouse IL23R (FIG. 14).

In these exemplary results, a CAR of the present disclosure exhibit a similar binding profile between the human and the mouse homologue of IL23R. In these exemplary results, CAR #2 of the present disclosure demonstrates about a 1.4-fold difference in binding affinity between human IL23R and mouse IL23R, whereas CAR #1 demonstrates about a 3-fold difference in binding affinity between human IL23R and mouse IL23R. These exemplary results show that CAR #2 of the present disclosure binds to human and mouse IL23R with a more similar affinity as compared to CAR #1. This similarity indicates that the use of CAR #2 in a mouse preclinical model may be more representative of its interaction with human IL23R.

### Example 4: Stability of scFv Signaling

### Materials and Methods

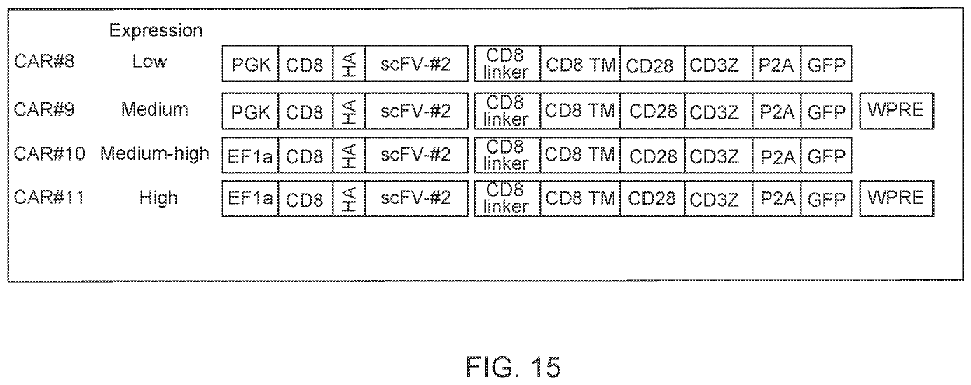

Four (4) different constructs were generated with different expression cassettes (PGK or EF1a promoters, +/−WPRE Mut6) and transduced into primary human Treg by lentiviral transduction (FIG. 15).

### Results

The selected IL-23R scFv demonstrated stable signaling and low background under similar expression levels in primary Treg cells (FIG. 16).

Increased expression correlates with increased accumulation of CAR at cell surface. While expression levels varied by a factor of 5, no difference in background over unmodified control cells (horizontal dark grey line in CAR Tonic Signaling graph) was observed (FIG. 16). Additionally, all CAR expressing conditions reacted similarly strong to CAR dependent activation, as well as to control activation with CD3/CD28 beads (FIG. 16).

TABLE OF SEQUENCES

| SEQ | | |
|---|---|---|
| ID | Sequence | |
| NO | function | Sequence |
| 1 | VH CDR1 | SSNYYWG |
| 2 | VH CDR2 | GSIYYSGNTYYNPSL |
| 3 | VH CDR3 | REWSPYESEGFDY |
| 4 | VL CDR1 | TGSSSNIGAGYDVH |
| 5 | VL CDR2 | GNNNRPS |
| 6 | VL CDR3 | QSYDTGLSAW |
| 7 | VH chain | QLQLQESGPGLVKPSETLSLTCTVSGGSITSSNYYW |
| | | GWIRQPPGKGLEWIGSIYYSGNTYYNPSLKSRVTIS |
| | | VDTSKNQFSLKLSSVTAADTAVYYCAREWSPYESE |
| | | GFDYWGQGTLVTVSS |
| 8 | VL chain | QSVLTQPPSVSGAPGQRVTISCTGSSSNIGAGYDVH |
| | | WYQQLPGTAPKLLIYGNNNRPSGVPDRFSGSKSGTS |
| | | ASLAITGLQAEDEADYYCQSYDTGLSAWVFGGGTK |
| | | LTVL |
| 9 | (G3S)-Linker | GGGS |
| 10 | (G3S)4-Linker | GGGSGGGSGGGSGGGS |
| 11 | (G4S)-Linker | GGGGS |
| 12 | (G4S)2-Linker | GGGGSGGGGS |
| 13 | (G4S)3-Linker | GGGGSGGGGSGGGGS |
| 14 | (G4S)4-Linker | GGGGSGGGGSGGGGSGGGGS |
| 15 | scFv | QSVLTQPPSVSGAPGQRVTISCTGSSSNIGAGYDVH |
| | | WYQQLPGTAPKLLIYGNNNRPSGVPDRFSGSKSGTS |
| | | ASLAITGLQAEDEADYYCQSYDTGLSAWVFGGGTK |
| | | LTVLGGGGSGGGGSGGGGSQLQLQESGPGLVKPSE |
| | | TLSLTCTVSGGSITSSNYYWGWIRQPPGKGLEWIGSI |
| | | YYSGNTYYNPSLKSRVTISVDTSKNQFSLKLSSVTA |
| | | ADTAVYYCAREWSPYESEGFDYWGQGTLVTVSS |
| 16 | VH coding | CAGCTGCAGCTACAGGAGAGCGGCCCCGGCCTG |
| | sequence | GTGAAACCTTCTGAGACCCTGAGCCTGACGTGCA |
| | | CCGTGAGCGGCGGCAGCATCACCAGCAGCAACT |
| | | ACTACTGGGGCTGGATACGACAGCCCCCTGGAAA |
| | | GGGCCTAGAGTGGATCGGCAGCATCTACTACAGC |
| | | GGCAACACCTACTACAACCCCAGCCTGAAGAGCA |
| | | GAGTCACCATCAGCGTGGACACCAGCAAGAACC |
| | | AGTTCAGCCTGAAGCTGAGCAGCGTGACCGCCGC |
| | | CGACACAGCTGTTTACTATTGTGCCAGAGAGTGG |
| | | AGCCCCTACGAGAGCGAGGGCTTCGACTACTGGG |
| | | GCCAGGGGACCCTGGTGACCGTTTCTTCT |
| 17 | VL coding | CAGAGCGTGCTGACACAGCCTCCATCTGTTTCTG |
| | sequence | GCGCCCCTGGCCAGAGAGTGACCATCTCTTGTAC |
| | | TGGCAGCAGCTCCAACATTGGAGCCGGCTATGAC |
| | | GTGCACTGGTACCAgCAGCTGCCTGGCACAGCCC |
| | | CTAAACTGCTGATCTACGGCAACAACAACAGACC |
| | | CAGCGGCGTGCCCGACAGATTTTCTGGCTCCAAG |
| | | AGCGGCACAAGCGCCAGCCTGGCTATTACTGGAC |
| | | TGCAGGCCGAGGACGAGGCCGACTACTACTGTCA |
| | | GAGCTACGACACCGGCCTGAGCGCCTGGGTTTTT |
| | | GGTGGAGGCACAAAACTGACAGTGCTG |
| 18 | (G4S)3-linker | GGAGGCGGAGGTTCTGGCGGCGGAGGAAGTGGT |
| | coding sequence | GGCGGAGGCTCA |
| 19 | scFv coding | CAGAGCGTGCTGACACAGCCTCCATCTGTTTCTG |
| | sequence | GCGCCCCTGGCCAGAGAGTGACCATCTCTTGTAC |
| | | TGGCAGCAGCTCCAACATTGGAGCCGGCTATGAC |
| | | GTGCACTGGTACCAGCAGCTGCCTGGCACAGCCC |
| | | CTAAACTGCTGATCTACGGCAACAACAACAGACC |
| | | CAGCGGCGTGCCCGACAGATTTTCTGGCTCCAAG |
| | | AGCGGCACAAGCGCCAGCCTGGCTATTACTGGAC |
| | | TGCAGGCCGAGGACGAGGCCGACTACTACTGTCA |
| | | GAGCTACGACACCGGCCTGAGCGCCTGGGTTTTT |
| | | GGTGGAGGCACAAAACTGACAGTGCTGGGAGGC |
| | | GGAGGTTCTGGCGGCGGAGGAAGTGGTGGCGGA |
| | | GGCTCACAGCTGCAGCTACAGGAGAGCGGCCCC |
| | | GGCCTGGTGAAACCTTCTGAGACCCTGAGCCTGA |
| | | CGTGCACCGTGAGCGGCGGCAGCATCACCAGCA |
| | | GCAACTACTACTGGGGCTGGATACGACAGCCCCC |
| | | TGGAAAGGGCCTAGAGTGGATCGGCAGCATCTAC |
| | | TACAGCGGCAACACCTACTACAACCCCAGCCTGA |
| | | AGAGCAGAGTCACCATCAGCGTGGACACCAGCA |
| | | AGAACCAGTTCAGCCTGAAGCTGAGCAGCGTGAC |
| | | CGCCGCCGACACAGCTGTTTACTATTGTGCCAGA |
| | | GAGTGGAGCCCCTACGAGAGCGAGGGCTTCGACT |
| | | ACTGGGGCCAGGGGACCCTGGTGACCGTTTCTTC |
| | | T |
| 20 | CD8 hinge | TTTPAPRPPTPAPTIASQPLSLRPEACRPAAGGAVHT |
| | | RGLDFACD |
| 21 | CD8 hinge coding | ACAACAACACCTGCTCCTCGGCCTCCTACACCAG |
| | sequence | CTCCTACAATTGCCAGCCAGCCACTGTCTCTGAG |
| | | GCCCGAAGCTTGCAGGCCTGCTGCTGGCGGAGCC |
| | | GTGCATACAAGAGGACTGGATTTCGCCTGCGAC |
| 22 | CD8 | IYIWAPLAGTCGVLLLSLVITLYC |
| | transmembrane | |
| | domain | |
| 23 | CD8 | ATCTACATCTGGGCACCTCTGGCTGGAACCTGTG |
| | transmembrane | GCGTGCTGCTGCTGAGCCTGGTCATCACCCTGTA |
| | domain coding | TTGC |
| | sequence | |
| 24 | CD28 | FWVLVVVGGVLACYSLLVTVAFIIFWV |
| | transmembrane | |
| | domain | |
| 25 | CD28 | TTTTGGGTGCTGGTGGTGGTTGGTGGAGTCCTGG |
| | transmembrane | CTTGCTATAGCTTGCTAGTAACAGTGGCCTTTATT |
| | domain coding | ATTTTCTGGGT G |
| | sequence | |
| 26 | 4-1BB | IISFFLALTSTALLFLLFFLTLRFSVV |
| | transmembrane | |
| | domain | |
| 27 | 4-1BB | ATCATCTCCTTCTTTCTTGCGCTGACcTCGACTGC |
| | transmembrane | GTTGCTCTTCCTGCTGTTCTTCCTCACGCTCCGTTT |
| | domain coding | CTCTGTTGTT |
| | sequence | |
| 28 | TNFR2 | FALPVGLIVGVTALGLLIIGVVNCVIMTQV |
| | transmembrane | |
| | domain | |
| 29 | TNFR2 | TTCGCTCTTCCAGTTGGACTGATTGTGGGTGTGAC |
| | transmembrane | AGCCTTGGGTCTACTAATAATAGGAGTGGTGAAC |
| | domain coding | T GTGTCATCAT GACCCAGGTG |
| | sequence | |
| 30 | CD3 zeta primary | RVKFSRSADAPAYQQGQNQLYNELNLGRREEYDV |
| | signaling domain | LDKRRGRDPEMGGKPRRKNPQEGLYNELQKDKMA |
| | | EAYSEIGMKGERRRGKGHDGLYQGLSTATKDTYD |
| | | ALHMQALPPR |
| 31 | CD3 zeta primary | AGAGTGAAGTTCAGCAGATCCGCCGACGCTCCTG |
| | signaling domain | CCTATCAGCAGGGCCAAAACCAGCTCTACAACGA |
| | coding sequence | GCTGAACCTGGGGAGAAGAGAAGAGTACGACGT |
| | | GCTGGACAAGCGGAGAGGCAGAGATCCTGAAAT |
| | | GGGCGGCAAGCCCAGACGGAAGAATCCTCAAGA |
| | | GGGCCTGTATAATGAGCTACAGAAAGACAAGAT |
| | | GGCAGAGGCCTACAGCGAGATCGGAATGAAGGG |
| | | CGAGCGCAGAAGAGGCAAGGGACACGATGGACT |
| | | GTACCAGGGCCTGAGCACCGCCACCAAGGATACC |
| | | TATGATGCCCTGCACATGCAGGCCCTGCCTCCAA |
| | | GA |
| 32 | CD28 | RSKRSRLLHSDYMNMTPRRPGPTRKHYQPYAPPRD |
| | costimulatory | FAAYRS |
| | signaling domain | |
| 33 | CD28 | CGGAGCAAGAGAAGCAGACTGCTGCACAGCGAC |
| | costimulatory | TACATGAACATGACCCCTAGACGGCCCGGACCTA |
| | signaling domain | CCAGAAAGCACTACCAGCCTTACGCTCCTCCTAG |
| | coding sequence | AGACTTCGCCGCCTACAGATCC |
| | (used in exemplary | |
| | codon optimized | |
| | variant V1 and V3) | |
| 34 | 4-1BB | KRGRKKLLYIFKQPFMRPVQTTQEEDGCSCRFPEEE |
| | costimulatory | EGGCEL |
| | signaling domain | |
| 35 | 4-1BB | AAACGGGGCAGAAAGAAACTCCTGTATATATTCA |
| | costimulatory | AACAACCATTTATGAGACCAGTACAAACTACTCA |
| | signaling domain | AGAGGAAGATGGCTGTAGCTGCCGATTTCCAGAA |
| | coding sequence | GAAGAAGAAGGAGGATGTGAACTG |
| 36 | CD27 | QRRKYRSNKGESPVEPAEPCRYSCPREEEGSTIPIQE |
| | costimulatory | DYRKPEPACSP |
| | signaling domain | |
| 37 | CD27 | CAACGAAGGAAATATAGATCAAACAAAGGAGAA |
| | costimulatory | AGTCCTGTGGAGCCTGCAGAGCCTTGTCGTTACA |
| | signaling domain | GCTGCCCCAGGGAGGAGGAGGGCAGCACCATCC |
| | coding sequence | CCATCCAGGAGGATTACCGAAAACCGGAGCCTGC |
| | | CTGCTCCCCC |
| 38 | TNFR2 | KKKPLCLQREAKVPHLPADKARGTQGPEQQHLLIT |
| | costimulatory | APSSSSSSLESSASALDRRAPTRNQPQAPGVEASGA |
| | signaling domain | GEARASTGSSDSSPGGHGTQVNVTCIVNVCSSSDHS |
| | | SQCSSQASSTMGDTDSSPSESPKDEQVPFSKEECAF |
| | | RSQLETPETLLGSTEEKPLPLGVPDAGMKPS |
| 39 | TNFR2 | AAAAAGAAGCCCTTGTGCCTGCAGAGAGAAGCC |
| | costimulatory | AAGGTGCCTCACTTGCCTGCAGATAAGGCCAGGG |
| | signaling domain | GTACACAGGGTCCCGAGCAGCAGCACCTCCTGAT |
| | coding sequence | CACAGCGCCGAGCTCCAGCAGCAGCTCCCTGGAG |
| | | AGCTCGGCCAGTGCGCTAGACAGAAGGGCCCCC |
| | | ACTCGGAACCAGCCACAGGCACCAGGCGTGGAG |
| | | GCGAGTGGGGCTGGGGAGGCGAGGGCAAGCACC |
| | | GGGAGCTCAGATTCTTCCCCTGGTGGTCATGGCA |
| | | CCCAGGTCAATGTCACCTGTATCGTGAACGTCTG |
| | | TAGCAGCTCTGACCACAGCTCACAGTGCTCCTCC |
| | | CAAGCCTCGTCCACAATGGGAGACACAGATTCCA |
| | | GCCCCTCGGAGTCCCCGAAGGACGAGCAAGTACC |
| | | CTTCTCCAAGGAGGAATGTGCCTTTCGGTCACAG |
| | | CTGGAGACGCCAGAGACCCTGCTGGGGAGCACC |
| | | GAAGAGAAGCCCCTGCCCCTTGGAGTGCCTGATG |
| | | CTGGGATGAAGCCCAGT |
| 40 | CD8 leader | MALPVTALLLPLALLLHAARP |
| | sequence | |
| 41 | CD8 leader | ATGGCTCTGCCTGTGACAGCTCTGCTGCTGCCTCT |
| | sequence | GGCTCTGCTTCTTCATGCCGCCAGACCA |
| 42 | HA tag | SYPYDVPDYAS |
| 43 | HA tag coding | TCTTACCCCTACGATGTGCCTGATTATGCATCT |
| | sequence | |
| 44 | CAR | QSVLTQPPSVSGAPGQRVTISCTGSSSNIGAGYDVH |
| | | WYQQLPGTAPKLLIYGNNNRPSGVPDRFSGSKSGTS |
| | | ASLAITGLQAEDEADYYCQSYDTGLSAWVFGGGTK |
| | | LTVLGGGGSGGGGSGGGGSQLQLQESGPGLVKPSE |
| | | TLSLTCTVSGGSITSSNYYWGWIRQPPGKGLEWIGSI |
| | | YYSGNTYYNPSLKSRVTISVDTSKNQFSLKLSSVTA |
| | | ADTAVYYCAREWSPYESEGFDYWGQGTLVTVSST |
| | | TTPAPRPPTPAPTIASQPLSLRPEACRPAAGGAVHTR |
| | | GLDFACDIYIWAPLAGTCGVLLLSLVITLYCRSKRS |
| | | RLLHSDYMNMTPRRPGPTRKHYQPYAPPRDFAAYR |
| | | SRVKFSRSADAPAYQQGQNQLYNELNLGRREEYD |
| | | VLDKRRGRDPEMGGKPRRKNPQEGLYNELQKDKM |
| | | AEAYSEIGMKGERRRGKGHDGLYQGLSTATKDTY |
| | | DALHMQALPPR |
| 45 | CAR coding | CAGAGCGTGCTGACACAGCCTCCATCTGTTTCTG |
| | sequence | GCGCCCCTGGCCAGAGAGTGACCATCTCTTGTAC |
| | | TGGCAGCAGCTCCAACATTGGAGCCGGCTATGAC |
| | | GTGCACTGGTACCAgCAGCTGCCTGGCACAGCCC |
| | | CTAAACTGCTGATCTACGGCAACAACAACAGACC |
| | | CAGCGGCGTGCCCGACAGATTTTCTGGCTCCAAG |
| | | AGCGGCACAAGCGCCAGCCTGGCTATTACTGGAC |
| | | TGCAGGCCGAGGACGAGGCCGACTACTACTGTCA |
| | | GAGCTACGACACCGGCCTGAGCGCCTGGGTTTTT |
| | | GGTGGAGGCACAAAACTGACAGTGCTGGGAGGC |
| | | GGAGGTTCTGGCGGCGGAGGAAGTGGTGGCGGA |
| | | GGCTCACAGCTGCAGCTACAGGAGAGCGGCCCC |
| | | GGCCTGGTGAAACCTTCTGAGACCCTGAGCCTGA |
| | | CGTGCACCGTGAGCGGCGGCAGCATCACCAGCA |
| | | GCAACTACTACTGGGGCTGGATACGACAGCCCCC |
| | | TGGAAAGGGCCTAGAGTGGATCGGCAGCATCTAC |
| | | TACAGCGGCAACACCTACTACAACCCCAGCCTGA |
| | | AGAGCAGAGTCACCATCAGCGTGGACACCAGCA |
| | | AGAACCAGTTCAGCCTGAAGCTGAGCAGCGTGAC |
| | | CGCCGCCGACACAGCTGTTTACTATTGTGCCAGA |
| | | GAGTGGAGCCCCTACGAGAGCGAGGGCTTCGACT |
| | | ACTGGGGCCAGGGGACCCTGGTGACCGTTTCTTC |
| | | TACAACAACACCTGCTCCTCGGCCTCCTACACCA |
| | | GCTCCTACAATTGCCAGCCAGCCACTGTCTCTGA |
| | | GGCCCGAAGCTTGCAGGCCTGCTGCTGGCGGAGC |
| | | CGTGCATACAAGAGGACTGGATTTCGCCTGCGAC |
| | | ATCTACATCTGGGCACCTCTGGCTGGAACCTGTG |
| | | GCGTGCTGCTGCTGAGCCTGGTCATCACCCTGTA |
| | | TTGCCGGAGCAAGAGAAGCAGACTGCTGCACAG |
| | | CGACTACATGAACATGACCCCTAGACGGCCCGGA |
| | | CCTACCAGAAAGCACTACCAGCCTTACGCTCCTC |
| | | CTAGAGACTTCGCCGCCTACAGATCCAGAGTGAA |
| | | GTTCAGCAGATCCGCCGACGCTCCTGCCTATCAG |
| | | CAGGGCCAAAACCAGCTCTACAACGAGCTGAAC |
| | | CTGGGGAGAAGAGAAGAGTACGACGTGCTGGAC |
| | | AAGCGGAGAGGCAGAGATCCTGAAATGGGCGGC |
| | | AAGCCCAGACGGAAGAATCCTCAAGAGGGCCTG |
| | | TATAATGAGCTACAGAAAGACAAGATGGCAGAG |
| | | GCCTACAGCGAGATCGGAATGAAGGGCGAGCGC |
| | | AGAAGAGGCAAGGGACACGATGGACTGTACCAG |
| | | GGCCTGAGCACCGCCACCAAGGATACCTATGATG |
| | | CCCTGCACATGCAGGCCCTGCCTCCAAGA |
| 46 | mCD8 hinge | TTTKPVLRTPSPVHPTGTSQPQRPEDCRPRGSVKGT |
| | | GLDFACD |
| 47 | mCD8 hinge | ACTACTACCAAGCCAGTGCTGCGAACTCCCTCAC |
| | coding sequence | CTGTGCACCCTACCGGGACATCTCAGCCCCAGAG |
| | | ACCAGAAGATTGTCGGCCCCGTGGCTCAGTGAAG |
| | | GGGACCGGATTGGACTTCGCCTGTGAT |
| 48 | mCD28 hinge | IEFMYPPPYLDNERSNGTIIHIKEKHLCHTQSSPKL |
| 49 | mCD28 hinge | ATCGAGTTCATGTACCCTCCACCTTACCTGGACA |
| | coding sequence | ACGAGAGAAGCAACGGCACCATCATCCACATCA |
| | | AAGAGAAGCACCTCTGTCACACCCAGAGCAGCCC |
| | | CAAGCTG |
| 50 | mCD8 | IYIWAPLAGICVALLLSLIITLICYT |
| | transmembrane | |
| | domain | |
| 51 | mCD8 | ATTTACATCTGGGCACCCTTGGCCGGAATCTGCG |
| | transmembrane | TGGCCCTTCTGCTGTCCTTGATCATCACTCTCATC |
| | domain coding | TGCTACACA |
| | sequence | |
| 52 | mCD28 | FWALVVVAGVLFCYGLLVTVALCVIWT |
| | transmembrane | |
| | domain | |
| 53 | mCD28 | TTTTGGGCTCTTGTGGTGGTGGCTGGCGTGCTGTT |
| | transmembrane | TTGTTACGGCCTGCTGGTCACAGTGGCCCTGTGT |
| | domain coding | GTGATTTGGACC |
| | sequence | |
| 54 | mCD28 | NSRRNRGGQSDYMNMTPRRPGLTRKPYQPYAPAR |
| | costimulatory | DFAAYRP |
| | domain | |
| 55 | mCD28 | AACAGCAGAAGAAACAGAGGCGGACAGAGCGAC |
| | costimulatory | TACATGAACATGACCCCTAGACGGCCTGGCCTGA |
| | domain coding | CAAGAAAGCCCTACCAGCCTTACGCTCCCGCCAG |
| | sequence | AGATTTCGCCGCCTACAGACCT |
| 56 | mCD3 zeta | RAKFSRSAETAANLQDPNQLYNELNLGRREEYDVL |
| | primary signaling | EKKRARDPEMGGKQQRRRNPQEGVYNALQKDKM |
| | domain | AEAYSEIGTKGERRRGKGHDGLYQGLSTATKDTYD |
| | | ALHMQTLAPR |
| 57 | mCD3 zeta | AGAGCCAAGTTCAGCAGATCCGCCGAAACAGCC |
| | primary signaling | GCCAACtTGCAaGATCCTAACCAGCTGTACAACGA |
| | domain coding | GCTGAACCTGGGGAGAAGAGAAGAGTACGACGT |
| | sequence | GCTGGAAAAGAAGAGGGCCAGAGATCCAGAGAT |
| | | GGGCGGCAAGCAGCAGAGAAGAAGAAACCCTCA |
| | | AGAGGGCGTGTACAACGCTCTGCAaAAAGACAAG |
| | | ATGGCCGAGGCCTACAGCGAGATCGGAACTAAG |
| | | GGCGAACGCAGAAGAGGCAAGGGCCACGATGGA |
| | | CTGTACCAGGGCCTGAGCACAGCCACCAAGGAC |
| | | ACATACGATGCCCTGCACATGCAGACACTGGCCC |
| | | CTAGATAG |
| 58 | CD25 leader | MDSYLLMWGLLTFIMVPGCQA |
| | sequence | |
| 59 | CD25 leader | ATGGATTCATACCTGCTGATGTGGGGACTGCTCA |
| | sequence | CGTTCATCATGGTGCCTGGCTGCCAGGCA |
| 60 | Igk leader | METPAQLLFLLLLWLPDTTG |
| | sequence | |
| 61 | Igk leader | ATGGAAACCCCAGCGCAGCTTCTCTTCCTCCTGCT |
| | sequence | ACTCTGGCTCCCAGATACCACCGGA |
| 62 | WPRE | Cgtgaggctccggtgcccgtcagtgggcagagcgcacategcccacagtccc |
| | | cgagaagttggggggaggggtcggcaattgaaccggtgcctagagaaggtgg |
| | | cgcggggtaaactgggaaagtgatgtcgtgtactggctccgcctttttcccgagg |
| | | gtgggggagaaccgtatataagtgcagtagtcgccgtgaacgttctttttcgcaac |
| | | gggtttgccgccagaacacaggtaagtgccgtgtgtggttcccgcgggcctggc |
| | | ctctttacgggttatggcccttgcgtgccttgaattacttccacgcccctggctgca |
| | | gtacgtgattcttgatcccgagcttcgggttggaagtggggggagagttcgagg |
| | | ccttgcgcttaaggagccccttcgcctcgtgcttgagttgaggcctggcctgggc |
| | | gctggggccgccgcgtgcgaatctggtggcaccttcgcgcctgtctcgctgcttt |
| | | cgataagtctctagccatttaaaatttttgatgacctgctgcgacgctttttttctg |
| | | gcaagatagtcttgtaaatgcgggccaagatctgcacactggtatttcggtttttgg |
| | | ggccgcgggcggcgacggggcccgtgcgtcccagcgcacatgttcggcgaggcg |
| | | gggcctgcgagcgcggccaccgagaatcggacgggggtagtctcaagctggc |
| | | cggcctgctctggtgcctggcctcgcgccgccgtgtatcgccccgccctgggcg |
| | | gcaaggctggcccggtcggcaccagttgcgtgagcggaaagatggccgcttcc |
| | | cggccctgctgcagggagctcaaaatggaggacgcggcgctcgggagagcg |
| | | ggcgggtgagtcacccacacaaaggaaaagggcctttccgtcctcagccgtcg |
| | | cttcatgtgactccacggagtaccgggcgccgtccaggcacctcgattagttctc |
| | | gagcttttggagtacgtcgtctttaggttggggggaggggttttatgcgatggagtt |
| | | tccccacactgagtgggtggagactgaagttaggccagcttggcacttgatgtaat |
| | | tctccttggaatttgccctttttgagtttggatcttggttcattctcaagcctcaga |
| | | cagtggttcaaagtttttttcttccatttcaggtgtcgtga |
| 63 | PGK-promoter | tccacggggttggggttgcgccttttccaaggcagccctgggtttgcgcagggac |
| | | gcggctgctctgggcgtggttccgggaaacgcagcggcgccgaccctgggtct |
| | | cgcacattcttcacgtccgttcgcagcgtcacccggatcttcgccgctacccttgtg |
| | | ggccccccggcgacgcttcctgctccgcccctaagtcgggaaggttccttgcgg |
| | | ttcgcggcgtgccggacgtgacaaacggaagccgcacgtctcactagtaccctc |
| | | gcagacggacagcgccagggagcaatggcagcgcgccgaccgcgatgggct |
| | | gtggccaatagcggctgctcagcagggcgcgccgagagcagcggccgggaa |
| | | ggggcggtgcgggaggcggggtgtggggcggtagtgtgggccctgttcctgc |
| | | ccgcgcggtgttccgcattctgcaagcctccggagcgcacgtcggcagtcggct |
| | | ccctcgttgaccgaatcaccgacctctctccccag |
| 64 | CAR-GFP- | MALPVTALLLPLALLLHAARPSYPYDVPDYASQSV |
| | Sequence (CAR#8- | LTQPPSVSGAPGQRVTISCTGSSSNIGAGYDVHWYQ |
| | CAR#11) (using | QLPGTAPKLLIYGNNNRPSGVPDRFSGSKSGTSASL |
| | scFV SEQ ID NO: | AITGLQAEDEADYYCQSYDTGLSAWVFGGGTKLT |
| | 15 and CAR SEQ | VLGGGGSGGGGSGGGGSQLQLQESGPGLVKPSETL |
| | ID NO: 44) | SLTCTVSGGSITSSNYYWGWIRQPPGKGLEWIGSIY |
| | | YSGNTYYNPSLKSRVTISVDTSKNQFSLKLSSVTAA |
| | | DTAVYYCAREWSPYESEGFDYWGQGTLVTVSSTTT |
| | | PAPRPPTPAPTIASQPLSLRPEACRPAAGGAVHTRGL |
| | | DFACDIYIWAPLAGTCGVLLLSLVITLYCRSKRSRLL |
| | | HSDYMNMTPRRPGPTRKHYQPYAPPRDFAAYRSR |
| | | VKFSRSADAPAYQQGQNQLYNELNLGRREEYDVL |
| | | DKRRGRDPEMGGKPRRKNPQEGLYNELQKDKMAE |
| | | AYSEIGMKGERRRGKGHDGLYQGLSTATKDTYDA |
| | | LHMQALPPRASGSGATNFSLLKQAGDVEENPGPMV |
| | | SKGEELFTGVVPILVELDGDVNGHKFSVSGEGEGD |
| | | ATYGKLTLKFICTTGKLPVPWPTLVTTLTYGVQCFS |
| | | RYPDHMKQHDFFKSAMPEGYVQERTIFFKDDGNY |
| | | KTRAEVKFEGDTLVNRIELKGIDFKEDGNILGHKLE |
| | | YNYNSHNVYIMADKQKNGIKVNFKIRHNIEDGSVQ |
| | | LADHYQQNTPIGDGPVLLPDNHYLSTQSALSKDPN |
| | | EKRDHMVLLEFVTAAGITLGMDELYK* |
| 65 | CAR-GFP- | ATGGCTCTGCCTGTGACAGCTCTGCTGCTGCCTCT |
| | Sequence (CAR#8- | GGCTCTGCTTCTTCATGCCGCCAGACCATCTTACC |
| | CAR#11) (using | CCTACGATGTGCCTGATTATGCATCTCAGAGCGT |
| | scFV SEQ ID NO: | GCTGACACAGCCTCCATCTGTTTCTGGCGCCCCTG |
| | 19 and CAR SEQ | GCCAGAGAGTGACCATCTCTTGTACTGGCAGCAG |
| | ID NO: 45) | CTCCAACATTGGAGCCGGCTATGACGTGCACTGG |
| | | TACCAgCAGCTGCCTGGCACAGCCCCTAAACTGC |
| | | TGATCTACGGCAACAACAACAGACCCAGCGGCGT |
| | | GCCCGACAGATTTTCTGGCTCCAAGAGCGGCACA |
| | | AGCGCCAGCCTGGCTATTACTGGACTGCAGGCCG |
| | | AGGACGAGGCCGACTACTACTGTCAGAGCTACGA |
| | | CACCGGCCTGAGCGCCTGGGTTTTTGGTGGAGGC |
| | | ACAAAACTGACAGTGCTGGGAGGCGGAGGTTCT |
| | | GGCGGCGGAGGAAGTGGTGGCGGAGGCTCACAG |
| | | CTGCAGCTACAGGAGAGCGGCCCCGGCCTGGTGA |
| | | AACCTTCTGAGACCCTGAGCCTGACGTGCACCGT |
| | | GAGCGGCGGCAGCATCACCAGCAGCAACTACTA |
| | | CTGGGGCTGGATACGACAGCCCCCTGGAAAGGG |
| | | CCTAGAGTGGATCGGCAGCATCTACTACAGCGGC |
| | | AACACCTACTACAACCCCAGCCTGAAGAGCAGA |
| | | GTCACCATCAGCGTGGACACCAGCAAGAACCAGT |
| | | TCAGCCTGAAGCTGAGCAGCGTGACCGCCGCCGA |
| | | CACAGCTGTTTACTATTGTGCCAGAGAGTGGAGC |
| | | CCCTACGAGAGCGAGGGCTTCGACTACTGGGGCC |
| | | AGGGGACCCTGGTGACCGTTTCTTCTACAACAAC |
| | | ACCTGCTCCTCGGCCTCCTACACCAGCTCCTACA |
| | | ATTGCCAGCCAGCCACTGTCTCTGAGGCCCGAAG |
| | | CTTGCAGGCCTGCTGCTGGCGGAGCCGTGCATAC |
| | | AAGAGGACTGGATTTCGCCTGCGACATCTACATC |
| | | TGGGCACCTCTGGCTGGAACCTGTGGCGTGCTGC |
| | | TGCTGAGCCTGGTCATCACCCTGTATTGCCGGAG |
| | | CAAGAGAAGCAGACTGCTGCACAGCGACTACAT |
| | | GAACATGACCCCTAGACGGCCCGGACCTACCAGA |
| | | AAGCACTACCAGCCTTACGCTCCTCCTAGAGACT |
| | | TCGCCGCCTACAGATCCAGAGTGAAGTTCAGCAG |
| | | ATCCGCCGACGCTCCTGCCTATCAGCAGGGCCAA |
| | | AACCAGCTCTACAACGAGCTGAACCTGGGGAGA |
| | | AGAGAAGAGTACGACGTGCTGGACAAGCGGAGA |
| | | GGCAGAGATCCTGAAATGGGCGGCAAGCCCAGA |
| | | CGGAAGAATCCTCAAGAGGGCCTGTATAATGAGC |
| | | TACAGAAAGACAAGATGGCAGAGGCCTACAGCG |
| | | AGATCGGAATGAAGGGCGAGCGCAGAAGAGGCA |
| | | AGGGACACGATGGACTGTACCAGGGCCTGAGCA |
| | | CCGCCACCAAGGATACCTATGATGCCCTGCACAT |
| | | GCAGGCCCTGCCTCCAAGAGCTAGcggaagcggagctac |
| | | taacttcagcctgctgaagcaggctggagacgtggaggagaaccctggacctat |
| | | ggtgagcaagggcgaggagctgttcaccggggtggtgcccatcctggtcgagc |
| | | tggacggcgacgtaaacggccacaagttcagcgtgtccggcgagggcgaggg |
| | | cgatgccacctacggcaagctgaccctgaagttcatctgcaccaccggcaagct |
| | | gcccgtgccctggcccaccctcgtgaccaccctgacctacggcgtgcagtgctt |
| | | cagccgctaccccgaccacatgaagcagcacgacttcttcaagtccgccatgcc |
| | | cgaaggctacgtccaggagcgcaccatcttcttcaaggacgacggcaactacaa |
| | | gacccgcgccgaggtgaagttcgagggcgacaccctggtgaaccgcatcgag |
| | | ctgaagggcatcgacttcaaggaggacggcaacatcctggggcacaagctgga |
| | | gtacaactacaacagccacaacgtctatatcatggccgacaagcagaagaacgg |
| | | catcaaggtgaacttcaagatccgccacaacatcgaggacggcagcgtgcagct |
| | | cgccgaccactaccagcagaacacccccateggcgacggccccgtgctgctgc |
| | | ccgacaaccactacctgagcacccagtccgccctgagcaaagaccccaacgag |
| | | aagcgcgatcacatggtcctgctggagttcgtgaccgccgccgggatcactctc |
| | | ggcatggacgagctgtacaagtaa |
| 66 | scFv coding | CAATCTGTCCTGACCCAGCCACCCTCTGTTAGTG |
| | sequence | GAGCACCTGGACAGAGAGTGACCATCAGTTGTAC |
| | | TGGGAGCAGCTCCAACATTGGAGCCGGCTATGAC |
| | | GTCCACTGGTACCAGCAGCTTCCAGGCACTGCTC |
| | | CCAAACTCCTGATCTACGGCAACAACAATCGACC |
| | | AAGTGGGGTCCCAGACAGGTTCAGTGGTTCCAAA |
| | | TCTGGGACATCAGCATCTCTGGCCATTACAGGCC |
| | | TCCAGGCTGAAGATGAAGCAGACTACTACTGCCA |
| | | GTCATATGACACAGGGCTCTCTGCATGGGTGTTT |
| | | GGTGGAGGGACAAAGCTGACTGTGCTGGGAGGC |
| | | GGAGGTTCTGGCGGCGGAGGAAGTGGTGGCGGA |
| | | GGCTCACAGCTGCAGTTGCAGGAGTCTGGGCCTG |
| | | GACTTGTCAAACCCTCTGAAACACTGTCACTGAC |
| | | TTGCACAGTCTCTGGAGGAAGCATCACCTCTTCA |
| | | AACTACTACTGGGGCTGGATCAGACAGCCACCAG |
| | | GGAAGGGCCTGGAGTGGATTGGAAGCATCTACTA |
| | | CAGCGGGAACACTTACTACAATCCCTCTCTGAAA |
| | | TCAAGGGTCACCATCAGTGTGGACACCTCCAAGA |
| | | ACCAGTTCTCACTCAAGCTGAGCTCAGTGACAGC |
| | | TGCAGACACTGCTGTTTATTATTGTGCAAGAGAG |
| | | TGGAGCCCCTACGAGTCTGAAGGCTTTGACTACT |
| | | GGGGCCAAGGCACTCTTGTGACCGTGTCCAGC |
| 67 | CD8 hinge coding | ACCACTACTCCTGCTCCTCGGCCTCCTACACCAGC |
| | sequence | TCCTACTATAGCCAGTCAGCCACTGAGCCTGAGG |
| | | CCTGAGGCCTGTAGACCAGCAGCTGGCGGGGCTG |
| | | TTCATACAAGAGGCCTGGATTTTGCCTGTGAC |
| 68 | CD8 | ATCTACATATGGGCACCTCTTGCTGGTACATGTG |
| | transmembrane | GAGTGCTGCTGCTGAGCTTGGTCATCACCCTCTA |
| | domain coding | CTGC |
| | sequence | |
| 69 | CD3 zeta primary | AGAGTGAAGTTCTCAAGAAGTGCCGACGCCCCAG |
| | signaling domain | CCTATCAGCAAGGCCAGAACCAGCTGTATAATGA |
| | coding sequence | ATTGAACCTGGGAAGGAGGGAGGAATATGATGT |
| | | TCTGGACAAGAGGAGAGGCAGGGACCCTGAAAT |
| | | GGGCGGGAAACCACGAAGAAAGAACCCCCAGGA |
| | | AGGACTTTACAATGAGCTCCAGAAAGACAAGAT |
| | | GGCTGAAGCCTATTCTGAGATCGGCATGAAGGGA |
| | | GAGCGCAGAAGAGGCAAAGGGCACGACGGCCTG |
| | | TATCAAGGGCTCTCCACAGCTACCAAAGACACCT |
| | | ATGATGCTCTGCACATGCAGGCTCTGCCTCCCCG |
| | | C |
| 70 | CD28 | CGGAGCAAAAGAAGCAGACTCCTCCATTCTGACT |
| | costimulatory | ACATGAATATGACACCCAGGAGACCAGGACCAA |
| | signaling domain | CAAGGAAGCACTACCAACCTTATGCTCCTCCTCG |
| | coding sequence | GGATTTTGCCGCCTATAGATCC |
| 71 | scFv coding | CAGAGTGTGCTCACACAACCTCCATCTGTTTCTG |
| | sequence | GTGCTCCTGGGCAGAGAGTCACCATCTCTTGTAC |
| | | TGGGTCGTCCTCCAATATTGGAGCTGGCTATGAT |
| | | GTCCACTGGTATCAACAGCTGCCTGGGACAGCCC |
| | | CTAAACTCCTCATCTATGGCAACAATAACAGGCC |
| | | CAGTGGTGTGCCTGACAGATTTTCTGGGTCCAAG |
| | | TCAGGCACAAGTGCCTCATTGGCTATTACTGGAC |
| | | TGCAGGCTGAGGATGAGGCAGACTACTACTGTCA |
| | | GTCATATGACACTGGCCTAAGTGCCTGGGTTTTT |
| | | GGTGGAGGCACAAAACTGACAGTCCTGGGAGGT |
| | | GGAGGTTCTGGCGGTGGAGGGAGTGGTGGCGGA |
| | | GGCTCACAACTTCAGCTACAAGAGAGTGGCCCTG |
| | | GGCTTGTGAAACCTTCTGAGACCCTCTCTCTGAC |
| | | GTGCACAGTGTCTGGAGGAAGCATCACCAGTAGT |
| | | AACTACTACTGGGGCTGGATACGACAGCCCCCTG |
| | | GAAAGGGGCTAGAGTGGATTGGCAGCATCTATTA |
| | | TTCAGGCAACACGTATTATAATCCCAGCCTCAAG |
| | | AGCAGAGTCACCATCTCTGTGGACACGTCAAAGA |
| | | ACCAGTTCTCTCTAAAGCTGTCCAGTGTGACTGCT |
| | | GCAGACACAGCTGTTTACTATTGTGCCAGAGAAT |
| | | GGTCTCCTTATGAGAGTGAGGGCTTTGACTACTG |
| | | GGGACAGGGAACCCTGGTGACAGTCTCTTCT |
| 72 | (GGS)2-Linker | GGSGGS |

1. An isolated anti-IL-23 receptor (IL-23R) antibody or antigen-binding fragment thereof, wherein
the heavy chain variable region (VH) of the antibody or fragment comprises complementary-determining regions (HCDRs) 1-3 comprising SEQ ID NOs: 1-3, respectively; or any HCDR having an amino acid sequence that shares at least about 90% of identity with one of SEQ ID NOs: 1-3; and
the light chain variable region (VL) of the antibody or fragment comprises complementary-determining regions (LCDRs) 1-3 comprising SEQ ID NOs: 4-6, respectively; or any LCDR having an amino acid sequence that shares at least about 90% of identity with one of SEQ ID NOs: 4-6.

2. An isolated anti-IL-23 receptor (IL-23R) antibody or antigen-binding fragment thereof, wherein
the heavy chain variable region (VH) of the antibody or fragment comprises complementary-determining regions (HCDRs) 1-3 comprising SEQ ID NOs: 1-3, respectively; and the light chain variable region (VL) of the antibody or fragment comprises complementary-determining regions (LCDRs) 1-3 comprising SEQ ID NOs: 4-6, respectively.

3. The isolated anti-IL-23R antibody or antigen-binding fragment thereof according to claim 1, wherein said antibody or antigen-binding fragment is capable of binding mouse and human IL-23R.

4. The isolated anti-IL-23R antibody or antigen-binding fragment thereof according to claim 1, wherein said antibody or antigen-binding fragment is capable of binding to a human IL-23R alpha subunit with an EC50 of less than 40 nM.

5. The isolated anti-IL-23R antibody or antigen-binding fragment thereof according to claim 1, wherein said antibody or antigen-binding fragment is capable of binding to a mouse IL-23R alpha subunit with an EC50 of less than 60 nM.

6. The isolated anti-IL-23R antibody or antigen-binding fragment thereof according to claim 1, wherein
said VH comprises SEQ ID NO: 7 or an amino acid sequence at least about 90% identical thereto, and
said VL comprises SEQ ID NO: 8 or any amino acid sequence at least about 90% of identical thereto.

7. The isolated anti-IL-23R antibody or antigen-binding fragment thereof according to claim 6, wherein
said VH comprises SEQ ID NO: 7, and
said VL comprises SEQ ID NO: 8.

8. The isolated anti-IL-23R antibody or antigen-binding fragment thereof according to claim 1, wherein said antibody or antigen-binding fragment is an scFv comprising SEQ ID NO: 15 or any amino acid sequence at least about 95% identical thereto.

9. The isolated anti-IL-23R antibody or antigen-binding fragment thereof according to claim 8, wherein said antibody or antigen-binding fragment is an scFv comprising SEQ ID NO: 15.

10. A chimeric antigen receptor (CAR) comprising:
(i) an extracellular domain comprising an anti-IL-23R antibody or antigen-binding fragment thereof according to claim 1;
(ii) a transmembrane domain; and
(iii) a cytoplasmic domain comprising an intracellular signaling domain.

11. The CAR according to claim 10, further comprising a leader sequence.

12. The CAR according to claim 10, wherein the extracellular domain comprises an scFv comprising SEQ ID NO: 15.

13. The CAR according to claim 10, wherein the intracellular signaling domain comprises
a human CD28 costimulatory signaling domain, optionally comprising SEQ ID NO: 32 or an amino acid sequence at least about 90% identical thereto, and/or
a human CD3 zeta domain, optionally comprising SEQ ID NO: 30 or an amino acid sequence at least about 90% identical thereto.

14. The CAR according to claim 10, wherein the transmembrane domain is derived from human CD8, optionally comprising SEQ ID NO: 22 or an amino acid sequence at least about 90% identical thereto.

15. The CAR according to claim 10, wherein the leader sequence comprises an amino acid sequence derived from a CD8 leader sequence, optionally comprising SEQ ID NO: 40 or an amino acid sequence at least about 95% identical thereto.

16. The CAR according to claim 10, wherein the leader sequence comprises an amino acid sequence derived from a CD25 leader sequence, optionally comprising SEQ ID NO: 58 or an amino acid sequence at least about 95% identical thereto.

17. A chimeric antigen receptor (CAR) comprising:
(i) an anti-IL-23R scFv, optionally comprising SEQ ID NO: 15,
(ii) a hinge domain derived from human CD8, optionally comprising SEQ ID NO: 20,
(iii) a transmembrane domain derived from human CD8, optionally comprising SEQ ID NO: 22,
(iv) an intracellular signaling domain comprising a human CD28 costimulatory signaling domain, optionally comprising SEQ ID NO: 32, and a human CD3 zeta domain, optionally comprising SEQ ID NO: 30, and
(v) optionally a tag and/or a leader sequence.

18. A chimeric antigen receptor (CAR) comprising:
(i) an anti-IL-23R scFv, optionally comprising SEQ ID NO: 15,
(ii) a hinge domain derived from human CD8, optionally comprising SEQ ID NO: 20,
(iii) a transmembrane domain derived from human CD8, optionally comprising SEQ ID NO: 22,
(iv) an intracellular signaling domain comprising a human CD28 costimulatory signaling domain, optionally comprising SEQ ID NO: 32, and a human CD3 zeta domain, optionally comprising SEQ ID NO: 30, and
(v) a leader sequence derived from CD8, optionally comprising SEQ ID NO: 40.

19. A chimeric antigen receptor (CAR) comprising:
(i) an anti-IL-23R scFv, optionally comprising SEQ ID NO: 15,
(ii) a hinge domain derived from human CD8, optionally comprising SEQ ID NO: 20,
(iii) a transmembrane domain derived from human CD8, optionally comprising SEQ ID NO: 22,
(iv) an intracellular signaling domain comprising a human CD28 costimulatory signaling domain, optionally comprising SEQ ID NO: 32, and a human CD3 zeta domain, optionally comprising SEQ ID NO: 30, and
(v) a leader sequence derived from CD25, optionally comprising SEQ ID NO: 58.

20. A nucleic acid molecule encoding the antibody or antigen-binding fragment according to claim 1.

21. A vector comprising the nucleic acid molecule according to claim 20.

22. An immune cell expressing the CAR according to claim 10.

23. A cell comprising the nucleic acid molecule according to claim 20 or a vector comprising the nucleic acid molecule.

24. The cell according to claim 23, wherein the cell is an immune cell.

25. A composition comprising the immune cell according to claim 22.

26. A composition comprising the cell according to claim 23.

27. A composition comprising the cell according to claim 23-24.

28. (canceled)

29. A method for treating a disorder or disease in a subject in need thereof, wherein the method comprises administering to said patient the immune cell according to claim 22.

30. The method according to claim 29, wherein the disease or disorder is mediated by IL-23R-expressing cells in the subject in need thereof, optionally wherein said disease or disorder is an autoimmune or inflammatory disease or disorder.

31. The method according to claim 29, wherein said disease or disorder is selected from the group consisting of inflammatory bowel diseases, lupus, arthritis, Sjogren's syndrome, systemic sclerosis, multiple sclerosis, ankylosing spondylitis, type 1 diabetes, autoimmune thyroid disorders, myasthenia gravis, psoriasis, psoriatic arthritis, skin diseases and uveitis, further optionally wherein the disease is Crohn's disease.
